(19) United States
(12) Patent Application Publication
(10) Pub. No.: US 2025/0135011 A1
(43) Pub. Date: May 1, 2025

(54) HYDROPHILIC TETRAZINE-FUNCTIONALIZED PAYLOADS FOR PREPARATION OF TARGETING CONJUGATES

(71) Applicants: EUROPEAN MOLECULAR BIOLOGY LABORATORY, Heidelberg (DE); VERAXA BIOTECH GMBH, Heidelberg (DE)

(72) Inventors: Edward A. LEMKE, Mainz (DE); Carsten SCHULTZ, Portland, OR (US); Christine KÖHLER, Forst (DE); Paul Felix SAUTER, Freiburg (DE)

(21) Appl. No.: 18/716,887

(22) PCT Filed: Dec. 8, 2022
(86) PCT No.: PCT/EP2022/084914

§ 371 Date: Jun. 5, 2024

(30) Foreign Application Priority Data
Dec. 8, 2021 (EP) 21213081.9

(51) Int. Cl. A61K 47/54; A61K 47/68

(52) U.S. Cl. CPC A61K 47/545; A61K 47/68031; A61K 47/68033; A61K 47/6855; A61K 47/6889

(57) ABSTRACT

The invention relates to the field of bioconjugation of functional entities (payloads) to targeting agents, in particular biological targeting agents, such as antibody drug conjugates (ADCs), where one or more payload molecules are conjugated to a targeting agent, as for example a monoclonal antibody. More particularly, the present invention relates to novel hydrophilic tetrazine molecules and their preparation, which tetrazines allow a more efficient conjugation of payload molecules to targeting agents, like monoclonal antibodies. The present invention also relates to particular tetrazine intermediates useful for the preparation of correspondingly functionalized payload molecules. The present invention also relates to respective conjugates, in particular bio-conjugates and methods of their preparation. The invention also relates to the use of such conjugates of the present invention for use in medicine, to corresponding pharmaceutical compositions as well as to corresponding diagnostic and analytical kits.

### FIELD OF THE INVENTION

The invention relates to the field of bioconjugation of functional entities (payloads) to targeting agents, in particular biological targeting agents, such as antibody drug conjugates (ADCs), where one or more payload molecules are conjugated to a targeting agent, as for example monoclonal antibody. More particularly, the present invention relates to novel hydrophilic tetrazine molecules and their preparation, which tetrazines allow a more efficient conjugation of payload molecules to targeting agents, like monoclonal antibodies. The present invention also relates to particular tetrazine intermediates useful for the preparation of correspondingly functionalized payload molecules. The present invention also relates to respective conjugates, in particular bio-conjugates and methods of their preparation. The invention also relates to the use of such conjugates of the present invention for use in medicine, to corresponding pharmaceutical compositions as well as to corresponding diagnostic and analytical kits.

### BACKGROUND OF THE INVENTION

ADCs are a fast growing class of oncology therapeutics that perceive major attention, which is reflected in the growing number of approved ADC drugs and increasing numbers of clinical trials.

The required conjugation of the respective payload to monoclonal antibodies (mAbs) is often done via random attachment. This typically leads to inhomogeneity regarding the present species of ADCs and drug-to-antibody ratios (DARs) vary from completely unmodified mAbs to unfavorable high numbers of cytotoxic molecules attached, which leads to problems with batch-to-batch variability and can cause aggregation and concomitant side effects like fast clearance, immunogenicity or hepatoxicity.

A solution to overcome these limitations is the production of homogenous ADCs/radioimmunoconjugates (RICs) via site-specific conjugation methods. Site-specific ligation methods offer the possibility for tight control of the DAR, pharmacokinetic properties and production of uniform batches of administered drugs. This also leads to an improvement of the therapeutic index, because side effects of unwanted species of ADCs are erased.

Another substantial component of every ADC is the linker used for attachment of the payload.

Generally, there is differentiation between non-cleavable linkers, releasing the cargo only after proteasomal degradation, and cleavable linkers, releasing the parent drug via enzymatic, reductive or acidic cleavage. Moreover, linker chemistry also influences the properties of the actual released active metabolite. Increasing hydrophilicity for example leads to decreased rates diffusion across membranes and higher retention inside the cell, as well as less sensitivity to multi drug resistance mechanism of cancer cells.

The inventors already reported on genetically encoding of a strained cyclooctyne-lysine derivative for click reactions into proteins (T. Plass, S. Milles, C. Koehler, C. Schultz, E. A. Lemke, Angew. Chem. Int. Ed. 2011, 50, 3878-81) and the synthesis and genetically encoding of ncAAs that can undergo (strain-promoted) inverse-electron-demand Diels-Alder cycloadditions (IEDDA) with 1,2,4,5-tetrazines (T. Plass, S. Milles, C. Koehler, J. Szymański, R. Mueller, et al., Angew. Chem. Int. Ed. 2012, 51, 4166-70; I. Nikić, T. Plass, O. Schraidt, J. Szymański, J. A. G. Briggs, et al., Angew. Chem. Int. Ed. 2014, 53, 2245-9; E. Kozma, I. Nikić, B. R. Varga, I. V. Aramburu, J. H. Kang, et al., ChemBioChem 2016, 17, 1518-24; J.-E. Hoffmann, T. Plass, I. Nikić, I. V. Aramburu, C. Koehler, et al., Chem. Eur. J. 2015, 21, 12266-70). Extension of this GCE technology led to the site-specific introduction of such ncAAs into unglycosylated immunoglobulins produced by insect cells and subsequent modification via this click chemistry (C. Koehler, P. F. Sauter, M. Wawryszyn, G. E. Girona, K. Gupta, et al., Nat. Methods 2016, 13, 997-1000.).

Mao et al. describe in Angew. Chem Int Ed (2019), 58, 1106 the organocatalytic and scalable syntheses of unsymmetrical, 1,2,4,5-tetrazines by thiol-containing promoters. In particular, a series of unsymmetrically substituted 1,2,4,5-tetrazines derivatives has been synthesized by applying 3-mercapto propionic acid as catalyst for the reaction of 2 different nitrile educts with hydrazine hydrate in an ethanol solution and subsequent oxidation with sodium nitrite. One particular compound was a 1,2,4,5-tetrazines derivative, substituted in position 1 by a methyl phosphonate group and in position 6 by a methyl group. This phosphonate precursor was further derivatized via a Homer-Wadsworth-Emmons reaction to introduce side chains containing different trans-alkene moieties.

US2019247513A1 discloses tetrazine compounds and dienophile capable of undergoing inverse electron demand Diels Alder reaction with the said tetrazines and their use in bio-orthogonal drug activation. In particular, the Compounds 333 and 14.5 to 14.7 relate to tetrazine derivatives presenting a fluorescent payload, spaced apart from the tetrazine core by a spacer of various chemical nature, like aryl or peptidyl or alkyl-based spacers as well as polyoxyalkyl-based hydrophylic groups.

WO2020256544A1 relates to substituted tetrazines characterized by high click conjugation yield. The tartrazine core is attached to pyridyl-based spacers, themselves linked to a payload R87 (such as a cytotoxic drug) and/or polyoxyalkyl hydrophilic groups through glutaryl groups.

Mao et al. (Angew. Chem Int Ed (2021), 60 2393-2397) as well as WO2020239039A1 disclose tetrazine compounds in which an hydrophilic or chemical moieties capable of forming a chemical bond (such as carboxyl, hydroxyl or phosphate-based groups) are spaced apart from the tetrazine core by alkyl-based spacers. In that regard, Mao et al describes also compounds in which said hydrophilic or chemical moieties capable of forming a chemical bond are replaced by fluorescent groups.

Shainyan, B. A et al (CHEMICAL ABSTRACTS SERVICE, COLUMBUS, OHIO, US; Data base accession no. 1983: 453711) relates to tetrazines presenting alkylsulphone groups as para-substituents.

WO2014081301A1 discloses tetrazines whereby the tetrazine core is linked to various hydrophilic or chemical moieties capable of forming a chemical bond via aryl-based spacers.

Oller-Salvia et. al (Angew. Chem Int Ed (2018), 57 2831-2834) relates to the field of antibody drug conjugates and discloses a construct in which trastuzumab was site-selectively conjugated to tetrazine-modified monomethyl auristatin E (MMAE) via inverse-electron demand Diels-Alder cycloaddition. The tetrazine handle used for said site-specific conjugation consists of an aryl moiety attached to the tetrazine core.

Handula et al (Molecules 2021, 26, 4640) relates to the use of bio-orthogonal reactions such as tee IEDDA reactions in pre-targeting strategies. Various tetrazine surrogates are disclosed as suitable dienes, which dienes are characterized by the presence of a p-substituted tetrazine core presenting alkyl and/or aryl spacers. FIG. 2 of the disclosure relates to a classification of said tetrazines surrogates based on the respective reactivity.

There is a need for further improved 1,2,4,5-tetrazines derivatives which allow more favorable conjugation of 1,2,4,5-tetrazines functionalized payload molecules to correspondingly functionalized targeting molecules via bioorthogonal reaction. In particular, there is a need of further improved 1,2,4,5-tetrazines derivatives, which allow such bioorthogonal reaction to proceed via Strain-promoted Inverse Electron-Demand Diels-Alder cycloaddition (SPIEDAC) without greater steric hindrance in aqueous optionally buffered environment. More particularly, there is a need for such tetrazine derivatives, which allow, due to their increased hydrophilicity, the conjugation of more hydrophobic payloads, and, in the case of pharmacologically active conjugates provide for a better functional profile of such conjugate in vivo.

### SUMMARY OF THE INVENTION

The above-mentioned problem was surprisingly solved by the provision of 1,2,4,5-tetrazines functionalized payload molecules carrying as tetrazine C-substituent a small hydrophilic group, as for example a phosphonate residue. The invention enables easy use of payloads for bioorthogonal bioconjugation via SPIEDAC in aqueous buffers without the need for possibly disruptive added organic solvents and therefore use of sensitive biological agents. It also aids in prevention of aggregation and better solubility of formed bioconjugated agents. Due to its relatively small size it should not exhibit much steric hindrance compared to bigger solubilizing units, like PEGs, glycosides, etc. and allow for attachment of minimal size payloads. For example, no bulky PEG linkers within the conjugate are necessarily required, which in turn allows to reduce the size of the final payload or respective targeting conjugate. Similar sized payloads based on corresponding methyl tetrazine groups cannot be conjugated to the antibody under the same conditions. It was also observed that an increase of hydrophilicity in the claimed manner allows to provide conjugates, in particular ADCs, associated with improved pharmacokinetics, while at the same time the smaller size of the hydrophilic group of the tetrazine the entire payload is better masked by the antibody.

More particularly, the above-mentioned problem is solved by the provision of derivatives of phosphonate group carrying tetrazines further carrying as chemical moieties allowing for coupling with a payload molecule, for example via amide coupling or formation of carbamates. Corresponding tetrazine-functionalized payload molecules are represented by the general formula I as referred to herein below; the corresponding tetrazine-functionalized intermediates are represented by the general formula II as referred to herein below.

### BRIEF DESCRIPTION OF THE FIGURES

FIG. 1: IEDDA of a TCO derivative, incorporated site-specifically in a mAb, with a 1,2,4,5-tetrazine.

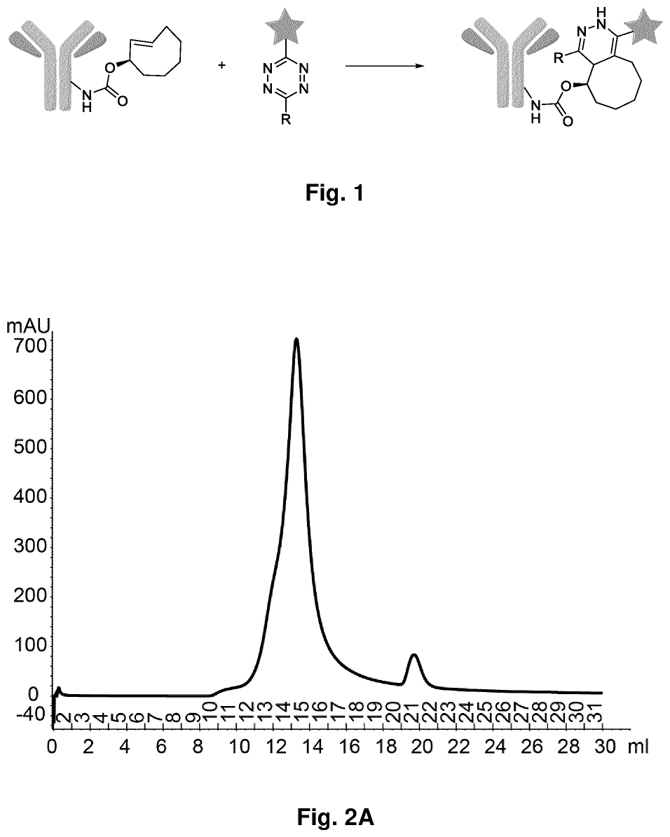

FIG. 2: Purification of TrastuzumabA132TCO*-5; A: Superdex S200 run, B: Coomassie stained SDS-PAGE analyzing fractions 10-20 of the S200 run

FIG. 3: Purification of TrastuzumabA132TCO*-11: Superdex S200 run, B: Coomassie stained SDS-PAGE analyzing fractions 10-20 of the S200 run

FIG. 4: Cell cytoxicity assay; Shown are the measurements for Trastuzumab-5 (=compound 5), Trastuzumab-11 (=compound 11) and Trastuzumab WT.

### DETAILED DESCRIPTION OF THE INVENTION

### A. Abbreviations

ADC=antibody drug conjugate
APC=antibody payload conjugate
aq.=aqueous
Bps=base pairs
BCN=2-amino-6-(9-biocyclo[6.1.0]non-4-ynylmethoxycarbonylamino)hexanoid acid
BOC=2-amino-6-(tert-butoxycarbonylamino)hexanoic acid, in the examples “BOC” specifically designates (2S)-2-amino-6-(tert-butoxycarbonylamino)hexanoic acid=Boc-L-Lys-
OH=N-α-tert-butyloxycarbonyl-L-lysine
conc.=concentrated
DAR=Drug-to-antibody ratio
DCM=dichloromethane
DDQ=2,3-Dichloro-5,6-dicyano-1,4-benzoquinone
DIPEA=N,N-diisopropylethylamine
DMF=dimethylformamide
DMSO=dimethylsulfoxide
EDC=1-Ethyl-3-(3-dimethylaminopropyl)carbodiimide
eq.=equivalent(s)
EtOH=ethanol
GCE=genetic code expansion
h=hour(s)
HATU=1-[Bis(dimethylamino)methylene]-1H-1,2,3-triazolo[4,5-b]pyridinium 3-oxide hexafluorophosphate, a coupling agent
HOBt=Hydroxybenzotriazole
IEDDA=Inverse Electron-Demand Diels-Alder Cycloaddition
kDa=kilo Dalton
min=minutes
MMAE=Monomethyl auristatin E ((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide), a anti-neoplastic agent
MeOH=methanol
ncAA=non-canonical amino acid
NES=nuclear export signal
NLS=nuclear localization signal
O-tRNA=orthogonal tRNA
O—RS=orthogonal RS
PBS=phosphate buffered saline
PMSF=phenylmethylsulfonylfluoride
PNP Chloroformate=4-Nitrophenyl chloroformate
POI=polypeptide of interest,
pRS=prokaryotic RS
ptRNA=prokaryotic tRNA
PyIRS=pyrrolysyl tRNA synthetase
PyIRS^{AF}=mutant M. mazei pyrrolysyl tRNA synthetase comprising amino acid substitutions Y306A and Y384F
RCF (rcf)=relative centrifugal force
RP-HPLC=reversed phase high-performance liquid chromatography
RS=aminoacyl tRNA synthetase
RT=room/ambient temperature (20-25° C.)
SCO=2-amino-6-(cyclooct-2-yn-1-yloxycarbonylamino)hexanoic acid

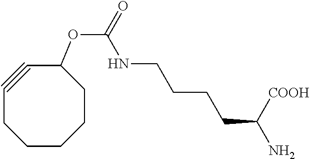 [SMILES: N[C@@H](CCCCNC(=O)OC1C#CCCCCC1)C(=O)O]

SDS-PAGE=sodium sodecyl sulfate polyacrylamide gel electrophoresis
SPIEDAC=Strain-promoted Inverse Electron-Demand Diels-Alder cycloaddition
5-TAMRA=5-Carboxytetramethylrhodamine
5-TAMRA-OSu=5-Carboxytetramethylrhodamine N-succinimidyl ester, a fluorophore
TCO=Trans-cyclooctene
TCO-Lys=N—ε-((trans-Cyclooct-4-en-1-yloxy)carbonyl)-L-lysine
TCO*-Lys=N—ε-((trans-Cyclooct-2-en-1-yloxy)carbonyl)-L-lysine
TCO^{#}-Lys=N—ε-((trans-Cyclooct-3-en-1-yloxy)carbonyl)-L-lysine
TCO-E-Lys=N6-((((R,E)-cyclooct-4-en-1-yl)oxy)carbonyl)-L-lysine

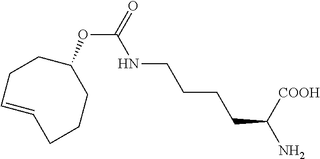

TCO*A-Lys=N6-((((S,E)-cyclooct-2-en-1-yl)oxy)carbonyl)-L-lysine

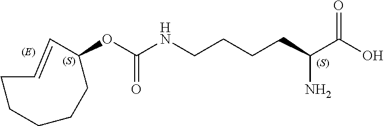

TFA=trifluoroacetic acid
THF=tetrahydrofurane
TLC=thin layer chromatography
tRNA^{PyI}=tRNA that can be acylated with pyrrolysine by a wild-type or modified PyIRS and has an anticodon that, for site-specific incorporation of the ncAA into a POI, is preferably the reverse complement of a selector codon.
UNAA=unnatural amino acid, synonym to ncAA
U6 promoter=promoter that normally controls expression of the U6 RNA (a small nuclear RNA) in mammalian cells
UHPLC-MS=Ultra High Performance Liquid Chromatography/Mass Spectrometry

### B. Definitions

### B.1 General Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The terms “purified”, “substantially purified,” and “isolated” as used herein refer to the state of being free of other, dissimilar compounds with which a compound of the invention is normally associated in its natural state, so that the “purified”, “substantially purified,” and “isolated” subject comprises at least 0.5%, 1%, 5%, 10%, or 20%, or at least 50% or 75% of the mass, by weight, of a given sample. In one embodiment, these terms refer to the compound of the invention comprising at least 95, 96, 97, 98, 99 or 100%, of the mass, by weight, of a given sample. As used herein, the terms “purified”, “substantially purified,” and “isolated” when referring to a nucleic acid or protein, also refers to a state of purification or concentration different than that which occurs naturally, for example in a prokaryotic or eukaryotic environment, like, for example in a bacterial or fungal cell, or in the mammalian organism, especially human body. Any degree of purification or concentration greater than that which occurs naturally, including (1) the purification from other associated structures or compounds or (2) the association with structures or compounds to which it is not normally associated in said prokaryotic or eukaryotic environment, are within the meaning of “isolated”. The nucleic acid or protein or classes of nucleic acids or proteins, described herein, may be isolated, or otherwise associated with structures or compounds to which they are not normally associated in nature, according to a variety of methods and processes known to those of skill in the art.

In the context of the descriptions provided herein and of the appended claims, the use of “or” means “and/or” unless stated otherwise.

Similarly, “comprise,” “comprises,” “comprising”, “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “one or more” or the similar term “at least one” refers to e.g., 1, 2, 3, 4, 5, 6, 7, 8, 9, 10 or more.

When the lower and upper limits of a numerical range are disclosed, any numerical value and any inclusive range falling within that range is specifically disclosed, including its upper and lower end value. In particular, every range of values disclosed herein should be understood to mean every value and narrower range that falls within the broader range.

The term “about” indicates a potential variation of ±25% of the stated value, in particular ±15%, ±10%, more particularly ±5%, ±2% or ±1%.

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 100%, particularly in the range of 75 to 99.9%; more particularly 85 to 98.5%, like 95 to 99%.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### B.2 Chemical Definitions

The term halogen denotes in each case a fluorine, bromine, chlorine or iodine radical, in particular a fluorine radical.

“Alkyl” relates to a straight-chain or branched alkyl group having from 1 to 6, in particular 1 to 4 or 1, 2 or 3 carbon atoms. Examples include methyl, C₁-C₄-alkyl residues, such as methyl, ethyl, n-propyl, iso-propyl, n-butyl, 2-butyl, iso-butyl or tert-butyl; n-pentyl, 1-methylbutyl, 2-methylbutyl, 3-methylbutyl, 2,2-dimethylpropyl, 1-ethylpropyl, 1,1-dimethylpropyl, 1,2-dimethylpropyl; n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

“Lower alkyl” relates to a straight-chain or branched alkyl group having 1, 2, 3 or 4, in particular 1 or 2 carbon atoms. Examples include methyl, ethyl, n-propyl, iso-propyl, n-butyl, 2-butyl, iso-butyl or tert-butyl.

“Alkenyl” relates to a mono-unsaturated hydrocarbon radical comprising a single chemical carbon-carbon double bond, having 2, 3, 4, 5 or 6 carbon atoms, e.g. vinyl, allyl (2-propen-1-yl), 1-propen-1-yl, 2-propen-2-yl, methallyl (2-methylprop-2-en-1-yl) and the like.

“Lower alkenyl” relates to a mono-unsaturated hydrocarbon radical comprising a single chemical carbon-carbon double bond, having 2, 3 or 4 carbon atoms, e.g. vinyl, allyl (2-propen-1-yl), 1-propen-1-yl, 2-propen-2-yl, methallyl (2-methylprop-2-en-1-yl) and the like.

“Alkinyl” and “lower alkinyl” relate to the analogs of the abovementioned alkenyl or lower alkenyl groups and represent to a mono-unsaturated hydrocarbon radical comprising a single chemical carbon-carbon triple bond.

“Alkylene” relates to a straight-chain or branched alkylene group having from 1 to 6, in particular 1 to 4 carbon atoms. Examples include methylene, ethylene, 1,2-ethylene, 1,3-propylene, isopropylene; 1-4-butylene, 1-5-pentylene 1-6-hexylene and the respective branched analogues thereof.

“Lower alkylene” relates to a straight-chain or branched alkylene group having from 1 to 4 carbon atoms. Examples include methylene, ethylene, 1,2-ethylene, 1,3-propylene, isopropylene, 1-4-butylene and the respective branched analogues thereof.

“Alkoxy” relates to a radical of the formula R—O—, wherein R is a straight-chain or branched alkyl group as defined above having from 1 to 6, in particular 1 to 4 or 1 to 3 carbon atoms as defined herein. Non limiting examples are methoxy, ethoxy, n-propoxy, iso-propoxy, n-butoxy, 2-butoxy, iso-butoxy or tert-butoxy.

“Alkyleneoxy” relates to a radical of the formula —R—O—, wherein R is a straight-chain or branched alkylene group having from 1 to 6, in particular 1 to 4 or 1 to 3 carbon atoms as defined herein.

“Lower alkyleneoxy” relates to a radical of the formula —R—O—, wherein R is a straight-chain or branched lower alkylene group having from 1 to 4 or 1 to 3 carbon atoms as defined herein. Examples include methyleneoxy, ethyleneoxy, 1,2-ethyleneoxy, 1,3-propyleneoxy, isopropyleneoxy and 1-4-butyleneoxy.

“Polyalkyleneoxy” relates to a moiety comprising at least two, as for example 2 to 20, 2 to 15, 2 to 10 or 2 to 5 repetitive units of covalently linked, identical or different, in particular identical, lower alkyleneoxy groups having at least two carbon atoms, as defined above, in particular polyethyleneoxy and polypropyleneoxy groups, having 2 to 20, 2 to 15, 2 to 10 or 2 to 5 identical repetitive units.

“Alkenoxy” relates to a radical of the formula R—O—, wherein R is a straight-chain or branched alkenyl group as having from 1 to 6, in particular 1 to 4 or 1 to 3 carbon atoms as defined herein.

“Alkanoyloxy” relates to a radical of the formula R—(CO)—O—, wherein R is a straight-chain or branched alkyl group having from 1 to 6, in particular 1 to 4 or 1 to 3 carbon atoms as defined herein.

“Alkylaminocarbonyloxy” relates to a radical of the formula R—NH—(CO)—O—, wherein R is a straight-chain or branched alkyl group having from 1 to 6, in particular 1 to 4 or 1 to 3 carbon atoms as defined herein.

“Alkylthio” relates to a radical of the formula R—S—, wherein R is an alkyl radical having from 1 to 4, preferably from 1 to 3 carbon atoms as defined herein.

“Alkylamino” relates to a radical of the formula R—NH— wherein R is an alkyl radical having from 1 to 6, in particular from 1 to 4 carbon atoms as defined herein. Examples include methylamino, ethylamino, n-propylamino, iso-propylamino, n-butylamino, 2-butylamino, iso-butylamino, tert-butylamino and the like.

“Dialkylamino” relates to a radical of the formula RR′N— wherein R and R′ are independently of each other an alkyl radical having from 1 to 6, in particular from 1 to 4 carbon atoms as defined herein. Examples include dimethylamino, diethylamino, N-methyl-N-ethylamino and the like.

“Alkenylamino” relates to a radical of the formula R—NH— wherein R is an alkenyl radical having from 2 to 6, in particular from 2 to 4 carbon atoms as defined herein. Examples include vinylamino, allylamino (2-propen-1-yl-amino), 1-propen-1-yl-amino, 2-propen-2-yl-amino, methallylamino (2-methylprop-2-en-1-yl-amino) and the like.

“N-Alkyl-N-alkenylamino” relates to a radical of the formula RR′N— wherein R is an alkyl radical having from 1 to 6, in particular from 1 to 4 carbon atoms as defined herein and R′ an alkenyl radical having from 2 to 6, in particular from 2 to 4 carbon atoms as defined herein. Examples include N-methyl-N-vinylamino, N-methyl-N-allylamino (N-methyl-N-2-propen-1-yl-amino), N-methyl-N-1-propen-1-yl-amino, N-methyl-N-2-propen-2-yl-amino, N-methyl-N-methallylamino (N-methyl-N-2-methylprop-2-en-1-yl-amino) and the like.

“Dialkenylamino” relates to a radical of the formula RR′N— wherein R and R′ are independently of each other an alkyl radical having from 2 to 6, in particular from 2 to 4 carbon atoms as defined herein. Examples include divinylamino, diallylamino (di-(2-propen-1-yl)-amino), N-vinyl-N-allyl-amino and the like.

“Aryl” relates to monovalent mono- or polycyclic aromatic moieties, in particular having 6 to 14 ring carbon atoms, in particular, phenyl, fluorenyl, naphthenyl and phenanthrenyl

“Arylene” relates to the bivalent analog of the above-mentioned aryl groups, in particular 1,2-, 1,3- and 1,4-phenylene,

“Halogen” relates to F, Cl, Br or I;

Unless indicated otherwise, the term “substituents” are selected from halogen, C₁-C₄-alkyl, CN, CF₃, hydroxyl, —O—CF₃, C₁-C₄-alkoxy, C₂-C₄-alkanoyloxy, C₁-C₄-alkylaminocarbonyloxy and C₁-C₄-alkylthio; carboxy and carboxy-C₁-C₄-alkyl.

Unless indicated otherwise, the term “substituted” means that a radical is substituted with 1, 2 or 3, especially 1 or 2, substituent(s).

A “linkage” is formed between two neighbored structural motifs of a compound of the invention and is, unless otherwise indicated, either a chemical bond, or is selected from a ether, thioether, ester, amide, carbamate, dicarbamate, carbonate, hydrazine, urea, alkylene oxide or linear or branched polyalkylene oxide linkage in any possible orientation.

An “ether” linkage contains at least one group of the type: (—O—).

A “thioether” linkage contains at least one group of the type: (—S—).

An “amide” linkage contains at least one group of the type: —C(═O)N(R)— or —(R)N—C(═O)—.

A “carbamate” linkage contains at least one group of the type: —O—C(═O)—N(R)— or —N(R)—C(═O)—O—.

A “dicarbamate” linkage contains at least one group of the type:

—N(R)—C(═O)O—R′—OC(═O)—N(R)—

A “carbonate” linkage contains at least one group of the type: —C(═O)O—, —O—C(═O)—, —O—C(═O)O— or —O—C(═O)—O—.

A “hydrazine” linkage contains at least one group of the type: —NH—NH—

An “urea” linkage contains at least one group of the type: —N(R)—C(═O)—N(R)—

An “alkylene oxide” linkage contains at least one group of the type: —((CH₂)ₙ—O)— or —(O—(CH₂)ₙ)—, with n=1, 2, 3 or 4, in particular 1 or 2.

A “polyalkylene oxide” linkage contains repetitive units of same or different alkylene oxides groups as defined above and may be linear or branched, in particular linear; as for example —((CH₂)ₙ—O)ₘ— or —(O—(CH₂)ₙ)ₘ— with n=1, 2, 3 or 4, in particular 1 or 2; and m=2 to 20, 2 to 15, 2 to 10 or 2 to 5.

In the above-mentioned chemical formulae of particular linkages residues R independently of each other may represent H or lower alkyl, lower alkenyl or lower alkenyl, in particular methyl, or ethyl; R′ represents a lower alkylene group or lower alkenylene group; in particular methylene or ethylene.

A “cleavable group” encompasses any group, which may be cleaved enzymatically or chemically, in particular under in vivo or ex vivo conditions; an enzymatic cleavage may be effected, for example, through the action of a protease; a chemical cleavage, may be effected for example through hydrolytic cleavage or reductive cleavage of S—S bonds.

A “tetrazine” group according to the present invention represents, unless otherwise defined, a residue that consists of a six-membered aromatic ring containing four nitrogen atoms with the molecular formula —C₂N₄—, in particular derived from the 1,2,4,5-tetrazine or s-tetrazine isomer, and liked to neighboring groups via ring carbon positions 3 and 6.

A “tetrazine-reactive group” is a chemical moiety that has the ability to chemically react, in particular, via a so-called “bioorthogonal” or “click reaction”, with a tetrazine group as defined herein. In particular, such tetrazine reactive group is selected from dienophiles. More particularly, it is selected from dienophiles having the ability to react in a biological environment with the tetrazine group. As non-limiting examples there may be mentioned, isonitrile groups, norbornene groups, bicyclononynyl groups, cyclooctenyl groups, cyclooctinyl groups, cyclopropenyl groups, cyclobutynyl groups, and spirohexenyl groups and their stereoisomers, alkene or allyl groups or dihydro azete groups.

“Tetrazine ligation” refers to the reaction of a trans-cyclooctene and an s-tetrazine in an inverse-demand Diels Alder reaction followed by retro Diels Alder reaction to eliminate nitrogen (N₂). A reaction of this type proceeds with high velocity, allowing bio molecule modification at extremely low concentrations.

Compounds as herein described may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. All stereoisomers, diastereomers, Z- and E-forms, in purified and mixture forms are included. Accordingly, when a compound is recited by specific name or a class of compounds is recited, all these forms are intended to be included.

Compounds as herein described may also exist in more than one form of structural isomers also designated as constitutional isomers or regioisomers. These are molecules that differ only in the different sequence of their atoms or atomic groups while having the same gross formula.

Therefore, unless otherwise stated, for each of the compounds, biomolecules and conjugates as described herein, any such potential stereo- or regiosomeric form or mixture of more than one stereo- and/or regiosomeric form is within the scope of the present invention.

An “inverse electron-demand Diels-Alder (IEDDA) cycloaddition” is a reaction between an electron-poor diene and an electron-rich dienophile and represents only one example of different types of “bioorthogonal reactions”. The diene used may be a 1,2,4,5-tetrazine or a 1,2,4-triazine. The dienophiles encompass a variety of molecules including strained cyclic alkenes, such as trans-cyclooctenes (TCO, norbornenes, cyclopropenes or azetines). Of these, the reaction between a tetrazine and TCO is the fastest reported to date and suitable for in vivo applications (Smeek et al, Current Opinion in Chemical Biology Volume 60, February 2021, Pages 79-88).

The term “bioorthogonal” refers to any chemical reaction that can occur inside of living systems, i.e. in aqueous environment, without interfering with native biochemical processes. “Tetrazine ligation” may for example be mentioned as one type of bioorthogonal reaction. Bioorthogonal chemistry typically proceeds in two steps. First, a cellular substrate is modified with a bioorthogonal functional group (also designated chemical reporter) as for example one of the above-identified “tetrazine reactive groups”. Cellular substrates include for example immunoglobulins, like natural or recombinant antibodies, etc. The chemical reporter must not alter the structure of the substrate dramatically to avoid affecting its bioactivity. In a second step, a probe containing the complementary functional group, as for example a tetrazine group as described herein, is introduced to react and label the substrate.

“Acid or base addition salts” of compounds of the invention are especially addition salts with physiologically tolerated acids or bases. Physiologically tolerated acid addition salts can be formed by treatment of the base form of a compound of the invention with appropriate organic or inorganic acids. Compounds of the invention containing an acidic proton may be converted into their non-toxic metal or amine addition salt forms by treatment with appropriate organic and inorganic bases. The compounds and salts of the invention also comprise the hydrates and solvent addition forms thereof, e.g. hydrates, alcoholates and the like.

“Physiologically tolerated” acids or bases are in particular those which are tolerated by the system used for the incorporation of the first and second dienophiles (e.g. a biological system such as a translation system used for preparation of polypeptides with trans-cyclooctenyl or cyclooctynyl groups), e.g. which are substantially non-toxic to living cells.

A “pharmaceutical composition” comprises in addition to an ADC of the invention one or more substances such as selected from the group consisting of pharmaceutically acceptable preservatives, pharmaceutically acceptable colorants, pharmaceutically acceptable protective colloids, pharmaceutically acceptable pH regulators and pharmaceutically acceptable osmotic pressure regulators. Such substances are described in the art. A more detailed description of pharmaceutical compositions of the invention is provided below.

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

### B.3 Biochemical Definitions

A “polypeptide” is any oligomer of amino acid residues (natural or unnatural, or a combination thereof), of any length, typically but not exclusively joined by covalent peptide bonds. A polypeptide can be from any source, e.g., a naturally occurring polypeptide, a polypeptide produced by recombinant molecular genetic techniques, a polypeptide from a cell or translation system, or a polypeptide produced by cell-free synthetic means. A polypeptide is characterized by its amino acid sequence, e.g., the primary structure of its component amino acid residues. As used herein, the amino acid sequence of a polypeptide is not limited to full-length sequences, but can be partial or complete sequences. Furthermore, it is not intended that a polypeptide be limited by possessing or not possessing any particular biological activity.

As used herein, the term “protein” is synonymous with polypeptide. The term “peptide” refers to a small polypeptide, for example but not limited to, from 2-25 amino acids in length.

A protein having incorporated into its amino acid sequence at least one ncAA in a particular embodiment is utilized to form a “targeting agent”. The primary object of such targeting agent is the formation of a covalent or noncovalent linkage with a particular “target”. A secondary object of the targeting agent is the targeted transport of a “payload molecule” to said target. In order to achieve said second object said targeting agent has to be combined (reversibly or irreversibly) with at least one “payload molecule”. For this purpose said targeting agent is functionalised by said at least one ncAA. The functionalized targeting agent carrying said at least one ncAA, may then be linked to said at least one payload molecule through bioconjugation via said ncAA residue. Said ncAA is reactive with the payload molecule which in turn carries a corresponding moiety, in the present case a particular tetrazine moiety, reactive with said ncAA residue of the targeting agent. The thus obtained bioconjugate allows the transfer of the payload molecule to the intended target.

As used herein, the term “to incorporate an unnatural amino acid”, e.g., into a targeting polypeptide, refers to the direct addition of an unnatural amino acid to a growing polypeptide chain during primary construction of the target polypeptide, e.g., via translation or chemical synthesis.

An unnatural amino acid (“UNAA”) can be directly incorporated into targeting polypeptides using any of a number of methods known in the art. While many embodiments utilize orthogonal translation systems as the route of direct incorporation of unnatural amino acids, other direct incorporation methods (e.g., in vitro translation systems, solid-phase synthesis, etc.) can be used alternatively. It will be appreciated that in typical embodiments herein, an unnatural amino acid is preferably incorporated into target polypeptide, i.e., during construction of the polypeptide, and is not added via post-translational chemical derivatization.

In certain embodiments described herein, the unnatural amino acids can be site-specifically incorporated into a targeting polypeptide with high efficiency and high fidelity using “orthogonal tRNA/aminoacyl-tRNA synthetase pairs”.

The term “translation system” refers to the components necessary to incorporate an amino acid in a growing polypeptide chain (protein). Components of a translation system can include, e.g., ribosomes, tRNAs, synthetases, mRNA and the like. The translation system may be an in vivo or an in vitro translation system.

An “in vitro translation system” may be a cell-free translation system. A cell-free translation system is a system for synthesizing a desired protein by obtaining protein factors required for mRNA translation, e.g., in form of a cell extract, followed by reconstituting this reaction in vitro. Such cell-free systems and their use for protein synthesis are known in the art. Examples include extracts of E. coli, wheat germ extract, or rabbit reticulocyte lysate (Spirin and Swartz, Cell-free Protein Synthesis, Wiley VCH Verlag, Weinheim, Germany, 2008).

An aminoacyl tRNA synthetase (RS) is an enzyme capable of acylating a tRNA with an amino acid or amino acid analog. Expediently, the RS used in the methods of the invention is capable of acylating a tRNA with an unnatural amino acid.

The methods of the invention expediently utilize a “tRNA/aminoacyl tRNA synthetase (tRNA/RS) pair”. Preferably, the tRNA/RS pair used in the processes of the invention is orthogonal to the translation system.

The term “orthogonal” as used herein refers to a molecule (e.g., an orthogonal tRNA (O-tRNA) and/or an orthogonal aminoacyl tRNA synthetase (O—RS)) that is used with reduced efficiency by a translation system of interest (e.g., a cell). Orthogonal refers to the inability or reduced efficiency, e.g., less than 20% efficient, less than 10% efficient, less than 5% efficient, or e.g., less than 1% efficient, of an orthogonal tRNA or an orthogonal aminoacyl tRNA synthetase to function with the endogenous aminoacyl tRNA synthetases or endogenous tRNAs of the translation system of interest. For example, an orthogonal tRNA in a translation system of interest is acylated by any endogenous aminoacyl tRNA synthetase of a translation system of interest with reduced or even zero efficiency, when compared to acylation of an endogenous tRNA by the endogenous aminoacyl tRNA synthetase. In another example, an orthogonal aminoacyl tRNA synthetase acylates any endogenous tRNA in the translation system of interest with reduced or even zero efficiency, as compared to acylation of the endogenous tRNA by an endogenous aminoacyl tRNA synthetase.

Orthogonal tRNA/RS pairs used in processes of the invention preferably have following properties: the O-tRNA is preferentially acylated with the unnatural amino acid of the invention by the O—RS. In addition, the orthogonal pair functions in the translation system of interest, e.g., the translation system uses the unnatural amino acid acylated O-tRNA to incorporate the unnatural amino acid of the invention in a polypeptide chain. Incorporation occurs in a site specific manner, e.g., the O-tRNA recognizes a selector codon, e.g., an amber stop codon, in the mRNA coding for the polypeptide.

The term “preferentially acylates” refers to an efficiency of, e.g., about 50% efficient, about 70% efficient, about 75% efficient, about 85% efficient, about 90% efficient, about 95% efficient, or about 99% or more efficient, at which an O—RS acylates an O-tRNA with an unnatural amino acid compared to an endogenous tRNA or amino acid of a translation system of interest. The unnatural amino acid is then incorporated in a growing polypeptide chain with high fidelity, e.g., at greater than about 75% efficiency for a given selector codon, at greater than about 80% efficiency for a given selector codon, at greater than about 90% efficiency for a given selector codon, at greater than about 95% efficiency for a given selector codon, or at greater than about 99% or more efficiency for a given selector codon.

The term “selector codon” refers to codons recognized by the O-tRNA in the translation process and not recognized by an endogenous tRNA. The O-tRNA anticodon loop recognizes the selector codon on the mRNA and incorporates its amino acid, e.g., an unnatural amino acid, at this site in the polypeptide. Selector codons can include, e.g., nonsense codons, such as stop codons, e.g., amber, ochre, and opal codons; four or more base codons; codons derived from natural or unnatural base pairs and the like. For a given system, a selector codon can also include one of the natural three base codons (i.e. natural triplets), wherein the endogenous system does not use said natural triplet, e.g., a system that is lacking a tRNA that recognizes the natural triplet or a system wherein the natural triplet is a rare codon.

An “anticodon” has the reverse complement sequence of the corresponding codon.

An O-tRNA/O—RS pair is composed of an O-tRNA, e.g., a suppressor tRNA, or the like, and an O—RS.

A “suppressor tRNA” is a tRNA that alters the reading of a messenger RNA (mRNA) in a given translation system. A suppressor tRNA can read through, e.g., a stop codon, a four base codon, or a rare codon.

The O-tRNA is not acylated by endogenous synthetases and is capable of decoding a selector codon, as described herein.

The O—RS recognizes the O-tRNA, e.g., with an extended anticodon loop, and preferentially acylates the O-tRNA with an unnatural amino acid.

The tRNA and the RS used in the processes of the invention can be naturally occurring or can be derived by mutation of a naturally occurring tRNA and/or RS from a variety of organisms. In various embodiments, the tRNA and RS are derived from at least one organism. In another embodiment, the tRNA is derived from a naturally occurring or mutated naturally occurring tRNA from a first organism and the RS is derived from naturally occurring or mutated naturally occurring RS from a second organism.

A suitable tRNA/RS pair may be selected from libraries of mutant tRNA and RS, e.g. based on the results of a library screening. Alternatively, a suitable tRNA/RS pair may be a heterologous tRNA/synthetase pair that is imported from a source species into the translation system. Preferably, the cell used as translation system is different from said source species.

For example a suitable orthogonal O-tRNA can be derived from an archaebacterium, such as Methanococcus jannaschii, Methanobacterium thermoautotrophicum, Halobacterium such as Haloferax volcanii and Halobacterium species NRC—I, Archaeoglobus fulgidus, Pyrococcus furiosus, Pyrococcus horikoshii, Aeuropyrum pemix, Methanococcus maripaludis, Methanopyrus kandleri, Methanosarcina mazei (Mm), Pyrobaculum aerophilum, Pyrococcus abyssi, Sulfolobus solfataricus (Ss), Sulfolobus tokodaii, Thermoplasma acidophilum, Thermoplasma volcanium, or the like, or a eubacterium, such as Escherichia coli, Thermus thermophilus, Bacillus subtilis, Bacillus stearothermphilus, or the like, while the orthogonal O—RS can be derived from an organism or combination of organisms, e.g., an archaebacterium, such as Methanococcus jannaschii, Methanobacterium thermoautotrophicum, Halobacterium such as Haloferax volcanii and Halobacterium species NRC-J, Archaeoglobus fulgidus, Pyrococcus furiosus, Pyrococcus horkoshii, Aeuropyrum pemix, Methanococcus maripaludis, Methanopyrus kandleri, Methanosarcina mazei, Methanosarcina bakeri; Methanosarcina hafniense; Pyrobaculum aerophilum, Pyrococcus abyssi, Sulfolobus solfataricus, Sulfolobus tokodaii, Thermoplasma acidophilum, Thermoplasma volcanium, or the like, or a eubacterium, such as Escherichia coli, Thermus thermophilus, Bacillus subtilis, Bacillus stearothermophilus, or the like. In one embodiment, eukaryotic sources, e.g., plants, algae, protists, fungi, yeasts, animals, e.g., mammals, insects, arthropods, or the like can also be used as sources of O-tRNAs and O-RSs

Methods for evolving tRNA/RS pairs are described, e.g., in WO 02/085923 and WO 02/06075.

Preferably, the RS is a pyrrolysyl tRNA synthetase (pyIRS) capable of acylating a tRNA with the unnatural amino acid of the invention. The pyrrolysyl tRNA synthetase used in methods of the invention may be a wildtype or a genetically engineered pyIRS. Examples for wildtype pyIRS include, but are not limited to pyIRS from archaebacteria and eubacteria such as Methanosarcina mazei, Methanosarcina barkeri, Methanococcoides burtonii, Methanosarcina acetivorans, Methanosarcina thermophila, and Desulitobacterium hafniense.

Pyrrolysyl tRNA synthetase (PyIRS) is an aminoacyl tRNA synthetase (RS). RSs are enzymes capable of acylating a tRNA with an amino acid or amino acid analog. Expediently, the PyIRS of the invention is enzymatically active, i.e. is capable of acylating a tRNA (tRNA^{PyI}) with a certain amino acid or amino acid analog, preferably with an UNAA or salt thereof

The term “archaeal pyrrolysyl tRNA synthetase” (abbreviated as “archaeal PyIRS”) as used herein refers to a PyIRS, wherein at least a segment of the PyIRS amino acid sequence, or the entire PyIRS amino acid sequence, has at least 60%, at least 70%, at least 80%, at least 90%, at least 95%, at last 99%, or 100% sequence identity to the amino acid sequence of a naturally occurring PyIRS from an archaeon, or to the amino acid sequence of an enzymatically active fragment of such naturally occurring PyIRS.

The PyIRS of the present invention may comprise a mutant archaeal PyIRS, or an enzymatically active fragment thereof.

Generally, “mutant archaeal PyIRSs” or “mutated archaeal PyIRSs” differ from the corresponding wildtype PyIRSs in comprising additions, substitutions and/or deletions of one or more than one amino acid residue. Preferably, these are modifications which improve PyIRS stability, alter PyIRS substrate specificity and/or enhance PyIRS enzymatic activity. Particularly preferred “mutant archaeal PyIRSs” or “mutated archaeal PyIRSs” are described in more detail herein below.

The term “nuclear export signal” (abbreviated as “NES”) refers to an amino acid sequence which can direct a polypeptide containing it (such as a NES-containing PyIRS of the invention) to be exported from the nucleus of a eukaryotic cell. Said export is believed to be mostly mediated by Crm1 (chromosomal region maintenance 1, also known as karyopherin exportin 1). NESs are known in the art. For example, the database ValidNESs (http://validness.ym.edu.tw/) provides sequence information of experimentally validated NES-containing proteins. Further, NES databases like, e.g., NESbase 1.0 (www.cbs.dtu.dk/databased/NESbase-1.0/; see Le Cour et al., Nucl Acids Res 31(1), 2003) as well as tools for NES prediction like NetNES (www.cbs.dtu.dk/services/NetNES/; see La Cour et al., La Cour et al., Protein Eng Des Sel 17(6):527-536, 2004), NESpredictor (NetNES, http://www.cbs.dtu.dk/; see Fu et al., Nucl Acids Res 41:D338-D343, 2013; La Cour et al., Protein Eng Des Sel 17(6):527-536, 2004)) and NESsential (a web interface combined with ValidNESs) are available to the public. Hydrophobic leucine-rich NESs are most common and represent the best characterized group of NESs to date. A hydrophobic leucine-rich NES is a non-conservative motif having 3 or 4 hydrophobic residues. Many of these NESs comprise the conserved amino acid sequence pattern LxxLxL (SEQ ID NO:111) or LxxxLxL (SEQ ID NO:112), wherein each L is independently selected from leucine, isoleucine, valine, phenylalanine and methionine amino acid residues, and each x is independently selected from any amino acid (see La Cour et al., Protein Eng Des Sel 17(6):527-536, 2004).

The term “nuclear localization signal” (abbreviated as “NLS”, also referred to in the art as “nuclear localization sequence”) refers to an amino acid sequence which can direct a polypeptide containing it (e.g., a wild-type archaeal PyIRS) to be imported into the nucleus of a eukaryotic cell. Said export is believed to be mediated by binding of the NLS-containing polypeptide to importin (also known as karyopherin) so as to form a complex that moves through a nuclear pore. NLSs are known in the art. A multitude of NLS databases and tools for NLS prediction are available to the public, such as NLSdb (see Nair et al., Nucl Acids Res 31(1), 2003), cNLS Mapper (www.nls-mapper.aib.keio.ac.jp; see Kosugi et al., Proc Natl Acad Sci USA. 106(25):10171-10176, 2009; Kosugi et al., J Biol Chem 284(1):478-485, 2009), SeqNLS (see Lin et al., PLoS One 8(10):e76864, 2013), and NucPred (www.sbc.su.se/˜maccallr/nucpred/; see Branmeier et al., Bioinformatics 23(9):1159-60, 2007).

Mutant archaeal PyIRSs of the invention as defined above can be further modified by removing the NLS optionally present in said naturally occurring PyIRS where the mutant is derived from and/or by introducing at least one NES. The NLS in the naturally occurring PyIRS can be identified using known NLS detection tools such as, e.g., cNLS Mapper.

The removal of a NLS from and/or the introduction of a NES into an archaeal PyIRS or mutant thereof, can change the localization of the thus modified polypeptide when expressed in a eukaryotic cell, and in particular can avoid or reduce accumulation of the polypeptide in the nucleus of the eukaryotic cell. Thus, the localization of a PyIRS mutant of the invention expressed in a eukaryotic cell can be changed compared to a PyIRS or PyIRS mutant, which differs from the PyIRS mutant of the invention in that it (still) comprises the NLS and lacks the NES.

Where the archaeal PyIRS of the invention comprises a NES but (still) comprises an NLS, the NES is preferably chosen such that the strength of the NES overrides the NLS preventing an accumulation of the PyIRS in the nucleus of a eukaryotic cell.

Removal of the NLS from a wild-type or mutant PyIRS and/or introduction of a NES into the wild-type or mutant PyIRS so as to obtain a PyIRS of the invention do not abrogate PyIRS enzymatic activity. Preferably, PyIRS enzymatic activity is maintained at basically the same level, i.e. the PyIRS of the invention has at least 50%, at least 60%, at least 70%, at least 80%, at least 90%, or at least 91, 92, 93, 94, 95, 96, 97, 98 or 99% of the enzymatic activity of the corresponding wild-type or mutant PyIRS.

The NES is expediently located within the PyIRS or mutant PyIRS of the invention such that the NES is functional. For example, a NES can be attached to the C-terminus (e.g., C-terminal of the last amino acid residue) or the N-terminus (e.g., in between amino acid residue 1, the N-terminal methionine, and amino acid residue 2) of a wild-type or mutant archaeal PyIRS.

The disclosure of WO2018/06948 disclosing mutated PyIRSs modified by the incorporation of NES and/deletion of NLS sequences is herewith explicitly referred to and incorporated by reference.

### C. Particular Embodiments

The tetrazine compounds of the invention described herein below having the general formula (I) are used for preparation of conjugates, in particular bioconjugates. For this purpose, a compound of formula (I) is reacted via its functional tetrazine moiety with a suitable tetrazine-reactive second functional group carried by a conjugation partner, as for example a biomolecule, in particular a targeting molecule, like for example an antibody molecule.

Said tetrazine-reactive functional group may for example be a cyclooctynyl or trans-cyclooctenyl group.

The invention provides processes for preparing such polypeptides, in vivo or in vitro. In particular, said tetrazine-reactive functional group can be translationally incorporated in a polypeptide that is encoded by a polynucleotide comprising one or more than one selector codon(s).

The present invention relates to the following main aspects and particular embodiments thereof.

### 1. The First Aspect of the Present Invention

A first aspect of the present invention relates to payload molecules functionalized by means of a particular tetrazine group, which are adapted to conjugation with a second molecule, in particular a bio-molecule, carrying a functional counterpart group reactive with said tetrazine group of the functionalized payload molecule.

According to a first embodiment of the invention a tetrazine-functionalized compound of the following general formula I is provided,

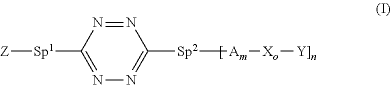

wherein
m is 0 or 1
n represents an integer selected from 1 and 2
o is 0 or represents an integer selected from 1 or 2
A represents a cleavable moiety
Sp¹ and Sp² independently of each other represent a spacer moiety
X represents a self-immolative moiety
Y represents a payload residue (or cargo) and
Z represents a hydrophilic group.

According to a particular embodiment m is 0.

According to another particular embodiment m as 1.

According to another particular embodiment n is 1.

According to another particular embodiment n is 2.

According to another particular embodiment o is 0.

According to another particular embodiment o is 1.

According to another particular embodiment o is 2.

According to another particular embodiment Sp¹ and Sp² are different.
according to another particular embodiment Sp¹ and Sp² are identical.

Particular examples of parameter combinations of m, n and o as well as Sp¹ and Sp² are:

| No. | m | n | o | Sp1/Sp2 |
|---|---|---|---|---|
| 1 | 0 | 1 | 0 | identical |
| 2 | 0 | 2 | 0 | identical |
| 3 | 0 | 1 | 1 | identical |
| 4 | 0 | 2 | 1 | identical |
| 5 | 0 | 1 | 2 | identical |
| 6 | 0 | 2 | 2 | identical |
| 7 | 1 | 1 | 0 | identical |
| 8 | 1 | 2 | 0 | identical |
| 9 | 1 | 1 | 1 | identical |
| 10 | 1 | 2 | 1 | identical |
| 11 | 1 | 1 | 2 | identical |
| 12 | 1 | 2 | 2 | identical |
| 13 | 0 | 1 | 0 | different |
| 14 | 0 | 2 | 0 | different |
| 15 | 0 | 1 | 1 | different |
| 16 | 0 | 2 | 1 | different |
| 17 | 0 | 1 | 2 | different |
| 18 | 0 | 2 | 2 | different |
| 19 | 1 | 1 | 0 | different |
| 20 | 1 | 2 | 0 | different |
| 21 | 1 | 1 | 1 | different |
| 22 | 1 | 2 | 1 | different |
| 23 | 1 | 1 | 2 | different |
| 24 | 1 | 2 | 2 | different |

According to a second embodiment of the invention, residue Z in said compound general formula I of the first embodiment is selected from one of the following hydrophilic groups:
a) phosphor and/or sulfur containing hydrophilic moieties Z,
in particular (R¹O)₂P(O)—, (R^{1a}O)₂P(O)—O—, (R²O)₃P—O—, R³S(O)₂—, (R⁴O)S(O)₂O—, or (R^{4a}O)S(O)₂—,
   wherein
   residues R¹ to R⁴, R^{1a} and R^{4a} are same or different and independently of each other represent H or lower alkyl, in particular methyl or ethyl;
   or a salt form, as for example a physiologically tolerated salt, of said phosphor and/or sulfur containing hydrophilic moieties;
b) linear or branched mono- or poly-alkylene oxide moieties Z, in particular selected from linear the moieties —((CH₂)ₓ—O)_{y}—R⁵, —(O—(CH₂)ₓ)_{y}—H and —(O—(CH₂)ₓ)_{y}—OR⁶, and the branched analogues thereof;
   wherein
   residues R⁵ and R⁶ independently of each other represent H or lower alkyl, in particular H, methyl or ethyl;
   x independently of each other represent an integer selected from 1, 2, 3 or 4; in particular 1 or 2; and
   y independently of each other represent an integer from 1 to 20, in particular 1 to 15, 1 to 10 or 1 to 4; as for example 1, 2, 3, or 4

### As Regards Group a):

According to a particular embodiment Z is (R¹O)₂P(O)—, (R^{1a}O)₂P(O)—O— or (R²O)₃P—O— and more particularly (R¹O)₂P(O)—;
wherein
   residues R¹; R² and R^{1a} are same or different independently of each other represent H or methyl or ethyl;

### As Regards Group b):

According to another particular embodiment Z is a linear mono- or poly-alkylene oxide moiety.

According to still another particular embodiment Z is selected from linear the moieties —((CH₂)ₓ—O)_{y}—R⁵, —(O—(CH₂)ₓ)_{y}—H and —(O—(CH₂)ₓ)_{y}—OR⁶, wherein residues R⁵ and R⁶ independently of each other represent H, methyl or ethyl;

According to still another particular embodiment x independently of each other represent therein an integer selected from 1 or 2.

Particular examples of parameter combinations of parameters Z, m, n and o as well as Sp¹ and Sp² are:

| | | | | | |
|---|---|---|---|---|---|
| No. | m | n | o | Sp1/Sp2 | Z |

| | | | | |
|---|---|---|---|---|
| 0 | 1 | 0 | identical | (R¹ O)₂P(O)— |
| 0 | 2 | 0 | identical | (R¹ O)₂P(O)— |
| 0 | 1 | 1 | identical | (R¹ O)₂P(O)— |
| 0 | 2 | 1 | identical | (R¹ O)₂P(O)— |
| 0 | 1 | 2 | identical | (R¹ O)₂P(O)— |
| 0 | 2 | 2 | identical | (R¹ O)₂P(O)— |
| 1 | 1 | 0 | identical | (R¹ O)₂P(O)— |
| 1 | 2 | 0 | identical | (R¹ O)₂P(O)— |
| 1 | 1 | 1 | identical | (R¹ O)₂P(O)— |
| 1 | 2 | 1 | identical | (R¹ O)₂P(O)— |
| 1 | 1 | 2 | identical | (R¹ O)₂P(O)— |
| 1 | 2 | 2 | identical | (R¹ O)₂P(O)— |
| 0 | 1 | 0 | different | (R¹ O)₂P(O)— |
| 0 | 2 | 0 | different | (R¹ O)₂P(O)— |
| 0 | 1 | 1 | different | (R¹ O)₂P(O)— |
| 0 | 2 | 1 | different | (R¹ O)₂P(O)— |
| 0 | 1 | 2 | different | (R¹ O)₂P(O)— |
| 0 | 2 | 2 | different | (R¹ O)₂P(O)— |
| 1 | 1 | 0 | different | (R¹ O)₂P(O)— |
| 1 | 2 | 0 | different | (R¹ O)₂P(O)— |
| 1 | 1 | 1 | different | (R¹ O)₂P(O)— |
| 1 | 2 | 1 | different | (R¹ O)₂P(O)— |
| 1 | 1 | 2 | different | (R¹ O)₂P(O)— |
| 1 | 2 | 2 | different | (R¹ O)₂P(O)— |

wherein R¹ is H or lower alkyl.
or

| No. | m | n | o | Sp1/Sp2 | Z | R¹ |
|---|---|---|---|---|---|---|
| | 0 | 1 | 0 | identical | (R¹ O)₂P(O)— | H |
| | 0 | 2 | 0 | identical | (R¹ O)₂P(O)— | H |
| | 0 | 1 | 1 | identical | (R¹ O)₂P(O)— | H |
| | 0 | 2 | 1 | identical | (R¹ O)₂P(O)— | H |
| | 0 | 1 | 2 | identical | (R¹ O)₂P(O)— | H |
| | 0 | 2 | 2 | identical | (R¹ O)₂P(O)— | H |
| | 1 | 1 | 0 | identical | (R¹ O)₂P(O)— | H |
| | 1 | 2 | 0 | identical | (R¹ O)₂P(O)— | H |
| | 1 | 1 | 1 | identical | (R¹ O)₂P(O)— | H |
| | 1 | 2 | 1 | identical | (R¹ O)₂P(O)— | H |
| | 1 | 1 | 2 | identical | (R¹ O)₂P(O)— | H |
| | 1 | 2 | 2 | identical | (R¹ O)₂P(O)— | H |
| | 0 | 1 | 0 | different | (R¹ O)₂P(O)— | H |
| | 0 | 2 | 0 | different | (R¹ O)₂P(O)— | H |
| | 0 | 1 | 1 | different | (R¹ O)₂P(O)— | H |
| | 0 | 2 | 1 | different | (R¹ O)₂P(O)— | H |
| | 0 | 1 | 2 | different | (R¹ O)₂P(O)— | H |
| | 0 | 2 | 2 | different | (R¹ O)₂P(O)— | H |
| | 1 | 1 | 0 | different | (R¹ O)₂P(O)— | H |
| | 1 | 2 | 0 | different | (R¹ O)₂P(O)— | H |
| | 1 | 1 | 1 | different | (R¹ O)₂P(O)— | H |
| | 1 | 2 | 1 | different | (R¹ O)₂P(O)— | H |
| | 1 | 1 | 2 | different | (R¹ O)₂P(O)— | H |
| | 1 | 2 | 2 | different | (R¹ O)₂P(O)— | H |

or

| No. | m | n | o | Sp1/Sp2 | Z | R¹ |
|---|---|---|---|---|---|---|
| | 0 | 1 | 0 | identical | (R¹ O)₂P(O)— | methyl |
| | 0 | 2 | 0 | identical | (R¹ O)₂P(O)— | methyl |
| | 0 | 1 | 1 | identical | (R¹ O)₂P(O)— | methyl |
| | 0 | 2 | 1 | identical | (R¹ O)₂P(O)— | methyl |
| | 0 | 1 | 2 | identical | (R¹ O)₂P(O)— | methyl |
| | 0 | 2 | 2 | identical | (R¹ O)₂P(O)— | methyl |
| | 1 | 1 | 0 | identical | (R¹ O)₂P(O)— | methyl |
| | 1 | 2 | 0 | identical | (R¹ O)₂P(O)— | methyl |
| | 1 | 1 | 1 | identical | (R¹ O)₂P(O)— | methyl |
| | 1 | 2 | 1 | identical | (R¹ O)₂P(O)— | methyl |
| | 1 | 1 | 2 | identical | (R¹ O)₂P(O)— | methyl |
| | 1 | 2 | 2 | identical | (R¹ O)₂P(O)— | methyl |
| | 0 | 1 | 0 | different | (R¹ O)₂P(O)— | methyl |
| | 0 | 2 | 0 | different | (R¹ O)₂P(O)— | methyl |
| | 0 | 1 | 1 | different | (R¹ O)₂P(O)— | methyl |
| | 0 | 2 | 1 | different | (R¹ O)₂P(O)— | methyl |
| | 0 | 1 | 2 | different | (R¹ O)₂P(O)— | methyl |
| | 0 | 2 | 2 | different | (R¹ O)₂P(O)— | methyl |
| | 1 | 1 | 0 | different | (R¹ O)₂P(O)— | methyl |
| | 1 | 2 | 0 | different | (R¹ O)₂P(O)— | methyl |
| | 1 | 1 | 1 | different | (R¹ O)₂P(O)— | methyl |
| | 1 | 2 | 1 | different | (R¹ O)₂P(O)— | methyl |
| | 1 | 1 | 2 | different | (R¹ O)₂P(O)— | methyl |
| | 1 | 2 | 2 | different | (R¹ O)₂P(O)— | methyl |

or

| No. | m | n | o | Sp1/Sp2 | Z | R¹ |
|---|---|---|---|---|---|---|
| | 0 | 1 | 0 | identical | (R¹ O)₂P(O)— | ethyl |
| | 0 | 2 | 0 | identical | (R¹ O)₂P(O)— | ethyl |
| | 0 | 1 | 1 | identical | (R¹ O)₂P(O)— | ethyl |
| | 0 | 2 | 1 | identical | (R¹ O)₂P(O)— | ethyl |
| | 0 | 1 | 2 | identical | (R¹ O)₂P(O)— | ethyl |
| | 0 | 2 | 2 | identical | (R¹ O)₂P(O)— | ethyl |
| | 1 | 1 | 0 | identical | (R¹ O)₂P(O)— | ethyl |
| | 1 | 2 | 0 | identical | (R¹ O)₂P(O)— | ethyl |
| | 1 | 1 | 1 | identical | (R¹ O)₂P(O)— | ethyl |
| | 1 | 2 | 1 | identical | (R¹ O)₂P(O)— | ethyl |
| | 1 | 1 | 2 | identical | (R¹ O)₂P(O)— | ethyl |
| | 1 | 2 | 2 | identical | (R¹ O)₂P(O)— | ethyl |
| | 0 | 1 | 0 | different | (R¹ O)₂P(O)— | ethyl |
| | 0 | 2 | 0 | different | (R¹ O)₂P(O)— | ethyl |
| | 0 | 1 | 1 | different | (R¹ O)₂P(O)— | ethyl |
| | 0 | 2 | 1 | different | (R¹ O)₂P(O)— | ethyl |
| | 0 | 1 | 2 | different | (R¹ O)₂P(O)— | ethyl |
| | 0 | 2 | 2 | different | (R¹ O)₂P(O)— | ethyl |
| | 1 | 1 | 0 | different | (R¹ O)₂P(O)— | ethyl |
| | 1 | 2 | 0 | different | (R¹ O)₂P(O)— | ethyl |
| | 1 | 1 | 1 | different | (R¹ O)₂P(O)— | ethyl |
| | 1 | 2 | 1 | different | (R¹ O)₂P(O)— | ethyl |
| | 1 | 1 | 2 | different | (R¹ O)₂P(O)— | ethyl |
| | 1 | 2 | 2 | different | (R¹ O)₂P(O)— | ethyl |

Further particular examples of parameter combinations of Z, m, n and o as well as SP¹ and SP² are:

| No. | m | n | o | Sp1/Sp2 | Z |
|---|---|---|---|---|---|
| | 0 | 1 | 0 | identical | —(O—(CH₂)ₓ)_{y}—OR⁶ |
| | 0 | 2 | 0 | identical | —(O—(CH₂)ₓ)_{y}—OR⁶ |
| | 0 | 1 | 1 | identical | —(O—(CH₂)ₓ)_{y}—OR⁶ |
| | 0 | 2 | 1 | identical | —(O—(CH₂)ₓ)_{y}—OR⁶ |
| | 0 | 1 | 2 | identical | —(O—(CH₂)ₓ)_{y}—OR⁶ |
| | 0 | 2 | 2 | identical | —(O—(CH₂)ₓ)_{y}—OR⁶ |
| | 1 | 1 | 0 | identical | —(O—(CH₂)ₓ)_{y}—OR⁶ |
| | 1 | 2 | 0 | identical | —(O—(CH₂)ₓ)_{y}—OR⁶ |
| | 1 | 1 | 1 | identical | —(O—(CH₂)ₓ)_{y}—OR⁶ |
| | 1 | 2 | 1 | identical | —(O—(CH₂)ₓ)_{y}—OR⁶ |
| | 1 | 1 | 2 | identical | —(O—(CH₂)ₓ)_{y}—OR⁶ |
| | 1 | 2 | 2 | identical | —(O—(CH₂)ₓ)_{y}—OR⁶ |
| | 0 | 1 | 0 | different | —(O—(CH₂)ₓ)_{y}—OR⁶ |
| | 0 | 2 | 0 | different | —(O—(CH₂)ₓ)_{y}—OR⁶ |
| | 0 | 1 | 1 | different | —(O—(CH₂)ₓ)_{y}—OR⁶ |
| | 0 | 2 | 1 | different | —(O—(CH₂)ₓ)_{y}—OR⁶ |
| | 0 | 1 | 2 | different | —(O—(CH₂)ₓ)_{y}—OR⁶ |
| | 0 | 2 | 2 | different | —(O—(CH₂)ₓ)_{y}—OR⁶ |
| | 1 | 1 | 0 | different | —(O—(CH₂)ₓ)_{y}—OR⁶ |
| | 1 | 2 | 0 | different | —(O—(CH₂)ₓ)_{y}—OR⁶ |
| | 1 | 1 | 1 | different | —(O—(CH₂)ₓ)_{y}—OR⁶ |
| | 1 | 2 | 1 | different | —(O—(CH₂)ₓ)_{y}—OR⁶ |
| | 1 | 1 | 2 | different | —(O—(CH₂)ₓ)_{y}—OR⁶ |
| | 1 | 2 | 2 | different | —(O—(CH₂)ₓ)_{y}—OR⁶ |

wherein
residue R⁶ represents H or lower alkyl, in particular H, methyl or ethyl;
x represents an integer selected from 1 or 2; and
y represents an integer from 1 to 20, in particular 1 to 15, 1 to 10 or 1 to 4; as for example 1, 2, 3, or 4,

Particular examples of parameter combinations of Z, x, y, R⁶, m, n and o as well as SP¹ and SP² are:

| No. | m | n | o | Sp1/Sp2 | Z | x | y | R⁶ |
|---|---|---|---|---|---|---|---|---|
| | 0 | 1 | 0 | identical | —(O—(CH₂)ₓ)_{y}—OR⁶ | 1 | 1-20 | H |
| | 0 | 2 | 0 | identical | —(O—(CH₂)ₓ)_{y}—OR⁶ | 1 | 1-20 | H |
| | 0 | 1 | 1 | identical | —(O—(CH₂)ₓ)_{y}—OR⁶ | 1 | 1-20 | H |
| | 0 | 2 | 1 | identical | —(O—(CH₂)ₓ)_{y}—OR⁶ | 1 | 1-20 | H |
| | 0 | 1 | 2 | identical | —(O—(CH₂)ₓ)_{y}—OR⁶ | 1 | 1-20 | H |
| | 0 | 2 | 2 | identical | —(O—(CH₂)ₓ)_{y}—OR⁶ | 1 | 1-20 | H |
| | 1 | 1 | 0 | identical | —(O—(CH₂)ₓ)_{y}—OR⁶ | 1 | 1-20 | H |
| | 1 | 2 | 0 | identical | —(O—(CH₂)ₓ)_{y}—OR⁶ | 1 | 1-20 | H |
| | 1 | 1 | 1 | identical | —(O—(CH₂)ₓ)_{y}—OR⁶ | 1 | 1-20 | H |
| | 1 | 2 | 1 | identical | —(O—(CH₂)ₓ)_{y}—OR⁶ | 1 | 1-20 | H |
| | 1 | 1 | 2 | identical | —(O—(CH₂)ₓ)_{y}—OR⁶ | 1 | 1-20 | H |
| | 1 | 2 | 2 | identical | —(O—(CH₂)ₓ)_{y}—OR⁶ | 1 | 1-20 | H |
| | 0 | 1 | 0 | different | —(O—(CH₂)ₓ)_{y}—OR⁶ | 1 | 1-20 | H |
| | 0 | 2 | 0 | different | —(O—(CH₂)ₓ)_{y}—OR⁶ | 1 | 1-20 | H |
| | 0 | 1 | 1 | different | —(O—(CH₂)ₓ)_{y}—OR⁶ | 1 | 1-20 | H |
| | 0 | 2 | 1 | different | —(O—(CH₂)ₓ)_{y}—OR⁶ | 1 | 1-20 | H |
| | 0 | 1 | 2 | different | —(O—(CH₂)ₓ)_{y}—OR⁶ | 1 | 1-20 | H |
| | 0 | 2 | 2 | different | —(O—(CH₂)ₓ)_{y}—OR⁶ | 1 | 1-20 | H |
| | 1 | 1 | 0 | different | —(O—(CH₂)ₓ)_{y}—OR⁶ | 1 | 1-20 | H |
| | 1 | 2 | 0 | different | —(O—(CH₂)ₓ)_{y}—OR⁶ | 1 | 1-20 | H |
| | 1 | 1 | 1 | different | —(O—(CH₂)ₓ)_{y}—OR⁶ | 1 | 1-20 | H |
| | 1 | 2 | 1 | different | —(O—(CH₂)ₓ)_{y}—OR⁶ | 1 | 1-20 | H |
| | 1 | 1 | 2 | different | —(O—(CH₂)ₓ)_{y}—OR⁶ | 1 | 1-20 | H |
| | 1 | 2 | 2 | different | —(O—(CH₂)ₓ)_{y}—OR⁶ | 1 | 1-20 | H |

or

| No. | m | n | o | Sp1/Sp2 | Z | x | y | R⁶ |
|---|---|---|---|---|---|---|---|---|
| | 0 | 1 | 0 | identical | —(O—(CH₂)ₓ)_{y}—OR⁶ | 2 | 1-20 | H |
| | 0 | 2 | 0 | identical | —(O—(CH₂)ₓ)_{y}—OR⁶ | 2 | 1-20 | H |
| | 0 | 1 | 1 | identical | —(O—(CH₂)ₓ)_{y}—OR⁶ | 2 | 1-20 | H |
| | 0 | 2 | 1 | identical | —(O—(CH₂)ₓ)_{y}—OR⁶ | 2 | 1-20 | H |
| | 0 | 1 | 2 | identical | —(O—(CH₂)ₓ)_{y}—OR⁶ | 2 | 1-20 | H |
| | 0 | 2 | 2 | identical | —(O—(CH₂)ₓ)_{y}—OR⁶ | 2 | 1-20 | H |
| | 1 | 1 | 0 | identical | —(O—(CH₂)ₓ)_{y}—OR⁶ | 2 | 1-20 | H |
| | 1 | 2 | 0 | identical | —(O—(CH₂)ₓ)_{y}—OR⁶ | 2 | 1-20 | H |
| | 1 | 1 | 1 | identical | —(O—(CH₂)ₓ)_{y}—OR⁶ | 2 | 1-20 | H |
| | 1 | 2 | 1 | identical | —(O—(CH₂)ₓ)_{y}—OR⁶ | 2 | 1-20 | H |
| | 1 | 1 | 2 | identical | —(O—(CH₂)ₓ)_{y}—OR⁶ | 2 | 1-20 | H |
| | 1 | 2 | 2 | identical | —(O—(CH₂)ₓ)_{y}—OR⁶ | 2 | 1-20 | H |
| | 0 | 1 | 0 | different | —(O—(CH₂)ₓ)_{y}—OR⁶ | 2 | 1-20 | H |
| | 0 | 2 | 0 | different | —(O—(CH₂)ₓ)_{y}—OR⁶ | 2 | 1-20 | H |
| | 0 | 1 | 1 | different | —(O—(CH₂)ₓ)_{y}—OR⁶ | 2 | 1-20 | H |
| | 0 | 2 | 1 | different | —(O—(CH₂)ₓ)_{y}—OR⁶ | 2 | 1-20 | H |
| | 0 | 1 | 2 | different | —(O—(CH₂)ₓ)_{y}—OR⁶ | 2 | 1-20 | H |
| | 0 | 2 | 2 | different | —(O—(CH₂)ₓ)_{y}—OR⁶ | 2 | 1-20 | H |
| | 1 | 1 | 0 | different | —(O—(CH₂)ₓ)_{y}—OR⁶ | 2 | 1-20 | H |
| | 1 | 2 | 0 | different | —(O—(CH₂)ₓ)_{y}—OR⁶ | 2 | 1-20 | H |
| | 1 | 1 | 1 | different | —(O—(CH₂)ₓ)_{y}—OR⁶ | 2 | 1-20 | H |
| | 1 | 2 | 1 | different | —(O—(CH₂)ₓ)_{y}—OR⁶ | 2 | 1-20 | H |
| | 1 | 1 | 2 | different | —(O—(CH₂)ₓ)_{y}—OR⁶ | 2 | 1-20 | H |
| | 1 | 2 | 2 | different | —(O—(CH₂)ₓ)_{y}—OR⁶ | 2 | 1-20 | H |

or

| No. | m | n | o | Sp1/Sp2 | Z | x | y | R⁶ |
|---|---|---|---|---|---|---|---|---|
| | 0 | 1 | 0 | identical | —(O—(CH₂)ₓ)_{y}—OR⁶ | 1 | 1-20 | methyl |
| | 0 | 2 | 0 | identical | —(O—(CH₂)ₓ)_{y}—OR⁶ | 1 | 1-20 | methyl |
| | 0 | 1 | 1 | identical | —(O—(CH₂)ₓ)_{y}—OR⁶ | 1 | 1-20 | methyl |
| | 0 | 2 | 1 | identical | —(O—(CH₂)ₓ)_{y}—OR⁶ | 1 | 1-20 | methyl |
| | 0 | 1 | 2 | identical | —(O—(CH₂)ₓ)_{y}—OR⁶ | 1 | 1-20 | methyl |
| | 0 | 2 | 2 | identical | —(O—(CH₂)ₓ)_{y}—OR⁶ | 1 | 1-20 | methyl |
| | 1 | 1 | 0 | identical | —(O—(CH₂)ₓ)_{y}—OR⁶ | 1 | 1-20 | methyl |
| | 1 | 2 | 0 | identical | —(O—(CH₂)ₓ)_{y}—OR⁶ | 1 | 1-20 | methyl |
| | 1 | 1 | 1 | identical | —(O—(CH₂)ₓ)_{y}—OR⁶ | 1 | 1-20 | methyl |
| | 1 | 2 | 1 | identical | —(O—(CH₂)ₓ)_{y}—OR⁶ | 1 | 1-20 | methyl |
| | 1 | 1 | 2 | identical | —(O—(CH₂)ₓ)_{y}—OR⁶ | 1 | 1-20 | methyl |
| | 1 | 2 | 2 | identical | —(O—(CH₂)ₓ)_{y}—OR⁶ | 1 | 1-20 | methyl |
| | 0 | 1 | 0 | different | —(O—(CH₂)ₓ)_{y}—OR⁶ | 1 | 1-20 | methyl |
| | 0 | 2 | 0 | different | —(O—(CH₂)ₓ)_{y}—OR⁶ | 1 | 1-20 | methyl |
| | 0 | 1 | 1 | different | —(O—(CH₂)ₓ)_{y}—OR⁶ | 1 | 1-20 | methyl |
| | 0 | 2 | 1 | different | —(O—(CH₂)ₓ)_{y}—OR⁶ | 1 | 1-20 | methyl |
| | 0 | 1 | 2 | different | —(O—(CH₂)ₓ)_{y}—OR⁶ | 1 | 1-20 | methyl |
| | 0 | 2 | 2 | different | —(O—(CH₂)ₓ)_{y}—OR⁶ | 1 | 1-20 | methyl |
| | 1 | 1 | 0 | different | —(O—(CH₂)ₓ)_{y}—OR⁶ | 1 | 1-20 | methyl |
| | 1 | 2 | 0 | different | —(O—(CH₂)ₓ)_{y}—OR⁶ | 1 | 1-20 | methyl |
| | 1 | 1 | 1 | different | —(O—(CH₂)ₓ)_{y}—OR⁶ | 1 | 1-20 | methyl |
| | 1 | 2 | 1 | different | —(O—(CH₂)ₓ)_{y}—OR⁶ | 1 | 1-20 | methyl |
| | 1 | 1 | 2 | different | —(O—(CH₂)ₓ)_{y}—OR⁶ | 1 | 1-20 | methyl |
| | 1 | 2 | 2 | different | —(O—(CH₂)ₓ)_{y}—OR⁶ | 1 | 1-20 | methyl |

or

| No. | m | n | o | Sp1/Sp2 | Z | x | y | R⁶ |
|---|---|---|---|---|---|---|---|---|
| | 0 | 1 | 0 | identical | —(O—(CH₂)ₓ)_{y}—OR⁶ | 2 | 1-20 | ethyl |
| | 0 | 2 | 0 | identical | —(O—(CH₂)ₓ)_{y}—OR⁶ | 2 | 1-20 | ethyl |
| | 0 | 1 | 1 | identical | —(O—(CH₂)ₓ)_{y}—OR⁶ | 2 | 1-20 | ethyl |
| | 0 | 2 | 1 | identical | —(O—(CH₂)ₓ)_{y}—OR⁶ | 2 | 1-20 | ethyl |
| | 0 | 1 | 2 | identical | —(O—(CH₂)ₓ)_{y}—OR⁶ | 2 | 1-20 | ethyl |
| | 0 | 2 | 2 | identical | —(O—(CH₂)ₓ)_{y}—OR⁶ | 2 | 1-20 | ethyl |
| | 1 | 1 | 0 | identical | —(O—(CH₂)ₓ)_{y}—OR⁶ | 2 | 1-20 | ethyl |
| | 1 | 2 | 0 | identical | —(O—(CH₂)ₓ)_{y}—OR⁶ | 2 | 1-20 | ethyl |
| | 1 | 1 | 1 | identical | —(O—(CH₂)ₓ)_{y}—OR⁶ | 2 | 1-20 | ethyl |
| | 1 | 2 | 1 | identical | —(O—(CH₂)ₓ)_{y}—OR⁶ | 2 | 1-20 | ethyl |
| | 1 | 1 | 2 | identical | —(O—(CH₂)ₓ)_{y}—OR⁶ | 2 | 1-20 | ethyl |
| | 1 | 2 | 2 | identical | —(O—(CH₂)ₓ)_{y}—OR⁶ | 2 | 1-20 | ethyl |
| | 0 | 1 | 0 | different | —(O—(CH₂)ₓ)_{y}—OR⁶ | 2 | 1-20 | ethyl |
| | 0 | 2 | 0 | different | —(O—(CH₂)ₓ)_{y}—OR⁶ | 2 | 1-20 | ethyl |
| | 0 | 1 | 1 | different | —(O—(CH₂)ₓ)_{y}—OR⁶ | 2 | 1-20 | ethyl |
| | 0 | 2 | 1 | different | —(O—(CH₂)ₓ)_{y}—OR⁶ | 2 | 1-20 | ethyl |
| | 0 | 1 | 2 | different | —(O—(CH₂)ₓ)_{y}—OR⁶ | 2 | 1-20 | ethyl |
| | 0 | 2 | 2 | different | —(O—(CH₂)ₓ)_{y}—OR⁶ | 2 | 1-20 | ethyl |
| | 1 | 1 | 0 | different | —(O—(CH₂)ₓ)_{y}—OR⁶ | 2 | 1-20 | ethyl |
| | 1 | 2 | 0 | different | —(O—(CH₂)ₓ)_{y}—OR⁶ | 2 | 1-20 | ethyl |
| | 1 | 1 | 1 | different | —(O—(CH₂)ₓ)_{y}—OR⁶ | 2 | 1-20 | ethyl |
| | 1 | 2 | 1 | different | —(O—(CH₂)ₓ)_{y}—OR⁶ | 2 | 1-20 | ethyl |
| | 1 | 1 | 2 | different | —(O—(CH₂)ₓ)_{y}—OR⁶ | 2 | 1-20 | ethyl |
| | 1 | 2 | 2 | different | —(O—(CH₂)ₓ)_{y}—OR⁶ | 2 | 1-20 | ethyl |

According to a third embodiment of the present invention the spacer Sₚ¹ of the compound of formula I of anyone of the preceding embodiments is absent or, more particularly, selected from
a) mono- or polycyclic optionally mono- or poly-substituted aromatic moieties having 6 to 14 ring carbon atoms, in particular 1,4-phenylene, wherein said one or more optional substituents are independently of each other selected from -Hal, —CHal₃, —OH, —SH, —NR′₂, —NO₂, —CN, —C(═O)R″, —C(═O)OR′″, alkyl, alkenyl, alkynyl, and alkoxy;
wherein
R′, R″ and R′″ independently of each other are selected from H and C₁- to C₄-alkyl; (Moiety M1);
b) heterocyclic residues of the general formula X

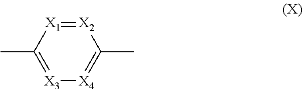

wherein
one, two or three of the ring moieties X₁ to X₄ represents N and the other represent >CH; (Moiety M2);
c) linear or branched lower-alkylene, in particular —(CH₂)ₙ₁—, wherein n1 is an integer from 1 to 4; more particularly methylene; (Moiety M3); and
d) combinations of at least two identical or, more particularly, different moieties, selected from M1, M2 and M3.

### As Regards Moiety M1:

According to a particular embodiment Moiety M1 is a monocyclic non-substituted aromatic moiety having 6 carbon atoms, in particular 1,4-phenylene, or

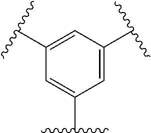 [SMILES: *c1cc(*)cc(*)c1]

### As Regards Moiety M2:

According to a particular embodiment Moiety M2 represents heterocyclic residues of the general formula X, wherein

One or two of the ring moieties X₁ to X₄ represents N and the other represent >CH;

### As Regards Moiety M3:

According to a particular embodiment Moiety M3 represents a linear lower-alkylene, in particular —(CH₂)ₙ₁—, wherein n1 is an integer from 1 or 2; more particularly methylene;

According to a fourth embodiment of the present invention the spacer Sₚ² of a compound (I) of anyone of the preceding embodiments is selected from
a) mono- or polycyclic optionally mono- or poly-substituted aromatic moieties having 6 to 14 ring carbon atoms, in particular 1,2-phenylene 1,3-phenylene or 1,4-phenylene; wherein said one or more optional substituents are independently of each other selected from -Hal, —CHal₃, —OH, —SH, —NR′₂, NO₂, —CN, —C(═O)R″, —C(═O)OR′″, alkyl, alkenyl, alkynyl, and alkoxy;
   wherein
   R′, R″ and R′″ independently of each other are selected from H and C₁- to C₄-alkyl; (Moiety M1);
b) heterocyclic residues of the general formula X

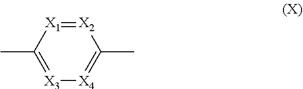

wherein
one, two or three of the ring moieties X₁ to X₄ represents N and the other represent >CH; (Moiety M2);
c) linear or branched lower-alkylene, in particular —(CH₂)ₙ₁—,
wherein
n1 is an integer from 1 to 4; more particularly methylene; (Moiety M3);
d) linear or branched polyalkylene oxide moieties, in particular selected from linear the moieties —((CH₂)ₓ₁—O)_{y1}— or —(O—(CH₂)ₓ₁)_{y1}— and the branched analogues thereof;
wherein
x1 independently of each other represent an integer selected from 1, 2, 3 or 4; in particular 1 or 2; and
y1 independently of each other represent an integer from 1 to 20, in particular 1 to 4; (Moiety M4);
e) a heteroatom containing moiety selected from
—N(R″″)—,
—(CH₂)ₓ₂—N(R″″)—;
—N(R″″)—(CH₂)ₓ₃—C(O)O—;
—N(R″″)—(CH₂)ₓ₃—C(O)—;
—N(R″″)—C(O)O—(CH₂)ₓ₄—N(R″″)—;
—N(R″″)—C(O)—(CH₂)ₓ₄—N(R″″)—;
—(CH₂)ₓ₄—C(O)—; and
—(CH₂)ₓ₄—C(O)O—
   wherein
   R″″ are independently of each other selected from H and C₁-C₄-alkyl
   x2 represents an integer selected from 1, 2, 3 or 4; in particular 1 or 2;
   x3 represents an integer selected from 1, 2, 3 or 4; in particular 1 or 2; and
   x4 represents an integer selected from 1, 2, 3 or 4; in particular 1 or 2;
   (Moiety M5); or
f) combinations of at least two identical or, more particularly, different moieties selected from M1, M2, M3 and M4; or combinations of at least two identical or, more particularly, different moieties selected from M1, M2, M3, M4 and M5.

### As Regards Moiety M1:

According to a particular embodiment Moiety M1 is a monocyclic non-substituted aromatic moiety having 6 carbon atoms, particularly 1,2-phenylene 1,3-phenylene or 1,4 phenylene, more particularly 1,4-phenylene, or

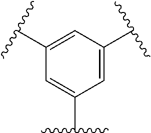 [SMILES: *c1cc(*)cc(*)c1]

### As Regards Moiety M2:

According to a particular embodiment Moiety M2 represents heterocyclic residues of the general formula X, wherein

One or two of the ring moieties X₁ to X₄ represents N and the other represent >CH;

### As Regards Moiety M3:

According to a particular embodiment Moiety M3 represents a linear lower-alkylene, in particular —(CH₂)ₙ₁—, wherein n1 is an integer from 1 or 2; more particularly methylene;

### As Regards Moiety M4:

According to a particular embodiment Moiety 4 represents a linear polyalkylene oxide moiety, selected from linear the moieties —((CH₂)ₓ₁—O)_{y1}— or —(O—(CH₂)ₓ₁)_{y1}—;
wherein
x1 independently of each other represent an integer selected from 1 or 2; and
y1 independently of each other represent an integer from 2 to 20, in particular 2 to 4;

### As Regards Moiety M5:

According to a particular embodiment, Moiety 5 represents —N(R″″)—, —N(R″″)—(CH₂)ₓ₃—C(O)— or —(CH₂)ₓ₂—N(R″″)—
wherein
R″″ are independently of each other selected from H and C₁-C₄-alkyl, more particularly H; and
x2 represents an integer selected from 1, 2, 3 or 4; in particular 1 or 2.

According to a fifth embodiment of the present invention said group A of the compound (I) of anyone of the preceding embodiments is an enzymatically or chemically cleavable linker group, selected from
a) a peptidyl group, in particular di-, tri- or tetra-peptidyl group;
b) a disulfide group of the formula —(CR⁷R⁸)ₙ₂—S—S—(CR⁷R⁸)ₙ₂—X₅— or X_{5′}—(CR⁷R⁶)ₙ₂—S—S—(CR⁷R⁸)ₙ₂—X₅—
   wherein
   n2 represents an integer from 1 to 4
   residues R⁷ and R⁸ independently of each other are selected from H or lower alkyl, in particular methyl; or two residues R⁷ and R⁸ together with the carbon atom which they are attached to form a cyclic C₄- to C₈-alkyl group; and
moiety X₅ is selected from —C(O)— and —O—;
moiety X_{5′} is selected from —C(O)— and —(O)C—(CH₂)—NH—;
c) hydrazone groups selected from >C═N—N(R⁹)— and —N(R⁹)—N═C<
   wherein
   R⁹ is H or lower alkyl; and
d) beta-glucuronidase-sensitive cleavable linker groups (glucuronide-linker groups), in particular carrying a beta-glucuronic acid derived trigger residue;

According to a particular embodiment thereof, the cleavable linker is a peptidyl group according to feature a).

According to another particular embodiment thereof, the cleavable linker is a glucuronide-linker group according to feature d).

According to a sixth embodiment of the present invention, said self-immolative group X of a compound (1) of anyone of the preceding embodiments, is selected from
a) p-amino-benzyl alcohol (PAB) derived groups of the formula
   —NH-p-phenylene-CH₂—O— or —O—CH₂-p-phenylene-NH— or
   —NH-p-phenylene-CH₂—N⁺(R²⁰)₂—
b) —O—C(O)—O—;
c) —O—C(O)—NR¹⁰—(CR¹²R¹³)_{z}—NR¹¹—C(O)—O— or
   —X¹—C(O)—NR¹⁰—(CR¹²R¹³)_{z}—NR¹¹—C(O)—X²—
   wherein
      z represents an integer selected from 1 to 6, in particular 1 to 4;
      R²⁰ independently of each other, represent H or a lower alkyl group
      R¹⁰ and R¹¹, independently of each other, represent H or lower alkyl group
      R¹² and R¹³, independently of each other, represent H, methyl or ethyl, in particular H or methyl, especially H; and
      X¹ and X² independently of each other represent O, S or NR¹⁰
d) methylene alkoxy carbamates (MAC) type linkages of the formula
   —OC(O)—NR¹³—C(R¹⁴R¹⁵)—(O)—
   —OC(O)—NR¹³—C(R¹⁴R¹⁵)—(S)—
   —OC(O)—NR¹³—C(R¹⁴R¹⁵)—(NR¹⁶)— or
   —OC(O)—NR¹³—C(R¹⁴R¹⁵)—(NR¹⁶—C(O)O)—
   wherein
      R¹³, R¹⁴, R¹⁵, and R¹⁶, independently of each other represent H or lower alkyl, in particular, C₁ to C₄-alkyl.

According to a particular embodiment thereof, the said self-immolative group X is a PAB derived group according to feature a).

According to a seventh embodiment of the present invention said payload residue Y of a compound of anyone of the preceding embodiments, is selected from bioactive compounds; labeling agents, such as in particular dyes, radiolabels, protein degraders, photosensitizers; and chelators.

According to an eighth embodiment of the present invention, said compound of anyone of the preceding embodiments, corresponds to a compound of general formula Ia

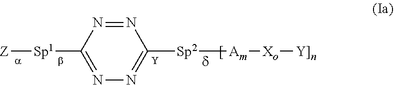

wherein linkages α, β, γ and δ are independently from each other selected from a chemical bond, or an ether, thioether, ester, amide, carbamate, carbonyl (in particular keto), dicarbamate, carbonate, hydrazine, urea, alkylene oxide or linear or branched polyalkylene oxide linkage.

Said polyalkylene oxide linkage, selected from linear the moieties —((CH₂)ₓ₁—O)_{y1}— or —(O—(CH₂)ₓ₁)_{y1}—;
wherein
x1 independently of each other represent an integer selected from 1 or 2; and
y1 independently of each other represent an integer from 2 to 20, in particular 2 to 4;

According to a particular embodiment linkages α, β, γ and δ each are a chemical bond.

According to another particular embodiment linkages α, β, γ and δ is a chemical bond.

According to another particular embodiment linkages β, is a chemical bond.

According to another particular embodiment linkages γ is a chemical bond.

According to another particular embodiment linkages α and β each are a chemical bond.

According to another particular embodiment linkages α, β and γ each are a chemical bond.

According to another particular embodiment linkages α, β, γ each are a chemical bond, and δ is an ether, thioether, ester, amide, carbamate, carbonyl (in particular keto), dicarbamate, carbonate, hydrazine, urea, alkylene oxide or linear or branched polyalkylene oxide linkage.

According to another particular embodiment linkages α, β, γ each are a chemical bond, and δ is an, ester, amide, carbamate, dicarbamate, carbonate, alkylene oxide or linear or branched polyalkylene oxide linkage.

Said polyalkylene oxide linkage, selected from linear the moieties —((CH₂)ₓ₁—O)_{y1}— or —(O—(CH₂)ₓ₁)_{y1}—;
wherein
x1 independently of each other represent an integer selected from 1 or 2; and
y1 independently of each other represent an integer from 2 to 4;

According to a ninth embodiment of the present invention said spacer Sp¹ of a compound of anyone of the preceding embodiments is selected from one of the following combinations of moieties:
-M1-M3-, -M2-M3-, -M3-M1- or M3-M2-
wherein
the linkages between said moieties M1, M2, M3 are independently selected from a chemical bond, an ether, thioether, ester, amide, carbamate, dicarbamate, carbonate, hydrazine or urea, and alkylene oxide or a linear or branched polyalkylene oxide linkage.

In a particular embodiment the linkages between said moieties M1, M2, M3 are each are a chemical bond.

According to a tenth embodiment of the present invention said spacer Sp² of a compound of anyone of the preceding embodiments is selected from one of the following combinations of moieties
-M1-M3-, -M1-M4-, -M2-M3-, -M2-M5-, -M2-M4-, -M3-M1-, -M3-M2-, -M3-M4-. -M1-M3-M4-, -M1-M4-M3-, -M2-M3-M4-, -M2-M4-M3-, -M3-M2-M4-, -M3-M4-M2- or -M2-M5-M4-
wherein
the linkages between said moieties M1, M2, M3, M4 and M5 are independently selected from a chemical bond, an ether, thioether, ester, amide, carbamate, dicarbamate, carbonate, hydrazine or urea, and alkylene oxide or a linear or branched polyalkylene oxide linkage.

In a particular embodiment the linkages between said moieties M1, M2, M3 and M4 are each are a chemical bond.

Particular examples of combinations of motifs M for Sp¹ and Sp² are mentioned in the following table (where applicable spacer moieties may be present in a compound of formula I in any orientation)

| Sp¹ | | | Sp² | | | | |
|---|---|---|---|---|---|---|---|
| M1 | M2 | M3 | M1 | M2 | M3 | M4 | M5 |
| | | —CH₂— | 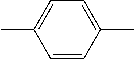 | | | | |
| | | —CH₂— | 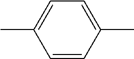 | | —CH₂— | | |
| 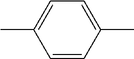 | | | | | —CH₂—CH₂— | | |
| | | —CH₂— | | | —CH₂—CH₂— | | |
| | | —CH₂— | | | —CH₂—CH₂— | | —NH— |
| | | —CH₂— | | | —CH₂— | | |
| | | —CH₂— | 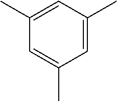 | | | | |
| | | —CH₂— | 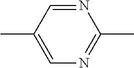 | | | | |
| | | —CH₂— | 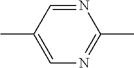 | | | | —NH— |
| | | —CH₂— | 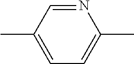 | | | | |
| | | —CH₂— | 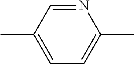 | | | | —NH—CH— |
| 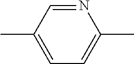 | | | 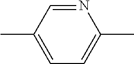 | | | | —NH— |
| | | —CH₂— | 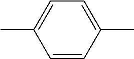 | | —CH₂— | —(OEt)₄—O— | |

Particular examples of compounds of Formula I with combinations of motifs M for Sp¹ and Sp² are mentioned in the following tables (where applicable spacer moieties may be present in a compound of formula I in any orientation):

| | Sp¹ | | | Sp² | | | | |
|---|---|---|---|---|---|---|---|---|
| Z | M1 | M2 | M3 | M1 | M2 | M3 | M4 | M5 |
| (R¹O)₂P(O)— | | | —CH₂— | 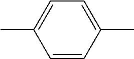 | | | | |
| (R¹O)₂P(O)— | | | —CH₂— | 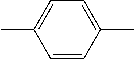 | | —CH₂— | | |
| (R¹O)₂P(O)— | 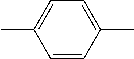 | | | | | —CH₂—CH₂— | | |
| (R¹O)₂P(O)— | | | —CH₂— | | | —CH₂—CH₂— | | |
| | | | —CH₂— | | | —CH₂—CH₂— | | —NH— |
| (R¹O)₂P(O)— | | | —CH₂— | | | —CH₂— | | |
| (R¹O)₂P(O)— | | | —CH₂— | 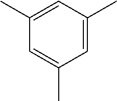 | | | | |
| (R¹O)₂P(O)— | | | —CH₂— | 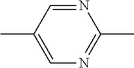 | | | | |
| (R¹O)₂P(O)— | | | —CH₂— | 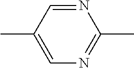 | | | | |
| (R¹O)₂P(O)— | | | —CH₂— | 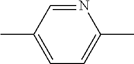 | | | | |
| (R¹O)₂P(O)— | | | —CH₂— | 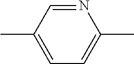 | | | | —NH—CH— |
| (R¹O)₂P(O)— | 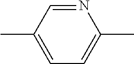 | | | 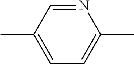 | | | | —NH— |
| (R¹O)₂P(O)— | | | —CH₂— | 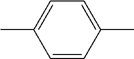 | | —CH₂— | —(OEt)₄—O— | |

wherein R¹ is H or lower alkyl
or

| | | Sp¹ | | | Sp² | | | | |
|---|---|---|---|---|---|---|---|---|---|
| Z | R¹ | M1 | M2 | M3 | M1 | M2 | M3 | M4 | M5 |
| (R¹O)₂P(O)— | H | | | —CH₂— | 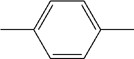 | | | | |
| (R¹O)₂P(O)— | H | | | —CH₂— | 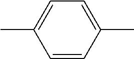 | | —CH₂— | | |
| (R¹O)₂P(O)— | H | 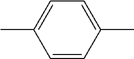 | | | | | —CH₂—CH₂— | | |
| (R¹O)₂P(O)— | H | | | —CH₂— | | | —CH₂—CH₂— | | |
| (R¹O)₂P(O)— | H | | | —CH₂— | | | —CH₂—CH₂— | | —NH— |
| (R¹O)₂P(O)— | H | | | —CH₂— | | | —CH₂— | | |
| (R¹O)₂P(O)— | H | | | —CH₂— | 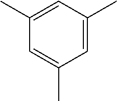 | | | | |
| (R¹O)₂P(O)— | H | | | —CH₂— | 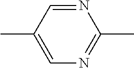 | | | | |
| (R¹O)₂P(O)— | H | | | —CH₂— | 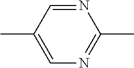 | | | | —NH— |
| (R¹O)₂P(O)— | H | | | —CH₂— | 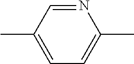 | | | | |
| (R¹O)₂P(O)— | H | | | —CH₂— | 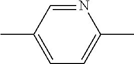 | | | | —NH—CH— |
| (R¹O)₂P(O)— | H | 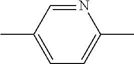 | | | 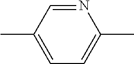 | | | | —NH— |
| (R¹O)₂P(O)— | H | | | —CH₂— | 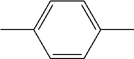 | | —CH₂— | —(OEt)₄—O— | |

or

| | | Sp¹ | | | Sp² | | | | |
|---|---|---|---|---|---|---|---|---|---|
| Z | R¹ | M1 | M2 | M3 | M1 | M2 | M3 | M4 | M5 |
| (R¹O)₂P(O)— | methyl | | | —CH₂— | 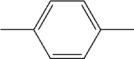 | | | | |
| (R¹O)₂P(O)— | methyl | | | —CH₂— | 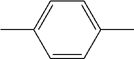 | | —CH₂— | | |
| (R¹O)₂P(O)— | methyl | 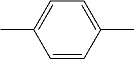 | | | | | —CH₂—CH₂— | | |
| (R¹O)₂P(O)— | methyl | | | —CH₂— | | | —CH₂—CH₂— | | |
| (R¹O)₂P(O)— | methyl | | | —CH₂— | | | —CH₂—CH₂— | | —NH— |
| (R¹O)₂P(O)— | methyl | | | —CH₂— | | | —CH₂— | | |
| (R¹O)₂P(O)— | methyl | | | —CH₂— | 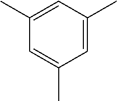 | | | | |
| (R¹O)₂P(O)— | methyl | | | —CH₂— | 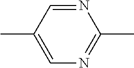 | | | | |
| (R¹O)₂P(O)— | methyl | | | —CH₂— | 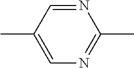 | | | | —NH— |
| (R¹O)₂P(O)— | methyl | | | —CH₂— | 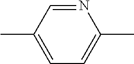 | | | | |
| (R¹O)₂P(O)— | methyl | | | —CH₂— | 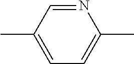 | | | | —NH—CH— |
| (R¹O)₂P(O)— | methyl | 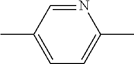 | | | 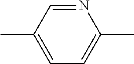 | | | | —NH— |
| (R¹O)₂P(O)— | methyl | | | —CH₂— | 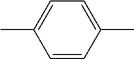 | | —CH₂— | —(OEt)₄—O— | |

or

| | | Sp¹ | | | Sp² | | | | |
|---|---|---|---|---|---|---|---|---|---|
| Z | R¹ | M1 | M2 | M3 | M1 | M2 | M3 | M4 | M5 |
| (R¹O)₂P(O)— | ethyl | | | —CH₂— | 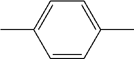 | | | | |
| (R¹O)₂P(O)— | ethyl | | | —CH₂— | 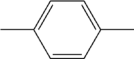 | | —CH₂— | | |
| (R¹O)₂P(O)— | ethyl | 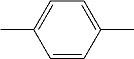 | | | | | —CH—CH₂— | | |
| (R¹O)₂P(O)— | ethyl | | | —CH₂— | | | —CH₂—CH₂— | | |
| (R¹O)₂P(O)— | ethyl | | | —CH₂— | | | —CH—CH₂— | | —NH— |
| (R¹O)₂P(O)— | ethyl | | | —CH₂— | | | —CH₂— | | |
| (R¹O)₂P(O)— | ethyl | | | —CH₂— | 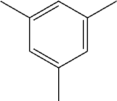 | | | | |
| (R¹O)₂P(O)— | ethyl | | | —CH₂— | 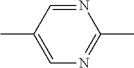 | | | | |
| (R¹O)₂P(O)— | ethyl | | | —CH₂— | 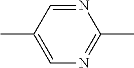 | | | | —NH— |
| (R¹O)₂P(O)— | ethyl | | | —CH₂— | 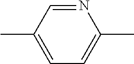 | | | | |
| (R¹O)₂P(O)— | ethyl | | | —CH₂— | 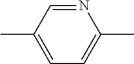 | | | — | —NH—CH— |
| (R¹O)₂P(O)— | ethyl | 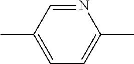 | | | 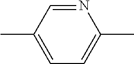 | | | | —NH— |
| (R¹O)₂P(O)— | ethyl | | | —CH₂— | 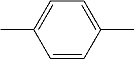 | | —CH₂— | —(OEt)₄—O— | |

or

| | Sp¹ | | | Sp² | | | |
|---|---|---|---|---|---|---|---|
| Z | M1 | M2 | M3 | M1 | M2 | M3 | M4 |
| —(O—(CH₂)ₓ)_{y}—OR⁶ | | | —CH₂— | 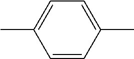 | | | |
| —(O—(CH₂)ₓ)_{y}—OR⁶ | | | —CH₂— | 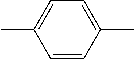 | | —CH₂— | |
| —(O—(CH₂)ₓ)_{y}—OR⁶ | | | —CH₂— | | | —CH₂— | |
| —(O—(CH₂)ₓ)_{y}—OR⁶ | | | —CH₂— | 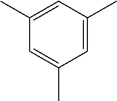 | | | |
| —(O—(CH₂)x)_{y}—OR⁶ | | | —CH₂— | 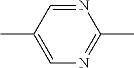 | | | |
| —(O—(CH₂)ₓ)_{y}—OR⁶ | | | —CH₂— | 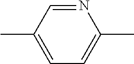 | | | |
| —(O—(CH₂)ₓ)_{y}—OR⁶ | | | —CH₂— | 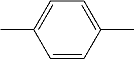 | | —CH₂— | —(OEt)₄—O— |

wherein
residue R⁶ represents H or lower alkyl, in particular H, methyl or ethyl;
x represents an integer selected from 1 or 2; and
y represents an integer from 1 to 20, in particular 1 to 15, 1 to 10 or 1 to 4; as for example 1, 2, 3, or 4,
or

| | | | | Sp¹ | | | Sp² | | | |
|---|---|---|---|---|---|---|---|---|---|---|
| Z | x | y | R⁶ | M1 | M2 | M3 | M1 | M2 | M3 | M4 |
| —(O—(CH₂)ₓ)_{y}—OR⁶ | 1 | 1-20 | H | | | —CH₂— | 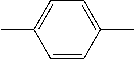 | | | |
| —(O—(CH₂)ₓ)_{y}—OR⁶ | 1 | 1-20 | H | | | —CH₂— | 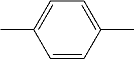 | | —CH₂— | |
| —(O—(CH₂)ₓ)_{y} —OR⁶ | 1 | 1-20 | H | | | —CH₂— | | | —CH₂— | |
| —(O—(CH₂)ₓ)_{y}—OR⁶ | 1 | 1-20 | H | | | —CH₂— | 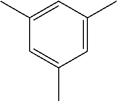 | | | |
| —(O—(CH₂)ₓ)_{y}—OR⁶ | 1 | 1-20 | H | | | —CH₂— | 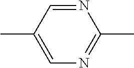 | | | |
| —(O—(CH₂)ₓ)_{y}—OR⁶ | 1 | 1-20 | H | | | —CH₂— | 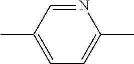 | | | |
| —(O—(CH₂)ₓ)_{y}—OR⁶ | 1 | 1-20 | H | | | —CH₂— | 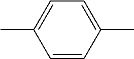 | | —CH₂— | —(OEt)₄—O— |

or

| | | | | Sp¹ | | | Sp² | | | |
|---|---|---|---|---|---|---|---|---|---|---|
| Z | x | y | R⁶ | M1 | M2 | M3 | M1 | M2 | M3 | M4 |
| —(O—(CH₂)ₓ)_{y}—OR⁶ | 2 | 1-20 | H | | | —CH₂— | 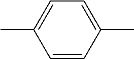 | | | |
| —(O—(CH₂)ₓ)_{y}—OR⁶ | 2 | 1-20 | H | | | —CH₂— | 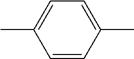 | | —CH₂— | |
| —(O—(CH₂)ₓ)_{y}—OR⁶ | 2 | 1-20 | H | | | —CH₂— | | | —CH₂— | |
| —(O—(CH₂)ₓ)_{y}—OR⁶ | 2 | 1-20 | H | | | —CH₂— | 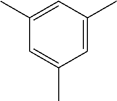 | | | |
| —(O—(CH₂)ₓ)_{y} —OR⁶ | 2 | 1-20 | H | | | —CH₂— | 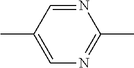 | | | |
| —(O—(CH₂)ₓ)_{y}—OR⁶ | 2 | 1-20 | H | | | —CH₂— | 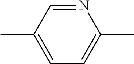 | | | |
| —(O—(CH₂)ₓ)_{y} —OR⁶ | 2 | 1-20 | H | | | —CH₂— | 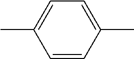 | | —CH₂— | —(OEt)₄—O— |
| —(O—(CH₂)ₓ)_{y}—OR⁶ | 1 | 1-20 | H | | | —CH₂— | 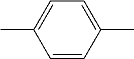 | | | |
| —(O—(CH₂)ₓ)_{y} —OR⁶ | 1 | 1-20 | H | | | —CH₂— | 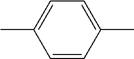 | | —CH₂— | |
| —(O—(CH₂)ₓ)_{y} —OR⁶ | 1 | 1-20 | H | | | —CH₂— | | | —CH₂— | |
| —(O—(CH₂)ₓ)_{y}—OR⁶ | 1 | 1-20 | H | | | —CH₂— | 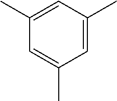 | | | |
| —(O—(CH₂)ₓ)_{y}—OR⁶ | 1 | 1-20 | H | | | —CH₂— | 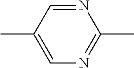 | | | |
| —(O—(CH₂)ₓ)_{y}—OR⁶ | 1 | 1-20 | H | | | —CH₂— | 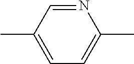 | | | |
| —(O—(CH₂)ₓ)_{y}—OR⁶ | 1 | 1-20 | H | | | —CH₂— | 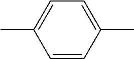 | | —CH₂— | —(OEt)₄—O— |

or

| | | | | Sp¹ | | | Sp² | | | |
|---|---|---|---|---|---|---|---|---|---|---|
| Z | x | y | R⁶ | M1 | M2 | M3 | M1 | M2 | M3 | M4 |
| —(O—(CH₂)ₓ)_{y}—OR⁶ | 1 | 1-20 | methyl | | | —CH₂— | 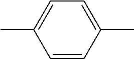 | | | |
| —(O—(CH₂)ₓ)_{y} —OR⁶ | 1 | 1-20 | methyl | | | —CH₂— | 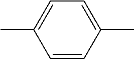 | | —CH₂— | |
| —(O—(CH₂)ₓ)_{y}—OR⁶ | 1 | 1-20 | methyl | | | —CH₂— | | | —CH₂— | |
| —(O—(CH₂)ₓ)_{y}—OR⁶ | 1 | 1-20 | methyl | | | —CH₂— | 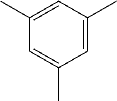 | | | |
| —(O—(CH₂)ₓ)_{y}—OR⁶ | 1 | 1-20 | methyl | | | —CH₂— | 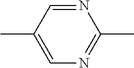 | | | |
| —(O—(CH₂)ₓ)_{y}—OR⁶ | 1 | 1-20 | methyl | | | —CH2— | 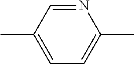 | | | |
| —(O—(CH₂)ₓ)_{y}—OR⁶ | 1 | 1-20 | Methyl | | | —CH₂— | 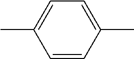 | | —CH₂— | —(OEt)₄—O— |

or

| | | | | Sp¹ | | | Sp² | | | |
|---|---|---|---|---|---|---|---|---|---|---|
| Z | x | y | R⁶ | M1 | M2 | M3 | M1 | M2 | M3 | M4 |
| —(O—(CH₂)ₓ)_{y}—OR⁶ | 2 | 1-20 | ethyl | | | —CH₂— | 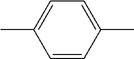 | | | |
| —(O—(CH₂)ₓ)_{y} —OR⁶ | 2 | 1-20 | ethyl | | | —CH₂— | 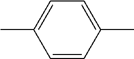 | | —CH₂— | |
| —(O—(CH₂)ₓ)_{y} —OR⁶ | 2 | 1-20 | ethyl | | | —CH₂— | | | —CH₂— | |
| —(O—(CH₂)ₓ)_{y}—OR⁶ | 2 | 1-20 | ethyl | | | —CH₂— | 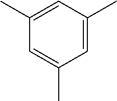 | | | |
| —(O—(CH₂)ₓ)_{y} —OR⁶ | 2 | 1-20 | ethyl | | | —CH₂— | 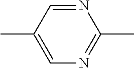 | | | |
| —(O—(CH₂)ₓ)_{y} —OR⁶ | 2 | 1-20 | ethyl | | | —CH₂— | 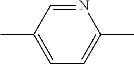 | | | |
| —(O−(CH₂)ₓ)_{y} —OR⁶ | 2 | 1-20 | ethyl | | | —CH₂— | 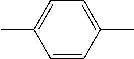 | | —CH₂— | —(OEt)₄—O— |

### 2. The Second Aspect of the Present Invention

The second aspect of the present invention relates to conjugates, and more particularly bio-conjugates. They are formed by the reaction of at least one functionalized payload molecule of the general formula (I) of the above first aspect of the invention, functionalized by means of a particular tetrazine group as defined above. Said payload molecule is adapted to conjugation with a functionalized targeting agent, in particular functionalized bio-molecule, carrying a functional counterpart group, which is reactive with said tetrazine group of the functionalized payload molecule in a biorthogonal chemical reaction.

According to an eleventh embodiment of the present invention a conjugate, and more particularly bio-conjugate, is provided, which is obtainable by reacting a functionalized targeting agent, with a tetrazine compound of formula I, of anyone of the preceding embodiments in order to form a covalent linkage between said functionalized targeting agent, and said tetrazine compound of formula I.

According to a twelfth embodiment of the present invention said functionalized targeting agent of the eleventh embodiment is selected from correspondingly functionalized forms of the following entities: viruses, whole cells, phages, liposomes, biomolecules and low-or-high-molecular weight chemical compounds, antibodies, antibody derivatives, antibody fragments, antibody (fragment) fusions, enzymes, proteins, peptides, peptide mimetics, carbohydrates, monosaccharides, polysaccharides, oligo- or polynucleotides, in particular DNA, RNA, PNA and LNA molecules, aptamers, drugs, glycoproteins, glycans, lipids, polymers, chemotherapeutic agents, receptor agonists and antagonists, cytokines, hormones, steroids, toxins and derivatives thereof. In a particular embodiment thereof, the targeting agent is selected from antibodies, antibody derivatives, antibody fragments, and antibody (fragment) fusions.

According to a thirteenth embodiment of the present invention a conjugate of anyone of the embodiments eleven and twelve is provided, wherein said functionalized targeting agent comprises as a functional group at least one dienophilic moiety reactive with said tetrazin moiety of said compound of formula I.

According to a fourteenth embodiment of the present invention conjugate of anyone of the embodiments eleven to thirteen is provided, wherein said functionalized targeting agent comprises at least one polypeptide sequence, having at least one non-natural amino acid residue within its amino acid sequence, which non-natural amino acid residue comprises at least one dienophile moiety reactive with said tetrazin moiety of said compound of formula I.

According to a fifteenth embodiment of the present invention a conjugate of anyone of the embodiments eleven to fourteen is provided, wherein said functionalized biomolecule is a polyclonal or monoclonal immunoglobulin molecule, in particular a monoclonal antibody or fragment thereof.

According to a sixteenth embodiment of the present invention a conjugate of anyone of the embodiments eleven to fifteen is provided, which is formed by biorthogonal bioconjugation of a tetrazine-compound of formula I and a functionalized biomolecule carrying a functional group capable of reaction via a Diels-Alder-type cycloaddition reaction, as for example cyclooctinyl-dienophiles, trans-cyclooctenyl-dienophiles, norbornenyl dienophiles, cyclopropenyl dienophiles, cyclobutenyl dienophiles, spirohexenyl dienophiles, BCN dienophiles, azetine dienophiles, or alkenes.

According to a seventeenth embodiment of the present invention a conjugate of embodiment sixteen is provided, wherein said functional group capable of reaction via a Diels-Alder-type cycloaddition reaction is selected from
(i) a trans-cyclooctenyl dienophile group of the formula:

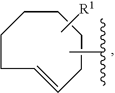

wherein
R¹ is hydrogen, halogen, C₁-C₄-alkyl, (R^{a}O)₂P(O)O—C₁-C₄-alkyl, (R^{b}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, Di-(C₁-C₄-alkyl)amino, C₂-C₅-alkenylamino, C₂-C₅-alkenyl-C₁-C₄-alkyl-amino or Di-(C₂-C₅-alkenyl)amino; and
R^{a}, R^{b} independently are hydrogen or C₂-C₅-alkanoyloxymethyl; or
(ii) a cyclooctynyl dienophile group of the formula:

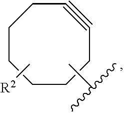

wherein
R² is hydrogen, halogen, C₁-C₄-alkyl, (R^{c}O)₂P(O)O—C₁-C₄-alkyl, (R^{d}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, Di-(C₁-C₄-alkyl)amino, C₂-C₅-alkenylamino, C₂-C₅-alkenyl-C₁-C₄-alkyl-amino or Di-(C₂-C₅-alkenyl)amino; and
R^{c}, R^{d} independently are hydrogen or C₂-C₅-alkanoyloxymethyl.

### 3. The Third Aspect of the Present Invention

A third aspect of the present invention relates to methods of preparing bio-conjugates.

According to an eighteenth embodiment of the present invention a method of preparing a bio-conjugate of anyone of the embodiments eleven to seventeen is provided. Said method comprises reaction in an aqueous, optionally buffered reaction medium a tetrazin compound as defined in anyone of the embodiments one to ten with a functionalized biomolecule carrying a functional dienophile group and performing a Diels-Alder-type cycloaddition reaction between said molecules.

### 4. The Fourth Aspect of the Present Invention

A fourth aspect of the present invention relates to certain tetrazine intermediates, for example useful for preparing the tetrazine compounds of formula I.

According to a nineteenth embodiment of the present invention a tetrazine intermediate of the general formula II is provided,

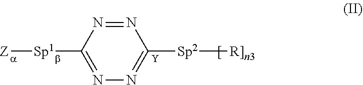

wherein
n3 represent an integer selected from 1 or 2
Sp¹ and Sp² are as defined above,
linkages α, β, and γ are independently from each other selected from a chemical bond, or an ether, thioether, ester, amide, carbonyl (in particular keto), carbamate, dicarbamate, carbonate, hydrazine, urea, alkylene oxide or linear or branched polyalkylene oxide linkage;
Z represents a phosphor containing hydrophilic group, in particular (R¹O)₂P(O)—, (R^{1a}O)₂P(O)—O—, and (R²O)₃P—O—;
   wherein
   R¹, R^{1a} and R² are same or different and independently of each other represent H or lower alkyl, in particular methyl or ethyl; and even more particularly H;
and
R represents H or a chemical group capable of forming a chemical bond, or capable of forming an ether, thioether, ester, such as active esters like succinimidyl- or pentafluorophenyl-ester, amide, carbamate, dicarbamate, carbonate, hydrazine, urea, alkylene oxide or linear or branched polyalkylene oxide linkage; and optionally with the proviso that R does not represent a chemical protecting group, in particular does not represents a cleavable protecting group, and more particularly not a N—, O—, or S-protecting group.

More particularly, R represents an amino or carboxyl group; According to a particular embodiment linkages α, β, and γ each are a chemical bond.

According to another particular embodiment linkages α is a chemical bond.

According to another particular embodiment linkages β, is a chemical bond.

According to another particular embodiment linkages γ is a chemical bond.

According to another particular embodiment linkages α and β each are a chemical bond.

According to another particular embodiment linkages α, β and γ each are a chemical bond.

According to another particular embodiment Z is (R¹O)₂P(O)—, (R^{1a}O)₂P(O)—O—, and (R²O)₃P—O—;
wherein
R¹, R^{1a} and R² are same or different and independently of each other represent H or lower alkyl, in particular methyl or ethyl; and even more particularly H;

In the following, particular preferred structures of compounds of general formula II are displayed. In said formulae residues R¹ independently of each other are as defined above.

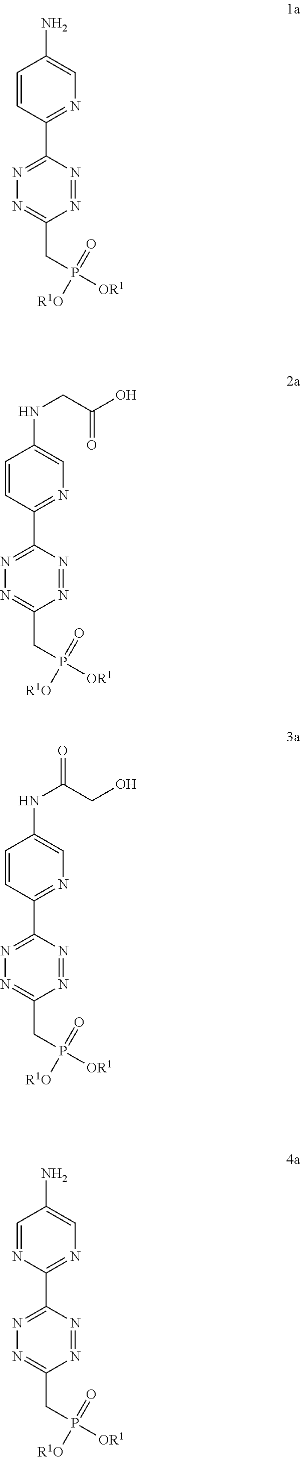

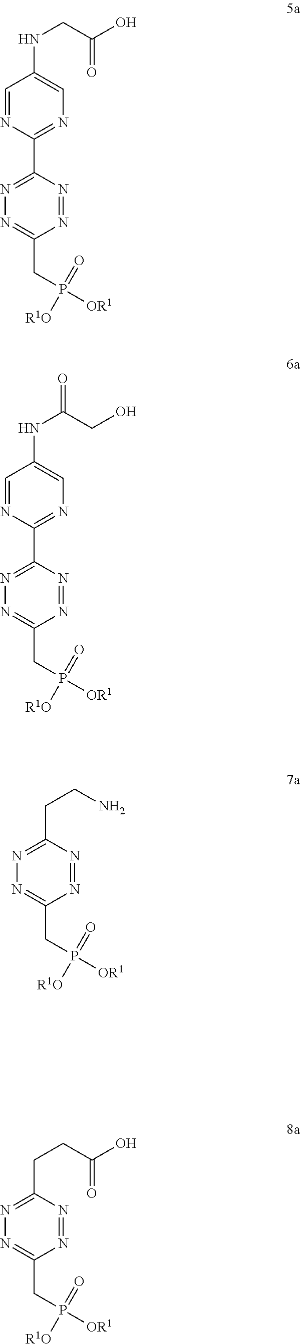

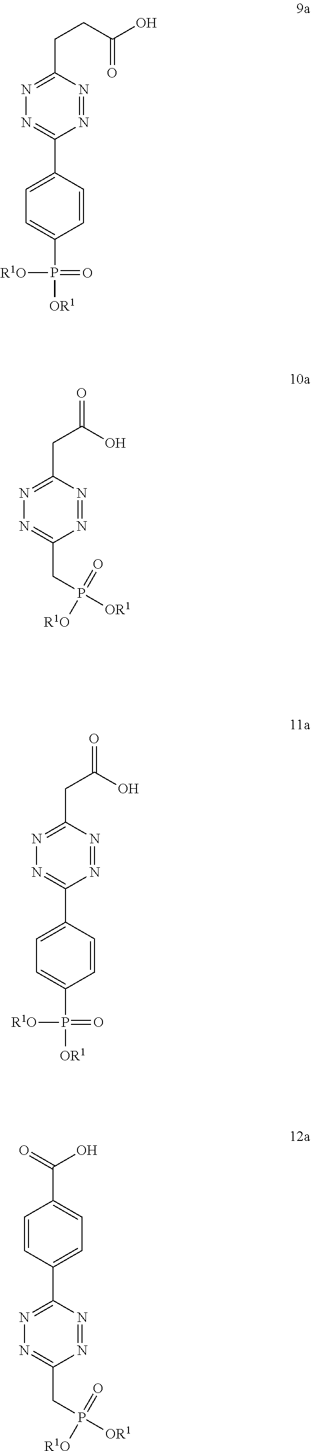

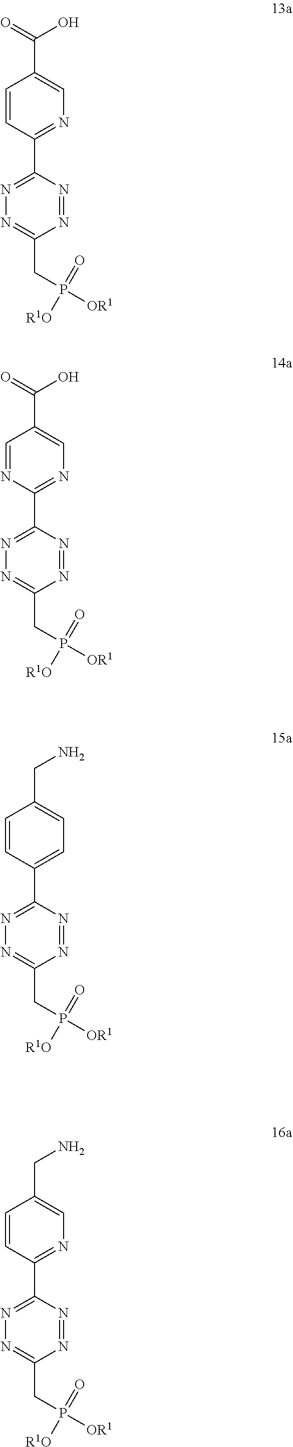

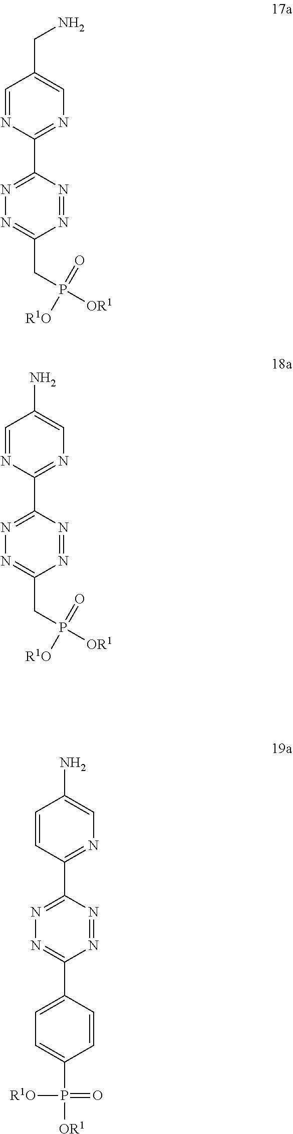

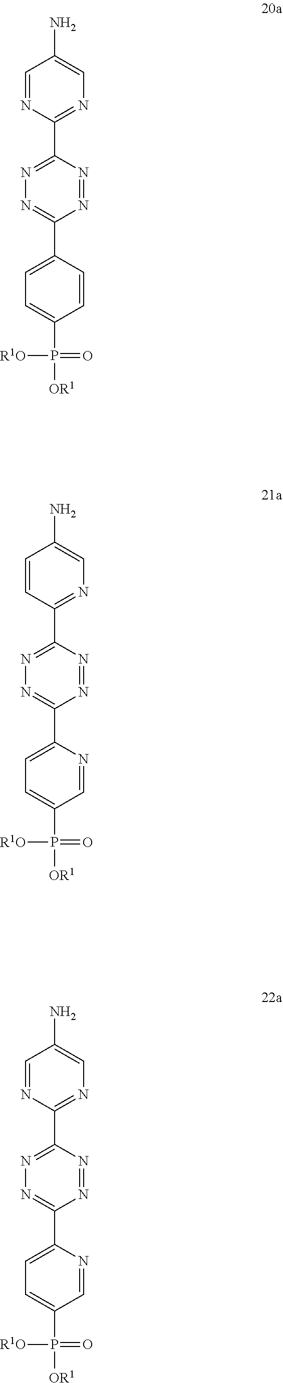

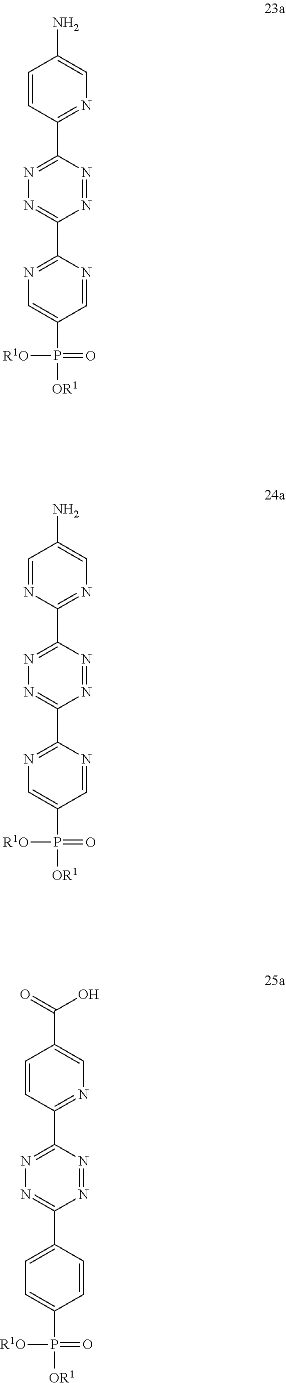

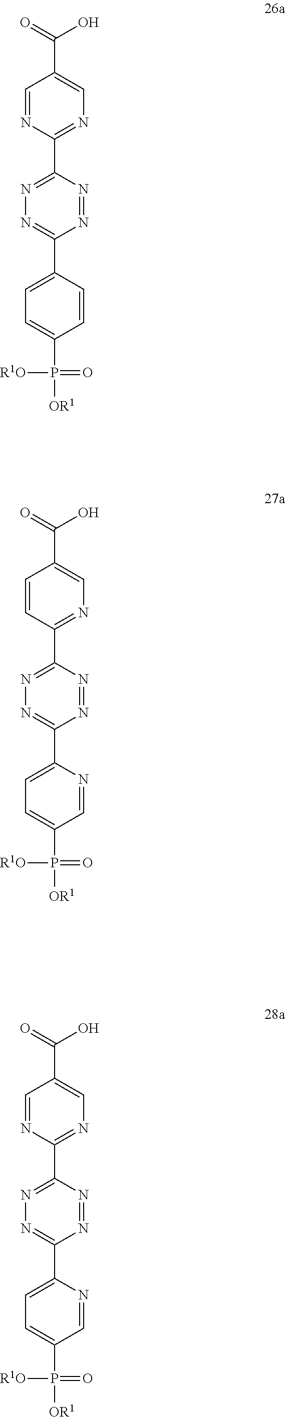

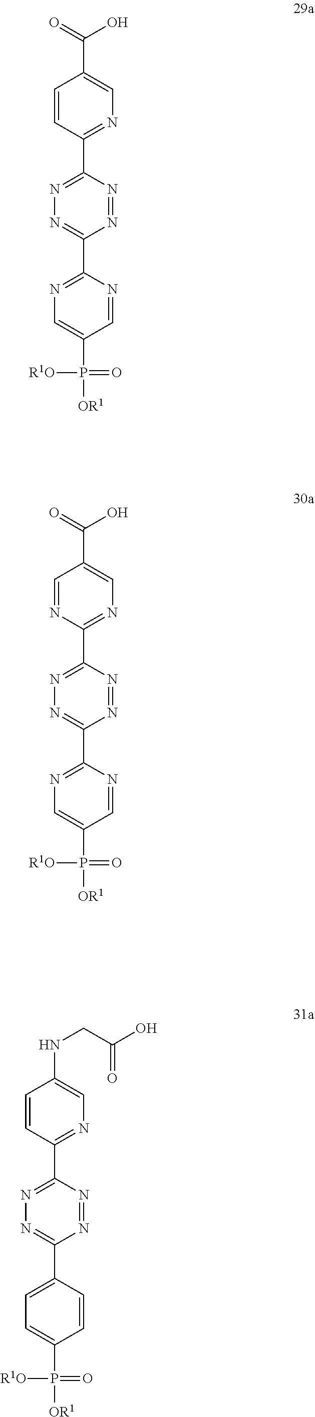

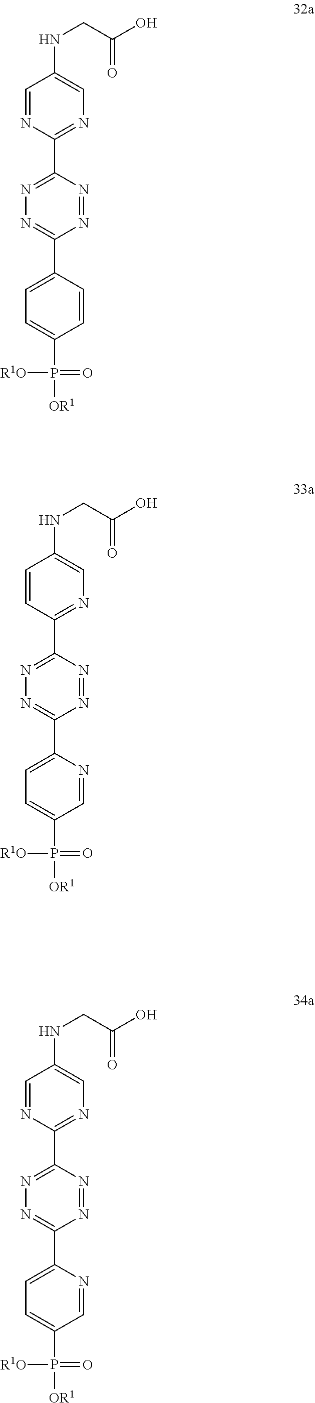

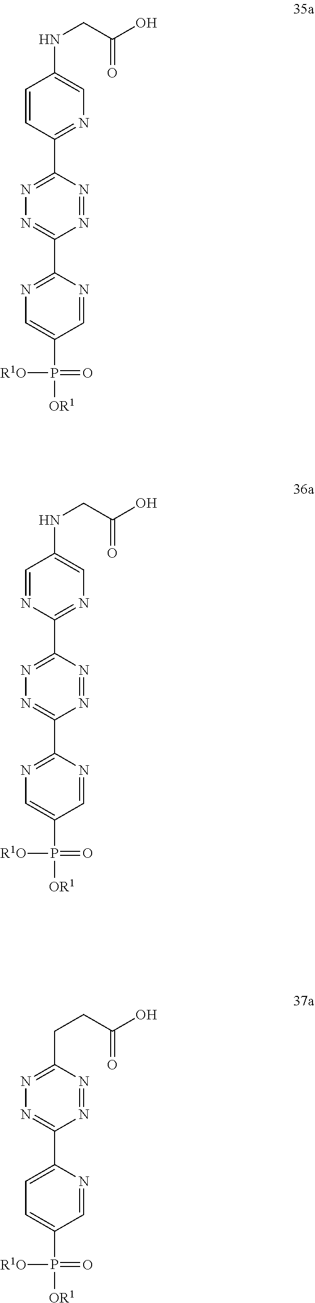

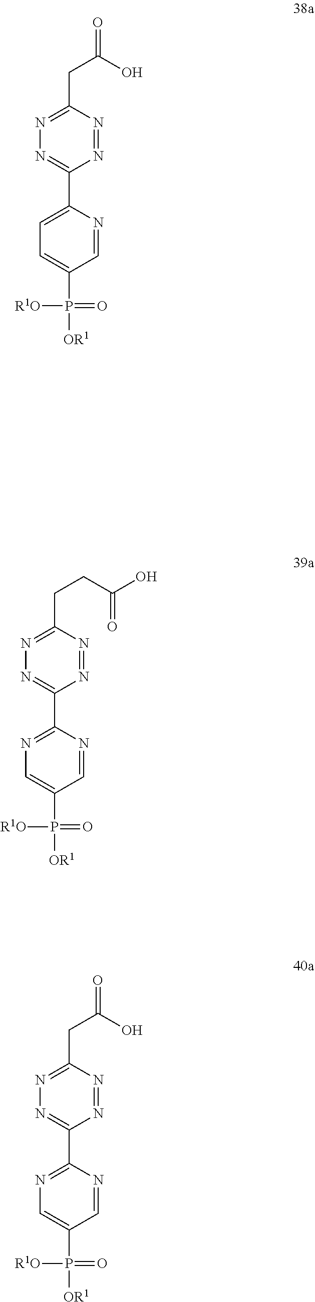

In the following table particular examples of synthesized intermediates of general formula II of the present invention are provided:

| Ex. | |
|---|---|
| No. | Formula |
| 1 | 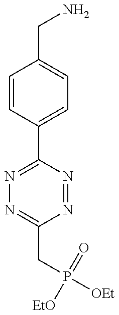 [SMILES: CCOP(=O)(Cc1nnc(-c2ccc(CN)cc2)nn1)OCC] |
| | 1 |
| 2 | 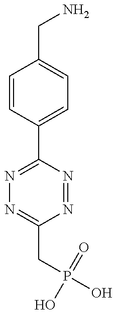 [SMILES: NCc1ccc(-c2nnc(CP(=O)(O)O)nn2)cc1] |
| | 2 |
| 3 | 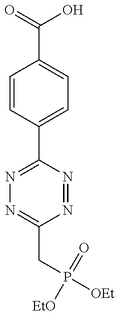 [SMILES: CCOP(=O)(Cc1nnc(-c2ccc(C(=O)O)cc2)nn1)OCC] |
| | 3 |
| 6 | 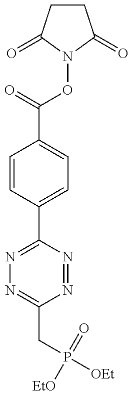 [SMILES: CCOP(=O)(Cc1nnc(-c2ccc(C(=O)ON3C(=O)CCC3=O)cc2)nn1)OCC] |
| | 6 |
| 12 | 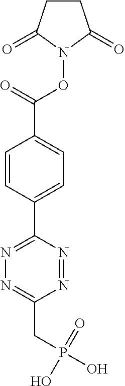 [SMILES: O=C(ON1C(=O)CCC1=O)c1ccc(-c2nnc(CP(=O)(O)O)nn2)cc1] |
| | 12 |
| 14 | 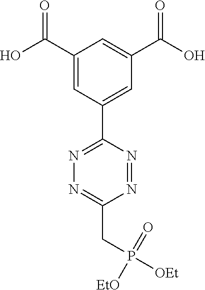 [SMILES: CCOP(=O)(Cc1nnc(-c2cc(C(=O)O)cc(C(=O)O)c2)nn1)OCC] |
| | 14 |
| 15 | 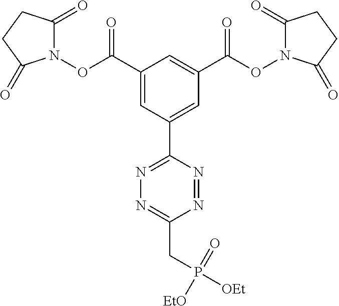 [SMILES: CCOP(=O)(Cc1nnc(-c2cc(C(=O)ON3C(=O)CCC3=O)cc(C(=O)ON3C(=O)CCC3=O)c2)nn1)OCC] |
| | 15 |
| 16 | 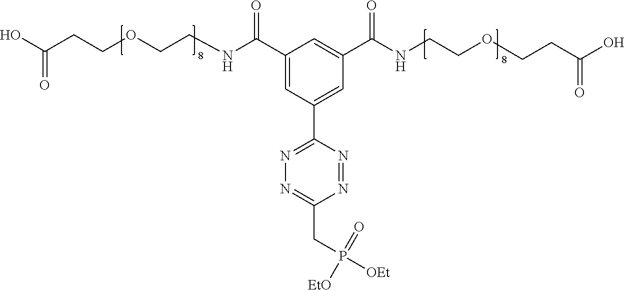 |
| | 16 |
| 17 | 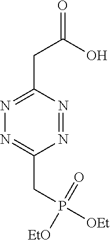 [SMILES: CCOP(=O)(Cc1nnc(CC(=O)O)nn1)OCC] |
| | 17 |
| 18 | 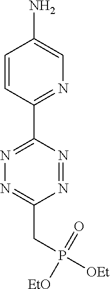 [SMILES: CCOP(=O)(Cc1nnc(-c2ccc(N)cn2)nn1)OCC] |
| | 18 |
| 19 | 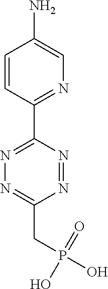 [SMILES: Nc1ccc(-c2nnc(CP(=O)(O)O)nn2)nc1] |
| | 19 |
| 20 | 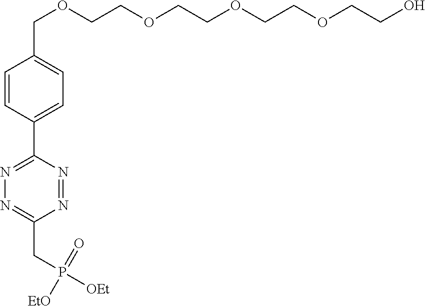 [SMILES: CCOP(=O)(Cc1nnc(-c2ccc(COCCOCCOCCOCCO)cc2)nn1)OCC] |
| | 20 |
| 21 | 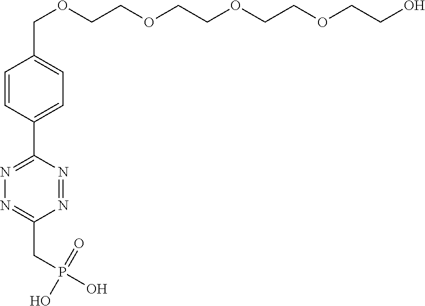 [SMILES: O=P(O)(O)Cc1nnc(-c2ccc(COCCOCCOCCOCCO)cc2)nn1] |
| | 21 |
| 22 | 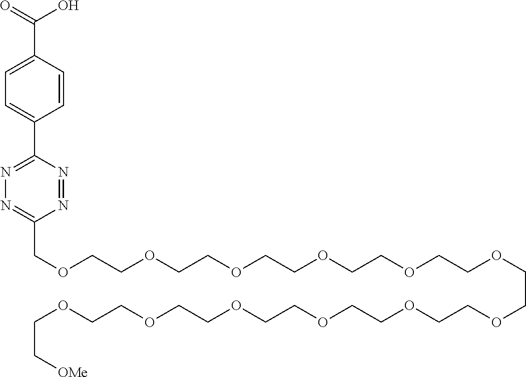 [SMILES: COCCOCCOCCOCCOCCOCCOCCOCCOCCOCCOCCOCCOCc1nnc(-c2ccc(C(=O)O)cc2)nn1] |
| | 22 |
| 24 | 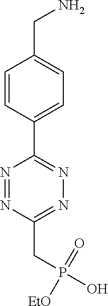 [SMILES: CCOP(=O)(O)Cc1nnc(-c2ccc(CN)cc2)nn1] |
| | 24 |
| 25 | 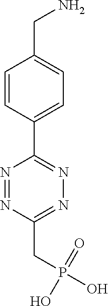 [SMILES: NCc1ccc(-c2nnc(CP(=O)(O)O)nn2)cc1] |
| | 25 |
| 26 | 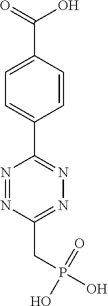 [SMILES: O=C(O)c1ccc(-c2nnc(CP(=O)(O)O)nn2)cc1] |
| | 26 |
| 30 | 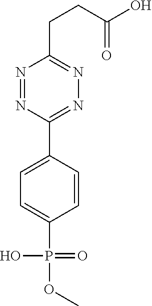 [SMILES: COP(=O)(O)c1ccc(-c2nnc(CCC(=O)O)nn2)cc1] |
| | 30 |
| 31 | 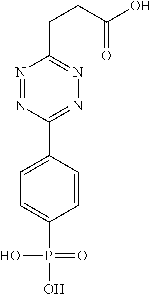 [SMILES: O=C(O)CCc1nnc(-c2ccc(P(=O)(O)O)cc2)nn1] |
| | 31 |
| 32 | 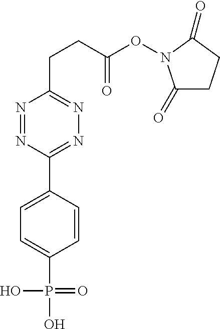 [SMILES: O=C(CCc1nnc(-c2ccc(P(=O)(O)O)cc2)nn1)ON1C(=O)CCC1=O] |
| | 32 |
| 33 | 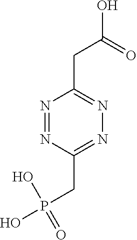 [SMILES: O=C(O)Cc1nnc(CP(=O)(O)O)nn1] |
| | 33 |
| 34 | 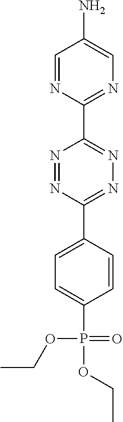 [SMILES: CCOP(=O)(OCC)c1ccc(-c2nnc(-c3ncc(N)cn3)nn2)cc1] |
| | 34 |
| 35 | 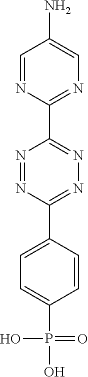 [SMILES: Nc1cnc(-c2nnc(-c3ccc(P(=O)(O)O)cc3)nn2)nc1] |
| | 35 |
| 36 | 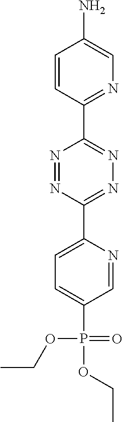 [SMILES: CCOP(=O)(OCC)c1ccc(-c2nnc(-c3ccc(N)cn3)nn2)nc1] |
| | 36 |
| 37 | 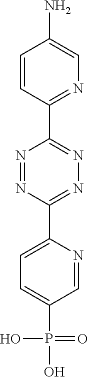 [SMILES: Nc1ccc(-c2nnc(-c3ccc(P(=O)(O)O)cn3)nn2)nc1] |
| | 37 |
| 38 | 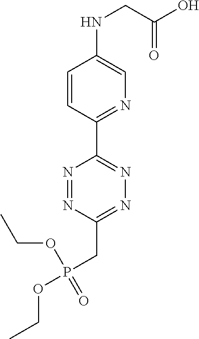 [SMILES: CCOP(=O)(Cc1nnc(-c2ccc(NCC(=O)O)cn2)nn1)OCC] |
| | 38 |
| 39 | 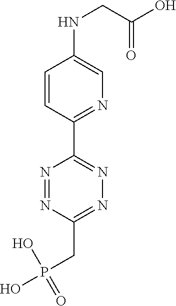 [SMILES: O=C(O)CNc1ccc(-c2nnc(CP(=O)(O)O)nn2)nc1] |
| | 39 |
| 48 | 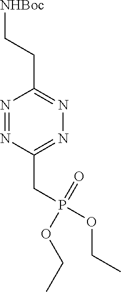 [SMILES: CCOP(=O)(Cc1nnc(CCNC(=O)OC(C)(C)C)nn1)OCC] |
| | 48 |
| 49 | 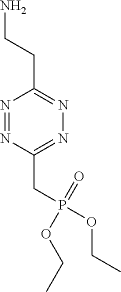 [SMILES: CCOP(=O)(Cc1nnc(CCN)nn1)OCC] |
| | 49 |
| 50 | 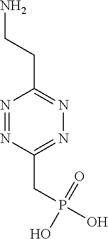 [SMILES: NCCc1nnc(CP(=O)(O)O)nn1] |
| | 50 |
| 51 | 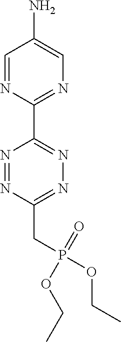 [SMILES: CCOP(=O)(Cc1nnc(-c2ncc(N)cn2)nn1)OCC] |
| | 51 |
| 52 | 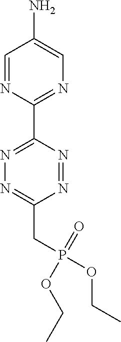 [SMILES: CCOP(=O)(Cc1nnc(-c2ncc(N)cn2)nn1)OCC] |
| | 52 |
| 53 | 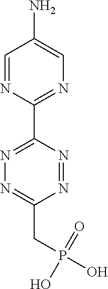 [SMILES: Nc1cnc(-c2nnc(CP(=O)(O)O)nn2)nc1] |
| | 53 |
| 55 | 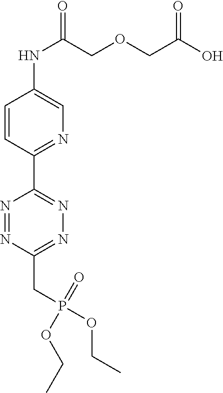 [SMILES: CCOP(=O)(Cc1nnc(-c2ccc(NC(=O)COCC(=O)O)cn2)nn1)OCC] |
| | 55 |
| 56 | 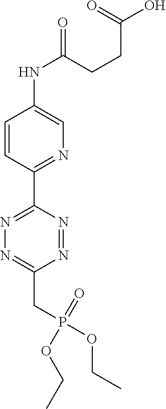 [SMILES: CCOP(=O)(Cc1nnc(-c2ccc(NC(=O)CCC(=O)O)cn2)nn1)OCC] |
| | 56 |
| 57 | 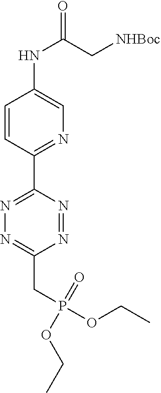 [SMILES: CCOP(=O)(Cc1nnc(-c2ccc(NC(=O)CNC(=O)OC(C)(C)C)cn2)nn1)OCC] |
| | 57 |
| 58 | 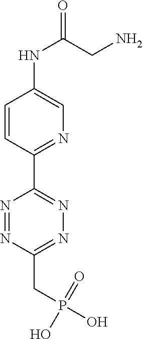 [SMILES: NCC(=O)Nc1ccc(-c2nnc(CP(=O)(O)O)nn2)nc1] |
| | 58 |
| 60 | 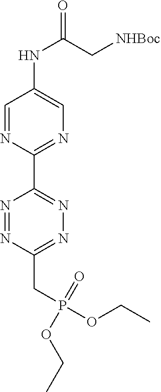 [SMILES: CCOP(=O)(Cc1nnc(-c2ncc(NC(=O)CNC(=O)OC(C)(C)C)cn2)nn1)OCC] |
| | 60 |
| 61 | 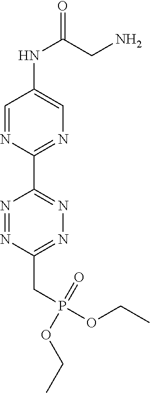 [SMILES: CCOP(=O)(Cc1nnc(-c2ncc(NC(=O)CN)cn2)nn1)OCC] |
| | 61 |
| 62 | 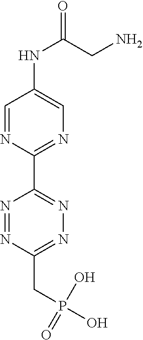 [SMILES: NCC(=O)Nc1cnc(-c2nnc(CP(=O)(O)O)nn2)nc1] |
| | 62 |
| 68 | 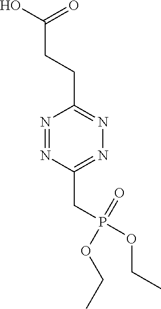 [SMILES: CCOP(=O)(Cc1nnc(CCC(=O)O)nn1)OCC] |
| | 68 |
| 69 | 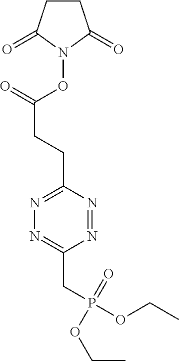 [SMILES: CCOP(=O)(Cc1nnc(CCC(=O)ON2C(=O)CCC2=O)nn1)OCC] |
| | 69 |
| 72 | 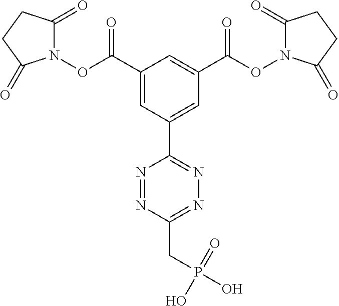 [SMILES: O=C(ON1C(=O)CCC1=O)c1cc(C(=O)ON2C(=O)CCC2=O)cc(-c2nnc(CP(=O)(O)O)nn2)c1] |
| | 72 |
| 75 | 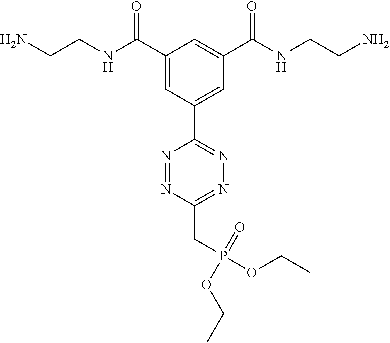 [SMILES: CCOP(=O)(Cc1nnc(-c2cc(C(=O)NCCN)cc(C(=O)NCCN)c2)nn1)OCC] |
| | 75 |
| 76 | 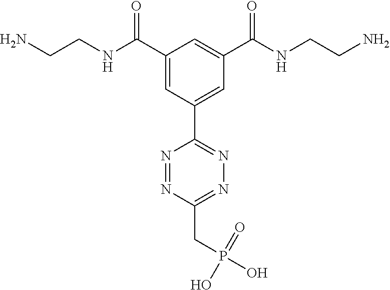 [SMILES: NCCNC(=O)c1cc(C(=O)NCCN)cc(-c2nnc(CP(=O)(O)O)nn2)c1] |
| | 76 |

### 5. The Fifth Aspect of the Present Invention

A fifth aspect of the present invention relates to methods of preparing certain tetrazine intermediates.

According to a twentieth embodiment of the present invention a method of preparing a tetrazine intermediate of general formula II, which method comprising the steps of:
i. reacting a first cyano compound of the general formula III

Z—Sp¹-CN (III)
wherein Z and Sp¹ are as defined above, wherein optionally any hydroxyl group of residue Z is provided in protected, i.p. alkoxy, form;
with a second cyano compound of the general formula IV

NC—Sp²-[R]ₙ₃ (IV)
wherein R and Sp² and n3 are as defined above
in the presence of a hydrazine hydrate;
ii. subsequent oxidation, in particular of an 1,4-dihydro-s-tetrazine compound formed in step 1, as for example with oxidants selected from NaNO₂, PhI(OAc)₂, DDQ, or air oxidation
iii. optionally isolating the obtained tetrazine compound;
and
iv. optionally deprotecting the hydroxyl groups of residue Z.

According to a particular embodiment thereof, step i) is performed in the absence of a catalyst.

According to another particular embodiment thereof, step i) is performed in the presence of a catalyst.

According to another particular embodiment thereof, the catalyst is a metal-containing catalyst, as for example Zn(OTf)₂.

According to another particular embodiment, the catalyst is a metal-free catalyst.

According to another particular embodiment, the metal-free catalyst is an organic catalyst.

According to another particular embodiment, the organic catalyst is a sulfur containing catalyst.

According to another particular embodiment the sulfur containing catalyst is selected from 3-mercaptopropionic acid, L-cysteine, glutathione, 2-aminoethanethiol, 1,3-propanedithiol, thioglycolic acid and N-acetyl-L-cysteine, and in particular 3-mercaptopropionic acid.

According to another particular embodiment the reaction step i) is carried out in a molar excess, as for example 1 to 20-fold molar excess, of the hydrazine compound over the compounds of formula (III) and (IV).

According to still another particular embodiment, the reaction step i) is carried out in an alcoholic solvent, in particular ethanol.

According to still another particular embodiment of the compounds of formula (III) and (IV) are employed in a molar ratio of 1:10 to 10:1.

According to another particular embodiment the reaction step ii) is carried out in a molar excess, as for example 1 to 20-fold molar excess, of the oxidant over the compounds of formula (III) and (IV).

According to still another particular embodiment the oxidant of step ii. is NaNO₂.

According to still another particular embodiment the oxidant of step ii. is PhI(OAc)₂

According to still another particular embodiment the oxidant of step ii. is air.

### 6. Further Aspects of the Invention

Further aspects of the present invention relate to the medical use of conjugates of the invention, pharmaceutical compositions and diagnostic or analytical kits containing the same.

According to a twenty-first embodiment of the present invention a conjugate as defined in anyone of the embodiments eleven to seventeen, for use in medicine, in particular for use in diagnosis and/or therapy is provided.

According to a twenty-second embodiment of the present invention a pharmaceutical composition is provided, comprising in a pharmaceutically acceptable carrier at least one conjugate as defined in anyone of the embodiments eleven to seventeen

According to a twenty-third embodiment of the present invention a diagnostic or analytical kit is provided, comprising at least one tetrazin compound as defined in anyone of the embodiments one to ten.

### D. Further Embodiments

### 1. Hydrophilic Tetrazine Intermediates and Preparation Thereof

The tetrazine intermediates of general formula II can be prepared in analogy to methods, which are well known in the art. Suitable methods are found in the various publications cited herein, all of which are incorporated herein by reference in their entireties. Some methods are outlined herein.

The metal-catalysed one-pot synthesis of tetrazine intermediates of formula II may be performed in line with the disclosure of Yang et al, Angew. Chem Int Ed (2012), 51, 5222.

Mao et al. describe in Angew. Chem Int Ed (2019), 58, 1106 the organocatalytic and scalable syntheses of unsymmetrical, 1,2,4,5-tetrazines by thiol-containing promoters.

According to a particular aspect of the present invention a method of preparing a tetrazine intermediate of general formula II

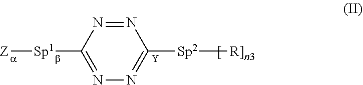

is provided.

In general, said which method comprising the steps of:
i) reacting a first cyano compound of the general formula III

Z—Sp¹-CN (III)
wherein Z and Sp¹ are as defined above, wherein optionally any hydroxyl group of residue Z is provided in protected, i.p. alkoxy, form.
with a second cyano compound of the general formula IV

NC—Sp²-[R]ₙ₃ (IV)
wherein R and Sp² and n3 are as defined above
in the presence of a hydrazine hydrate;
ii) subsequently oxidizing the product of step i)
iii) optionally isolating the obtained tetrazine compound;
   and
iv) optionally deprotecting any optionally protected hydroxyl group of residue Z.

More particularly, step i) is performed in a one-pot reaction. For this purpose, for example, a solution of the cyano educts of the general formulae (III) and (IV) is provided, which is supplemented with a hydrazine compound, in particular hydrazine hydrate, and optionally in the presence of a suitable catalyst.

Particular catalysts are acidic metal-free organo-catalysts mercapto compounds as for example 3-mercaptopropionic acid, L-cysteine, glutathione, 2-aminoethanethiol, 1,3-propanedithiol, thioglycolic acid and N-acetyl-L-cysteine, and in particular 3-mercaptopropionic acid. like 3-mercapto propionic acid. Alternative catalysts are metal-containing catalyst, as for example Zn(OTf)₂.

The reaction of step i) is performed under temperature control until completion. Subsequently any resulting dihydro tetrazine intermediate may be oxidized. For this purpose conventional oxidants, in particular sodium nitrite/HCl or nitric acid may be applied.

If required, the mixture of said cyano educts is provided in a suitable solvent. Typical solvents that may be used are selected from polar organic solvents, like THF and organic alcohols, in particular ethanol.

Typically, the compounds of formulae (III) and (IV) are provided in a molar ratio in the range of 1:10 to 10:1, as for example 1:5 to 5:1. More particularly the compound if formula III is provided in 1 to 10 or 1 to 5-fold molar excess

Typically, the hydrazine compound is added in molar excess over the cyano educts, as for example in a 1 to 20 or 4 to 15 fold excess over compound (III) or (IV)

Typically, the acid catalyst used in catalytic or, more particular, it we molar amounts relative to compound (III) or (IV).

Typically, the accident is applied in at least it we molar, more preferably in a molar excess over the hydrazine compound.

The reaction temperature is controlled in a range of −10 to +10° C.

The oxidation of step ii. converts the dihydrotetrazine intermediate as formed by step i) to the respective tetrazine. Usually the reaction is performed in the reaction mixture of step i) either in the presence of ambient air, or by the addition of a suitable oxidant, as for example NaNO₂, p-benzoquinone, DDQ, or PhI(OAc)₂. PhI(OAc)₂ as oxidant is describe for example by Selvaraj, R. et al in Tetrahedron Lett. 2014; 55(34): 4795-4797

As a non-limiting example the preparation of two different phosphate tetrazines is illustrated by the following scheme (the oxidation step is not explicitly shown):

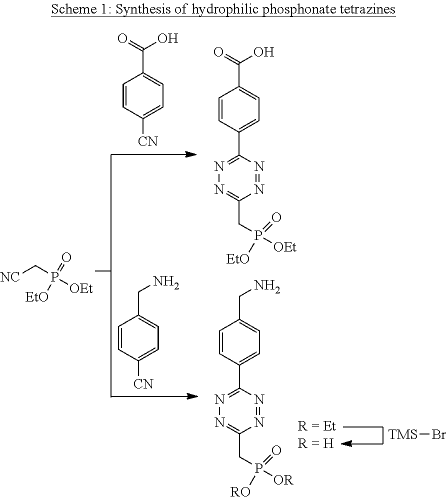

The optional step iii. for isolating the product of step ii. may be performed by means of conventional purification methods. Chromatographic methods, like flash chromatography or HPLC shall be particularly mentioned.

The process of the present invention may also comprise step iv., provided that an educt of formula III was applied, wherein residue Z comprises protected, for example esterified hydroxyl groups, and it is intended to make use of the produced tetrazine in deprotected form. Methods for the protecting esterified hydroxyl groups are well known. Deprotection by means of treatment with trimethylsilylbromide in organic solvent may be mentioned as non-limiting example.

By applying the above-mentioned general process particular derivatives of methylphosphonate tetrazines may be synthesized. These compounds contain functional groups allowing reactive with further chemical moieties, for example, through amide coupling or formation of carbamate or ester linkages. Such chemical moieties are selected from the above identified groups
Sp² spacer moiety (optionally in addition to a Sp² moiety already introduced as part of above reactant of formula IV)
A cleavable group
X self-immolative moiety
Y payload residue (cargo)
   or combinations thereof, as for example

### -A-X—Y, -A-Y, —X—Y, -A-X—X—Y, —X—X—Y

—Sp²-A-X—Y, —Sp²-A-Y, —Sp²-X—Y, —Sp²-A-X—X—Y

### 2. Payload Molecules Y

Payload molecules Y typically used as constituent of a tetrazine compound of the general formula I may be selected from Bioactive compounds, labeling agents, and chelators. Non-limiting examples thereof are given in the following sections.

### 2.1 Bioactive Compounds

Bioactive compounds include, but are not limited to, the following:

Bioactive compounds applicable according to the present invention include but are not limited to: small organic molecule drugs, steroids, lipids, proteins, aptamers, oligopeptides, oligonucleotides, oligosaccharides, as well as peptides, peptoids, amino acids, nucleotides, oligo- or polynucleotides, nucleosides, DNA, RNA, toxins, glycans and immunoglobulins.

Exemplary classes of bioactive compounds that can be used in the practice of the present invention include but are not limited to hormones, cytotoxins, antiproliferative/antitumor agents, antiviral agents, antibiotics, cytokines, anti-inflammatory agents, antihypertensive agents, chemosensitizing, photosensitizing and radiosensitizing agents, anti-AIDS substances, anti-viral agents, immunosuppressants, immunostimulants. enzyme inhibitors, anti-Parkinson agents, neurotoxins, channel blockers, modulators of cell-extracellular matrix interactions including cell growth inhibitors and anti-adhesion molecules, inhibitors of DNA, RNA or protein synthesis, steroidal and non-steroidal anti-inflammatory agents, anti-angiogenic factors, anti-Alzheimer agents.

In some embodiments, the bioactive compound is a low to medium molecular weight compound (e.g. about 200 to 5000 Da, about 200 to about 1500 Da, preferably about 300 to about 1000 Da).

Exemplary cytotoxic drugs are particularly those which are used for cancer therapy. Such drugs include, in general, DNA damaging agents, anti-metabolites, natural products and their analogs, enzyme inhibitors such as dihydro folate reductase inhibitors and thymidylate synthase inhibitors, DNA binders, DNA alkylators, radiation sensitizers, DNA intercalators, DNA cleavers, microtubule stabilizing and destabilizing agents, topoisomerases inhibitors. Examples include but are not limited to platinum-based drugs, the anthracycline family of drugs, the vinca drugs, the mitomycins, the bleomycins, the cytotoxic nucleosides, taxanes, lexitropsins, the pteridine family of drugs, diynenes, the podophyllotoxins, dolastatins, maytansinoids, differentiation inducers, and taxols. Particularly useful members of those classes include, for example, auristatins, maytansines, maytansinoids, calicheamicins, dactinomycines, duocarmycins, CC1065 and its analogs, camptothecin and its analogs, SN-38 and its analogs; DXd, tubulysin M, cryptophycins, pyrrolobenzodiazepines and pyrrolobenzodiazepine dimers (PBDs), pyridinobenzodiazepines (PDDs) and indolinobenzodiazepines (IBDs) (cf. US20210206763A1), methotrexate, methopterin, dichloromethotrexate, 5-fluorouracil, DNA minor groove binders, 6-mercaptopurine, cytosine arabinoside, melphalan, leurosine, leurosideine, actinomycin, anthracyclines (doxorubicin, epirubicin, idarubicin, daunorubicin, PNU-159682 (cf. U.S. Pat. No. 10,288,745 B2.) and its analogs, mitomycin C, mitomycin A, caminomycin, aminopterin, tallysomycin, podophyllotoxin and; podophyllotoxin derivatives such as etoposide or etoposide phosphate, vinblastine, vincristine, vindesine, taxol, taxotere retinoic acid, butyric acid, N8-acetyl spermidine, staurosporin, colchicine, camptothecin, esperamicin, ene-diynes, and their analogues, hemiasterlin and its analogues.

Other exemplary drug classes are angiogenesis inhibitors, cell cycle progression inhibitors, P13K/m-TOR/AKT pathway inhibitors, MAPK signaling pathway inhibitors, kinase inhibitors, protein chaperones inhibitors, HDAC inhibitors, PARP inhibitors, Wnt/Hedgehog signaling pathway inhibitors, RNA polymerase inhibitors, and protein degraders (cf. https://pubs.acs.org/doi/10.1021/acschembio.0c00285).

Examples of auristatins include dolastatin 10, monomethyl auristatin E (MMAE), auristatin F, monomethyl auristatin F (MMAF), auristatin F hydroxypropylamide (AF HPA), auristatin F phenylene diamine (AFP), monomethyl auristatin D (MMAD), auristatin PE, auristatin EB, auristatin EFP, auristatin TP and auristatin AQ. Suitable auristatins are also described in U.S. Publication Nos. 2003/0083263, 2011/0020343, and 2011/0070248; PCT Application; Publication Nos. WO09/117531, WO2005/081711, WO04/010957; WO02/088172 and WO01/24763, and U.S. Pat. Nos. 7,498,298; 6,884,869; 6,323,315; 6,239,104; 6,124,431; 6,034,065; 5,780,588; 5,767,237; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,879,278; 4,816,444; and 4,486,414, the disclosures of which are incorporated herein by reference in their entirety.

Exemplary drugs include the dolastatins and analogues thereof including: dolastatin A (U.S. Pat. No. 4,486,414), dolastatin B (U.S. Pat. No. 4,486,414), dolastatin 10 (U.S. Pat. Nos. 4,486,444, 5,410,024, 5,504,191, 5,521,284, 5,530,097, 5,599,902, 5,635,483, 5,663,149, 5,665,860, 5,780,588, 6,034,065, 6,323,315), dolastatin 13 (U.S. Pat. No. 4,986,988), dolastatin 14 (U.S. Pat. No. 5,138,036), dolastatin 15 (U.S. Pat. No. 4,879,278), dolastatin 16 (U.S. Pat. No. 6,239,104), dolastatin 17 (U.S. Pat. No. 6,239,104), and dolastatin 18 (U.S. Pat. No. 6,239,104), each patent incorporated herein by reference in their entirety.

Exemplary maytansines, maytansinoids, such as DM-1 and DM-4, or maytansinoid analogs, including maytansinol and maytansinol analogs, are described in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 6,441,163; 6,716,821 and 7,276,497.

Other examples include mertansine and ansamitocin. Pyrrolobenzodiazepines (PBDs), which expressly include dimers and analogs, include but are not limited to those described in [Denny, Exp. Opin. Ther. Patents, 10(4):459-474 (2000)], [Hartley et al., Expert Opin Investig Drugs. 2011, 20(6):733-44], Antonow et al., Chem Rev. 2011, 111(4), 2815-64].

Calicheamicins include, e.g. enediynes, esperamicin, and those described in U.S. Pat. Nos. 5,714,586 and 5,739,116.

Examples of duocarmycins and analogs include CC1065, duocarmycin SA, duocarmycin A, duocarmycin B I, duocarmycin B2, duocarmycin CI, duocarmycin C2, duocarmycin D, DU-86, KW-2189, adozelesin, bizelesin, carzelesin, seco-adozelesin. Other examples include those described in, for example, U.S. Pat. Nos. 5,070,092; 5,101,092; 5,187,186; 5,475,092; 5,595,499; 5,846,545; 6,534,660; 6,548,530; 6,586,618; 6,660,742; 6,756,397; 7,049,316; 7,553,816; 8,815,226; US20150104407; 61/988,011 filed May 2, 2014 and 62/010,972 filed Jun. 11, 2014; the disclosure of each of which is incorporated herein in its entirety.

Exemplary vinca alkaloids include vincristine, vinblastine, vindesine, and navelbine, and those disclosed in U.S. Publication Nos. 2002/0103136 and 2010/0305149, and in U.S. Pat. No. 7,303,749, the disclosures of which are incorporated herein by reference in their entirety.

Exemplary epothilone compounds include epothilone A, B, C, D, E, and F, and derivatives thereof. Suitable epothilone compounds and derivatives thereof are described, for example, in U.S. Pat. Nos. 6,956,036; 6,989,450; 6,121,029; 6,117,659; 6,096,757; 6,043,372; 5,969,145; and 5,886,026; and WO97/19086; WO98/08849; WO98/22461; WO98/25929; WO98/38192; WO99/01124; WO99/02514; WO99/03848; WO99/07692; WO99/27890; and WO99/28324; the disclosures of which are incorporated herein by reference in their entirety.

Exemplary cryptophycin compounds are described in U.S. Pat. Nos. 6,680,311 and; 6,747,021; the disclosures of which are incorporated herein by reference in their entirety.

Exemplary platinum compounds include cisplatin, carboplatin, oxaliplatin, iproplatin, ormaplatin, tetraplatin.

Exemplary DNA binding or alkylating drugs include CC-1065 and its analogs, anthracyclines, calicheamicins, dactinomycines, mitromycines, pyrrolobenzodiazepines, and the like.

Exemplary microtubule stabilizing and destabilizing agents include taxane compounds, such as paclitaxel, docetaxel, tesetaxel, and carbazitaxel; maytansinoids, auristatins and analogs thereof, vinca alkaloid derivatives, epothilones and cryptophycins.

Exemplary topoisomerase inhibitors include camptothecin and camptothecin derivatives, camptothecin analogs and non-natural camptothecins, such as, for example, CPT-11, SN-38, topotecan, 9-aminocamptothecin, rubitecan, gimatecan, karenitecin, silatecan, lurtotecan, exatecan, DXd, diflomotecan, belotecan, lurtotecan and S39625. Other camptothecin compounds that can be used in the present invention include those described in, for example, J. Med. Chem., 29:2358-2363 (1986); J. Med. Chem., 23:554 (1980); J. Med Chem., 30:1774 (1987).

Angiogenesis inhibitors include, but are not limited to, MetAP2 inhibitors, VEGF inhibitors, PIGF inhibitors, VGFR inhibitors, PDGFR inhibitors, MetAP2 inhibitors. Exemplary VGFR and PDGFR inhibitors include sorafenib, sunitinib and vatalanib. Exemplary MetAP2 inhibitors include fumagillol analogs, meaning compounds that include the fumagillin core structure.

Exemplary cell cycle progression inhibitors include CDK inhibitors such as, for example, BMS-387032 and PD0332991; Rho-kinase inhibitors such as, for example, AZD7762; aurora kinase inhibitors such as, for example, AZD1152, MLN8054 and MLN8237; PLK inhibitors such as, for example, BI 2536, B16727, GSK461364, ON-01910; and KSP inhibitors such as, for example, SB 743921, SB 715992, MK-0731, AZD8477, AZ3146 and ARRY-520.

Exemplary P13K/m-TOR/AKT signalling pathway inhibitors include phosphoinositide 3-kinase (P13K) inhibitors, GSK-3 inhibitors, ATM inhibitors, DNA-PK inhibitors and PDK-1 inhibitors.

Exemplary P13 kinases are disclosed in U.S. Pat. No. 6,608,053, and include BEZ235, BGT226, BKM120, CAL263, demethoxyviridin, GDC-0941, GSK615, IC87114, LY294002, Palomid 529, perifosine, PF-04691502, PX-866, SAR245408, SAR245409, SF1126, Wortmannin, XL147 and XL765.

Exemplary AKT inhibitors include, but are not limited to AT7867.

Exemplary MAPK signaling pathway inhibitors include MEK, Ras, JNK, B-Raf and p38 MAPK inhibitors.

Exemplary MEK inhibitors are disclosed in U.S. Pat. No. 7,517,944 and include GDC-0973, GSKI 120212, MSC1936369B, AS703026, R05126766 and R04987655, PD0325901, AZD6244, AZD8330 and GDC-0973.

Exemplary B-raf inhibitors include CDC-0879, PLX-4032, and SB590885.

Exemplary B p38 MAPK inhibitors include BIRB 796, LY2228820 and SB 202190. Exemplary receptor tyrosine kinases inhibitors include but are not limited to AEE788 (NVP-AEE 788), BIBW2992 (Afatinib), Lapatinib, Erlotinib (Tarceva), Gefitinib (Iressa), AP24534 (Ponatinib), ABT-869 (linifanib), AZD2171, CHR-258 (Dovitinib), Sunitinib (Sutent), Sorafenib (Nexavar), and Vatalinib.

Exemplary protein chaperon inhibitors include HSP90 inhibitors. Exemplary inhibitors include 17AAG derivatives, B11B021, B11B028, SNX-5422, NVP-AUY-922 and KW-2478.

Exemplary HDAC inhibitors include Belinostat (PXD101), CUDC-101, Droxinostat, ITF2357 (Givinostat, Gavinostat), JNJ-26481585, LAQ824 (NVP-LAQ824, Dacinostat), LBH-589 (Panobinostat), MC1568, MGCD0103 (Mocetinostat), MS-275 (Entinostat), PCI-24781, Pyroxamide (NSC 696085), SB939, Trichostatin A and Vorinostat (SAHA). Exemplary PARP inhibitors include iniparib (BSI 201), olaparib (AZD-2281), ABT-888 (Veliparib), AG014699, CEP9722, MK 4827, KU-0059436 (AZD2281), LT-673, 3-aminobenzamide, A-966492, and AZD2461.

Exemplary Wnt/Hedgehog signalling pathway inhibitors include vismodegib, cyclopamine and XAV-939.

Exemplary RNA polymerase inhibitors include amatoxins. Exemplary amatoxins include alpha-amanitins, beta amanitins, gamma amanitins, eta amanitins, amanullin, amanullic acid, amanisamide, amanon, and proamanullin.

Exemplary cytokines include IL-2, IL-7, IL-10, IL-12, IL-15, IL-21, TNF.

As non-limiting examples of particular drugs there may be mentioned Auristatins, Maytansinoids, PBDs, topoisomerase inhibitors, anthracyclines

In another embodiment, a combination of two or more different drugs as described above are used.

According to another embodiment, the bioactive compound may be selected from any synthetic or naturally occurring compounds comprising one or more natural and/or non-natural, proteinogenic and/or non-proteinogenic amino acid residues, such as in particular oligo- or polypeptides or proteins.

A particular group of such compounds comprises immunoglobulin molecules as for example antibodies, antibody derivatives, antibody fragments, antibody (fragment) fusions (e.g. bi-specific and tri-specific mAb fragments or derivatives), polyclonal or monoclonal antibodies, such as human, humanized, mouse or chimeric antibodies.

Typical non-limiting examples of antibodies for use in the present invention are selected form biologically, in particular pharmacologically active antibody molecules. Non-limiting examples are selected form the following group: trastuzumab, bevacizumab, cetuximab, panitumumab, ipilimumab, rituximab, alemtuzumab, ofatumumab, gemtuzumab, brentuximab, ibritumomab, tositumomab, pertuzumab, adecatumumab, IGN101, INA01 labetuzumab, hua33, pemtumomab, oregovomab, minretumomab (CC49), cG250, J591, MOv-18, farletuzumab (MORAb-003), 3F8, ch14,18, KW-2871, hu3S193, IgN31 1, IM-2C6, CDP-791, etaracizumab, volociximab, nimotuzumab, MM-121, AMG 102, METMAB, SCH 900105, AVE1642, IMC-A12, MK-0646, R1507, CP 751871, KB004, III A4, mapatumumab, HGS-ETR2, CS-1008, denosumab, sibrotuzumab, F19, 81 C6, pinatuzumab, lifastuzumab, glembatumumab, coltuximab, lorvotuzumab, indatuximab, anti-PSMA, MLN-0264, ABT-414, milatuzumab, ramucirumab, abagovomab, abituzumab, adecatumumab, afutuzumab, altumomab pentetate, amatuximab, anatumomab, anetumab, apolizumab, arcitumomab, ascrinvacumab, atezolizumab, bavituximab, bectumomab, belimumab, bivatuzumab, brontictuzumab, cantuzumab, capromab, catumaxomab, citatuzumab, cixutumumab, clivatuzumab, codrituzumab, conatumumab, dacetuzumab, dallotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elgemtumab, emactuzumab, enavatuzumab emibetuzumab, enfortumab, enoblituzumab, ensituximab, epratuzumab, ertumaxomab, etaracizumab, farletuzumab, ficlatuzumab, figitumumab, flanvotumab, futuximab, galiximab, ganitumab, icrucumab, igovomab, imalumab, imgatuzumab, indusatumab, inebilizumab, intetumumab, iratumumab, isatuximab, lexatuzumab, lilotomab, lintuzumab, lirilumab, lucatumumab, lumretuzumab, margetuximab, matuzumab, mirvetuximab, mitumomab, mogamulizumab, moxetumomab, nacolomab, naptumomab, namatumab, necitumumab, nesvacumab, nimotuzumab, nivolumab, nofetumomab, obinutuzumab, ocaratuzumab, ofatumumab, olaratumab, onartuzumab, ontuxizumab, oportuzumab, oregovomab, otiertuzumab, pankomab, parsatuzumab, pasotuxizumab, patritumab, pembrolizumab, pemtumomab, pidilizumab, pintumomab, polatuzumab, pritumumab, quilizumab, racotumomab, ramucirumab, rilotumumab, robatumumab, sacituzumab, samalizumab, satumomab, seribantumab, siltuximab, sofituzumab, tacatuzumab, taplitumomab, tarextumab, tenatumomab, teprotumumab, tetulomab, ticilimumab, tigatuzumab, tositumomab, tovetumab, tremelimumab, tucotuzumab, ublituximab, ulocuplumab, urelumab, utomilumab, vadastuximab, vandortuzumab, vantictumab, vanucizumab, varlilumab, veltuzumab, vesencumab, volociximab, vorsetuzumab votumumab, zalutumumab, zatuxima, combination and derivatives thereof, as well as other monoclonal antibodies targeting CAI 25, CAI 5-3, CAI 9-9, L6, Lewis Y, Lewis X, alpha fetoprotein, CA 242, placental alkaline phosphatase, prostate specific antigen, prostate specific membrane antigen, prostatic acid phosphatase, epidermal growth factor, MAGE-1, MAGE-2, MAGE-3, MAGE-4, transferrin receptor, p97, MUCI, CEA, gpIOO, MARTI, IL-2 receptor, CD20, CD52, CD33, CD22, human chorionic gonadotropin, CD38, CD40, mucin, P21, MPG, and Neu oncogene product.

### 2.2 Labelling Agents

Labeling agents which may be used according to the invention can comprise any type of label known in the art which does not inhibitor negatively affect reactivity of the tetrazine moiety.

Labels of the invention include, but are not limited to, dyes (e.g. fluorescent, luminescent, or phosphorescent dyes, such as dansyl, coumarin, fluorescein, acridine, rhodamine, silicon-rhodamine, BODIPY, or cyanine dyes), chromophores (e.g., phytochrome, phycobilin, bilirubin, etc.), radiolabels (e.g. radioactive forms of hydrogen, fluorine, carbon, phosphorous, sulphur, or iodine, such as tritium, fluorine-18, carbon-11, carbon-14, phosphorous-32, phosphorous-33, sulphur-33, sulphur-35, iodine-123, or iodine-125), MRI-sensitive spin labels, affinity tags (e.g. biotin, His-tag, Flag-tag, strep-tag, sugars, lipids, sterols, PEG-linkers, benzylguanines, benzylcytosines, or co-factors), polyethylene glycol groups (e.g., a branched PEG, a linear PEG, PEGs of different molecular weights, etc.), photocrosslinkers (such as p-azidoiodoacetanilide), NMR probes, X-ray probes, pH probes, IR probes, resins, solid supports.

In some embodiments, exemplary dyes can include an NIR contrast agent that fluoresces in the near infrared region of the spectrum. Exemplary near-infrared fluorophores can include dyes and other fluorophores with emission wavelengths (e.g., peak emission wavelengths) between about 630 and 1000 nm, e.g., between about 630 and 800 nm, between about 800 and 900 nm, between about 900 and 1000 nm, between about 680 and 750 nm, between about 750 and 800 nm, between about 800 and 850 nm, between about 850 and 900 nm, between about 900 and 950 nm, or between about 950 and 1000 nm. Fluorophores with emission wavelengths (e.g., peak emission wavelengths) greater than 1000 nm can also be used in the methods described herein.

In some embodiments, exemplary fluorophores include 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-diethylaminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.) and ATTO dyes.

Other suitable fluorophores are for example described in EP3572468A1. Further labelling agents are 177-Lutetium, 89-Zirkonium, 131-Iod, 68-Gallium, 99m-Technetium, 225-Actinium, 213-Bismuth, 90-Yttrium, 212-Plumbum, 111-Indium, 64-Copper, 67-Copper, 124-Iodine, 227-Thorium and 188-Rhenium.

### 2.3 Chelators

Lists of typically applicable chelators and their short names are given below; Corresponding salts thereof are also applicable.

Acetyl acetone (ACAC), ethylene diamine (EN), 2-(2-aminoethylamino)ethanol (AEEA), diethylene triamine (DIEN), iminodiacetate (IDA), triethylene tetramine (TRIEN), triaminotriethylamine, nitrilotriacetate (NTA) and its salts like Na₃NTA or FeNTA, ethylenediaminotriacetate (TED), ethylenediamine tetraacetate (EDTA) and its salts like Na₂EDTA and CaNa₂EDTA, diethylene triaminpentaacetate (DTPA), 1,4,7,10-ztetraazacyclododecane-1,4,7,10-tetraacetate (DOTA), 1,4,7-triazacyclononane-1,4,7-triacetic acid (NOTA), Oxalate (OX), tartrate (TART), citrate (CIT), dimethylglyoxime (DMG), 8-hydroxyquinoline, 2,2-bipyridine (BPY), 1,10-phenanthroline (PHEN), dimercapto succinic acid (DMSA), 1,2-bis(diphenylphosphino)ethane (DPPE), sodium salicylate, methoxy salicylates, British anti-Lewisite or 2,3-dimercaprol (BAL), meso-2,3-dimercaptosuccinic acid (DMSA); Siderophores secreted by microorganisms, as for example desferrioxamine or deferoxamine B, also known as Deferral (Novartis), produced by Streptomyces spp.; deferoxamine (DFO), a trihydroxamic acid secreted by Streptomyces pilosus; phytochemicals like curcuminoids and derivatives of mugineic acid, like 3-hydroxy-mugineic acid and 2′-deoxy-mugineic acid; synthetically produced chelators, like Ibuprofen; derivatives of catechol, hydroxamate and hydroxypyridinone, like hydroxamate desferal and hydroxypyridinone deferiprone; deferiprone (L1 or 1,2-dimethyl-3-hydroxypyrid-4-one); D-penicillamine (DPA or D-PEN) whoich is A-s-dimethylcysteine or 3-mercapto-D-valine; tetraethylenetetraamine (TETA) or trientine and its two major metabolites N₁-acetyltriethylenetetramine (MAT) and N₁,N₁₀-diacetyltriethylenetetramine (DAT); hydroxyquinolines; clioquinol, which is a halogenated derivative of 8-hydroxyquinoline; and 5,7-dichloro-2-[(dimethylamino)methyl]quinolin-8-ol (PBT2)

### 2.4 Photosensitizer/Protein Degraders

As non-limiting examples there may be mentioned PROTACs in general, but there is a plethora of different E3 ligase binding molecules in combination with specific targeted proteins to degrade (vgl WO2017201449A1). (Maneiro, M. et al ACS Chem. Biol. 2020, 15, 6, 1306-1312

### 3. Attachment of Payload Molecules Y to Hydrophilic Tetrazines

### 3.1 Cleavable Moieties A

Suitable cleavable moieties A are well known from the prior art.

Reference can be made to:
Bargh et al., Chem Soc Rev 2019, 48(16), 4361-4374; Poreba, FEBS J 2020, 287(10), 1936-1969 and Salomon et al., Mol Pharmaceutics 2019 16, (12), 4817-4825;

As further examples there may be mentioned β-glucuronide linkers, carrying a beta-glucuronic acid derived trigger residue.

Non-limiting examples thereof are:

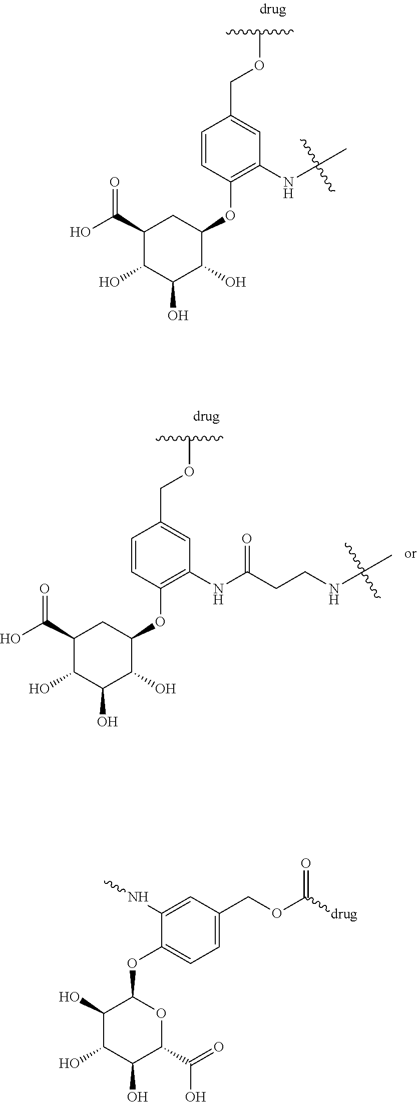

### 3.2 Self-Immolative Groups X

Suitable self-immolative moieties X are well known from the prior art.

Reference can be made to: Santi et al., J Med Chem 2014, 57(6), 2303-2314; Alouane et al., Angew Chem Int Ed 2015, 54(26), 7492-7509; and Kolakowski et al., Angew Chem 2016, 128(28), 8080-8083

### 4. (Bio-) Conjugate Formation

A bio-conjugate of the present invention is provided by bioconjugation, wherein a suitably functionalized biomolecule is reacted with a tetrazine compound of formula I in order to form a covalent linkage between said functionalized biomolecule and said tetrazine. Said biomolecule acts as “targeting agent” which targets the payload moiety Y, which is part of the tetrazine compound of formula I, to a particular place of interest (“target”). Depending to the type of such “target” a respective suitable “targeting agent” can be selected. Once targeted, the bio-conjugate comprising the payload moiety may then further act on the target or the biological structure comprising said target.

4.1 Targeting Agents and their Targets

The primary object of such targeting agent is the formation of a covalent or noncovalent linkage with a particular “target”. A secondary object of the targeting agent is the targeted transport of a “payload molecule” to said target In order to achieve said second object the POI has to be combined (reversibly or irreversibly) with at least one payload molecule. For this purpose said POI has to be functionalised by introducing said at least one ncAA. The functionalized POI carrying said at least one ncAA may then be linked to said at least one payload molecule through bioconjugation via said ncAA residue. Said ncAA is reactive with a payload molecule which in turn carries a corresponding moiety reactive with said at least one ncAA residue of the POI. The thus obtained bioconjugate, i.e. the targeting agent, allows the transfer of the payload molecule to the intended target.

For example, a “target” can be any molecule, which is present in and/or on an organism, tissue or cell. Such targets may be nonspecific or specific for a particular organism, tissue or cell. Targets include cell surface targets, e.g. receptors, glycoproteins, glycans, carbohydrates; structural proteins, e.g. amyloid plaques; abundant extracellular targets such as in stroma, extracellular matrix targets such as growth factors, and proteases; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof.

Examples of targets include compounds such as proteins of which the presence or expression level is correlated with a certain tissue or cell type or of which the expression level is up-regulated or down-regulated in a certain disorder.

In particular, such target is a protein such as a (internalizing or non-internalizing) receptor.

Targets can be selected from any suitable targets within the human or animal body or on a pathogen or parasite.

Non-limiting examples of suitable targets include but are not limited to a group comprising cellular components such as cell membranes and cell walls, receptors such as cell membrane receptors, intracellular structures such as Golgi bodies or mitochondria, enzymes, receptors, DNA, RNA, viruses or viral particles, macrophages, tumor-associated macrophages, antibodies, proteins, carbohydrates, monosaccharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-) angiogenesis marker, gastrin receptor, dopaminergic system, serotonergic system, GABAergic system, adrenergic system, cholinergic system, opioid receptors, GPIIb/IIIa receptor and other thrombus related receptors, fibrin, calcitonin receptor, tuftsin receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase, integrin receptor, fibronectin targets, AOC3, ALK, AXL, C242, CA-125, CCL11, CCR5, CD2, CD3, CD4, CD5, CD15, CA15-3, CD18, CD19, CA19-9, CD20, CD21, CD22, CD23, CD25, CD28, CD30, CD31, CD33, CD37, CD38, CD40, CD41, CD44v6, CD45, CD51, CD52, CD54, CD56, CD62E, CD62P, CD62L, CD70, CD72, CD74, CD79-B, CD80, CD105, CD125, CD138, CD141, CD147, CD152, CD154, CD174, CD227, CD326, CD340, VEGF/EGF and VEGF/EGF receptors, VEGF-A, VEGFR2, VEGFR1, TAG72, CEA, MUC1, MUC16, GPNMB, PSMA, Cripto, Tenascin C, Melanocortin-1 receptor, G250, HLA DR, ED-B, TMEFF2, EphB2, EphB4, EphA2, FAP, Mesothelin, GD2, GD3, CAIX, 5T4, clumping factor, CTLA-4, CXCR2, FGFRI, FGFR2, FGFR3, FGFR4, NaPi2b, NOTCHI, NOTCH2, NOTCH3, NOTCH4, ErbB2, ErbB3, EpCAM, FLT3, HGF, HER2, HER3, HM124, ICAM, ICOS-L, IGF-1 receptor, TRPV1, CFTR, gdNMB, CA9, c-KIT, c-MET, ACE, APP, adrenergic receptor beta2, Claudine 3, RON, RORI, PD-LI, PD-L2, B7-H3, B7-H4, IL-2 receptor, IL-4 receptor, IL-13 receptor, integrins, IFN-alpha, IFN-gamma, IgE, IGF-1 receptor, IL-1, IL-4, IL-5, IL-6, IL-12, IL-13, IL-22, IL-23, interferon receptor, ITGB2 (CD18), LFA-1 (CDI Ia), L-selectin, P-selectin, E-selectin, mucin, myostatin, NCA-90, NGF, PDGFR alpha, prostatic carcinoma cells, Pseudomonas aeruginosa, rabies, RANKL, respiratory syncytial virus, Rhesus factor, SLAMF7, sphingosine-1-phosphate, TGF-1, TGFbeta2, TGFbeta, TNFalpha, TRAIL-R1, TRAIL-R2, CTAA 16.88, vimentin, matrix metalloproteinases (MMP) such as MMP2, MMP9, MMP14, LDL receptor, endoglins, polysialic acids and their corresponding lectins. An example of fibronectin targets are the alternatively spliced extra-domain-A (ED-A) and extra-domain-B (ED-B) of fibronectin. Non-limiting examples of targets in stroma can be found in V. Hofmeister, D. Schrama, J. C. Becker, Cancer Immun. Immunother. 2008, 57, 1, the contents of which are hereby incorporated by reference.

More particularly, in order to allow a (specific) targeting of the above-listed targets, the targeting agent can comprise compounds comprising an ncAA-functionalized peptide sequence. Such compounds include but are not limited to antibodies, antibody derivatives, antibody fragments, antibody (fragment) fusions (e.g. bi-specific and tri-specific mAb fragments or derivatives), proteins, peptides, e.g. octreotide and derivatives, VIP, MSH, LHRH, chemotactic peptides, bombesin, elastin, peptide mimetics, receptor agonists and antagonists, cytokines, hormones, steroids, toxins.

According to a particular aspect, the target is a receptor and a targeting agent is employed, which is capable of specific binding to the target. Suitable targeting agents include but are not limited to, the ligand of such a receptor or a part thereof, which still binds to the receptor, e.g. a receptor binding peptide in the case of receptor binding protein ligands.

Other examples of targeting agents of protein nature include insulin, transferrin, fibrinogen-gamma fragment, thrombospondin, claudin, apolipoprotein E, Affibody molecules such as for example ABY-025, Ankyrin repeat proteins, ankyrin-like repeat proteins, interferons, e.g. alpha, beta, and gamma interferon, interleukins, lymphokines, colony stimulating factors and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin.

Examples of peptides molecules, like antibody, as used in targeting agents include LHRH receptor targeting peptides, EC-1 peptide, RGD peptides, HER2-targeting peptides, PSMA targeting peptides, somatostatin-targeting peptides, bombesin. Other examples of targeting agents include lipocalins, such as anticalins.

One particular embodiment uses Affibodies™ and multimers and derivatives.

In one particular embodiment, antibodies are used to form a targeting agent. While antibodies or immunoglobulins derived from IgG antibodies are particularly well-suited for use in this invention, immunoglobulins from any of the classes or subclasses may be selected, e.g. IgG, IgA, IgM, IgD and IgE. Suitably, the immunoglobulin is of the class IgG including but not limited to IgG subclasses (IgG1, 2, 3 and 4) or the class IgM which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, camelized single domain antibodies, recombinant antibodies, anti-idiotype antibodies, multispecific antibodies, antibody fragments, such as, Fv, VHH, Fab, F(ab)2, Fab′, Fab′-SH, F(ab′)2, single chain variable fragment antibodies (scFv), tandem/bis-scFv, Fc, pFc′, scFv-Fc, disulfide Fv (dsFv), bispecific antibodies (bc-scFv) such as BiTE antibodies, trispecific antibody derivatives such as tribodies, camelid antibodies, minibodies, nanobodies, resurfaced antibodies, humanized antibodies, fully human antibodies, single domain antibodies (sdAb, also known as Nanobody™), chimeric antibodies, chimeric antibodies comprising at least one human constant region, dual-affinity antibodies such as dual-affinity retargeting proteins (DART™), and multimers and derivatives thereof, such as divalent or multivalent single-chain variable fragments (e.g. di-scFvs, tri-scFvs) including but not limited to minibodies, diabodies, triabodies, tribodies, tetrabodies, and the like, and multivalent antibodies. Reference is made to [Trends in Biotechnology 2015, 33, 2, 65], [Trends Biotechnol. 2012, 30, 575-582], and [Cane. Gen. Prot. 2013 10, 1-18], and [BioDrugs 2014, 28, 331-343], the contents of which are hereby incorporated by reference.

“Antibody fragment” refers to at least a portion of the variable region of the immunoglobulin that binds to its target, i.e. the antigen-binding region.

Other embodiments use antibody mimetics as targeting agents, such as but not limited to Affimers, Anticalins, Avimers, Alphabodies, Affibodies, DARPins, and multimers and derivatives thereof; reference is made to [Trends in Biotechnology 2015, 33, 2, 65], the contents of which is hereby incorporated by reference.

For the avoidance of doubt, in the context of this invention the term “antibody” is meant to encompass all of the antibody variations, fragments, derivatives, fusions, analogs and mimetics outlined in this paragraph, unless specified otherwise.

In a preferred embodiment the targeting agent is selected from agents derived from antibodies and antibody derivatives such as antibody fragments, fragment fusions, proteins, peptides, peptide mimetics.

In another preferred embodiment the targeting agent is selected from agents derived from antibody fragments, fragment fusions, and other antibody derivatives that do not contain a Fc domain.

Typical non-limiting examples of antibody molecules to be further modified to form ncAA modified targeting agents of the present invention are selected form biologically, in particular pharmacologically active antibody molecules. Non-limiting examples are selected form the following group: trastuzumab, bevacizumab, cetuximab, panitumumab, ipilimumab, rituximab, alemtuzumab, ofatumumab, gemtuzumab, brentuximab, ibritumomab, tositumomab, pertuzumab, adecatumumab, IGN101, INA01 labetuzumab, hua33, pemtumomab, oregovomab, minretumomab (CC49), cG250, J591, MOv-18, farletuzumab (MORAb-003), 3F8, ch14,18, KW-2871, hu3S193, IgN31 1, IM-2C6, CDP-791, etaracizumab, volociximab, nimotuzumab, MM-121, AMG 102, METMAB, SCH 900105, AVE1642, IMC-A12, MK-0646, R1507, CP 751871, KB004, III A4, mapatumumab, HGS-ETR2, CS-1008, denosumab, sibrotuzumab, F19, 81 C6, pinatuzumab, lifastuzumab, glembatumumab, coltuximab, lorvotuzumab, indatuximab, anti-PSMA, MLN-0264, ABT-414, milatuzumab, ramucirumab, abagovomab, abituzumab, adecatumumab, afutuzumab, altumomab pentetate, amatuximab, anatumomab, anetumab, apolizumab, arcitumomab, ascrinvacumab, atezolizumab, bavituximab, bectumomab, belimumab, bivatuzumab, brontictuzumab, cantuzumab, capromab, catumaxomab, citatuzumab, cixutumumab, clivatuzumab, codrituzumab, conatumumab, dacetuzumab, dallotuzumab, daratumumab, demcizumab, denintuzumab, depatuxizumab, derlotuximab, detumomab, dinutuximab, drozitumab, duligotumab, durvalumab, dusigitumab, ecromeximab, edrecolomab, elgemtumab, emactuzumab, enavatuzumab emibetuzumab, enfortumab, enoblituzumab, ensituximab, epratuzumab, ertumaxomab, etaracizumab, farletuzumab, ficlatuzumab, figitumumab, flanvotumab, futuximab, galiximab, ganitumab, icrucumab, igovomab, imalumab, imgatuzumab, indusatumab, inebilizumab, intetumumab, iratumumab, isatuximab, lexatuzumab, lilotomab, lintuzumab, lirilumab, lucatumumab, lumretuzumab, margetuximab, matuzumab, mirvetuximab, mitumomab, mogamulizumab, moxetumomab, nacolomab, naptumomab, namatumab, necitumumab, nesvacumab, nimotuzumab, nivolumab, nofetumomab, obinutuzumab, ocaratuzumab, ofatumumab, olaratumab, onartuzumab, ontuxizumab, oportuzumab, oregovomab, otiertuzumab, pankomab, parsatuzumab, pasotuxizumab, patritumab, pembrolizumab, pemtumomab, pidilizumab, pintumomab, polatuzumab, pritumumab, quilizumab, racotumomab, ramucirumab, rilotumumab, robatumumab, sacituzumab, samalizumab, satumomab, seribantumab, siltuximab, sofituzumab, tacatuzumab, taplitumomab, tarextumab, tenatumomab, teprotumumab, tetulomab, ticilimumab, tigatuzumab, tositumomab, tovetumab, tremelimumab, tucotuzumab, ublituximab, ulocuplumab, urelumab, utomilumab, vadastuximab, vandortuzumab, vantictumab, vanucizumab, varlilumab, veltuzumab, vesencumab, volociximab, vorsetuzumab votumumab, zalutumumab, zatuxima, combination and derivatives thereof, as well as other monoclonal antibodies targeting CAI 25, CAI 5-3, CAI 9-9, L6, Lewis Y, Lewis X, alpha fetoprotein, CA 242, placental alkaline phosphatase, prostate specific antigen, prostate specific membrane antigen, prostatic acid phosphatase, epidermal growth factor, MAGE-1, MAGE-2, MAGE-3, MAGE-4, transferrin receptor, p97, MUCI, CEA, gpIOO, MARTI, IL-2 receptor, CD20, CD52, CD33, CD22, human chorionic gonadotropin, CD38, CD40, mucin, P21, MPG, and Neu oncogene product.

According to a further particular embodiment of the invention, the target and targeting agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting targets with tissue-, cell- or disease-specific expression.

By way of example, the targeting agent specifically binds or complexes with a cell surface molecule, such as a cell surface receptor or antigen, for a given cell population. Following specific binding or complexing of the targeting agent with the receptor, the drug will enter the cell.

As used herein, a targeting agent that “specifically binds or complexes with” or “targets” a cell surface molecule, an extracellular matrix target, or another target, preferentially associates with the target via intermolecular forces. For example, the ligand can preferentially associate with the target with a dissociation constant (Kd or KD) of less than about 50 nM, less than about 5 nM, or less than about 500 pM.

### 4.3 Dienophiles

The targeting agents, in particular biomolecules, normally have to be functionalised in order to enable the covalent binding of a tetrazine compound of the general formula I.

Methods of functionalisation are well known in the art.

Different classes of dienophiles suitable for reacting with a tetrazine moiety are well known in the art. In general, more- or poly-cyclic, in particular mono- and bi-cyclic unsaturated dienophile are applicable.

Such dienophiles are capable of reaction via a Diels-Alder-type cycloaddition reaction, as for example cyclooctynyl-dienophiles, trans-cyclooctenyl-dienophiles, norbornenyl dienophiles, cyclopropenyl dienophiles, cyclobutenyl dienophiles, spirohexenyl dienophiles, BCN dienophiles, or azetine dienophiles.

They are described in the prior art, as for example (the disclosure of which is herewith incorporated by reference):
Oliveira, B. L. et al, Chem Soc Rev 2017, 46, 4895;
Kozma, E., ChemBioChem 2017,18, 486;
Siegl, S. J. et al, Chem Eur J 2018, 24, 2426;
Ramil, C. P. et al, J Am Chem Soc 2017, 139, 13376
Liu, K, et al Chem Comm, 2017, 53, 10604
WO 2015/107064 A1 in the name of European Molecular Biology Laboratory.
WO2012104422 A1
US2013137763A1

### 4.4 Bioorthogonal Bioconjugation

A targeting agent, in particular a targeting agent comprising a polypeptide portion, comprising one or more than one UNAA residue can be prepared according to the present invention using a suitable translation system, in particular in vivo translation system. An in vivo translation system can be a cell, e.g. a prokaryotic or eukaryotic cell. The cell can be a bacterial cell, e.g. E. coli; a fungal cell such as a yeast cell, e.g. S. cerevisiae or a methylotrophic yeast; a plant cell, or an animal cell such as an insect cell or a mammalian cell, e.g. a HEK cell or a HeLa cell. Eukaryotic cells used for polypeptide expression may be single cells or parts of a multicellular organism.

The applied cellular system comprises (e.g., is fed with) at least one unnatural amino acid or a salt thereof corresponding to the UNAA residue(s) of the targeting agent to be prepared. The cellular system further comprises:
(i) a PyIRS of the invention and a tRNA^{PyI}, wherein the PyIRS is capable of (preferably selectively) acylating the tRNA^{PyI} with the UNAA or salt thereof; and
(ii) a polynucleotide encoding the targeting agent, wherein any position of the targeting agent, occupied by an UNAA residue is encoded by a codon (e.g. selector codon) that is the reverse complement of the anticodon of the tRNA^{PyI}.

The cellular system is cultured so as to allow translation of the targeting agent, encoding polynucleotide (ii), thereby producing the targeting agent.

For producing a targeting agent, according to a method of the present invention, the translation in step (b) can be achieved by culturing the cellular system under suitable conditions, preferably in the presence of (e.g., in a culture medium containing) the UNAA or salt thereof, for a time suitable to allow translation at a ribosome of the cell. Depending on the polynucleotide(s) encoding the targeting agent, (and optionally the PyIRS, tRNA^{PyI}), it may be required to induce expression by adding a compound inducing transcription, such as, e.g., arabinose, isopropyl β-D-thiogalactoside (IPTG) or tetracycline. mRNA that encodes the targeting agent, (and comprises one or more than codon that is the reverse complement of the anticodon comprised by the tRNA^{PyI}) is bound by the ribosome. Then, the polypeptide is formed by stepwise attachment of amino acids and UNAAs at positions encoded by codons which are recognized (bound) by respective aminoacyl tRNAs. Thus, the UNAA(s) is/are incorporated in the targeting agent, at the position(s) encoded by the codon(s) that is/are the reverse complement of the anticodon comprised by the tRNA^{PyI}.

The cellular system may comprise a polynucleotide sequence encoding the PyIRS of the invention which allows for expression of the PyIRS by the cell. Likewise, the tRNA^{PyI} may be produced by the cellular system based on a tRNA^{PyI}-encoding polynucleotide sequence comprised by the cell. The PyIRS-encoding polynucleotide sequence and the tRNA^{PyI}-encoding polynucleotide sequence can be located either on the same polynucleotide or on separate polynucleotides.

Thus, in one embodiment, the present invention provides a method for producing a targeting agent, comprising one or more than one UNAA residue, wherein the method comprises the steps of:
(a) providing a cellular system comprising polynucleotide sequences encoding:
   at least one PyIRS of the invention,
   at least one tRNA (tRNA^{PyI}) that can be acylated by the PyIRS, and
   at least one targeting agent, wherein any position of the targeting agent, occupied by an UNAA residue is encoded by a codon that is the reverse complement of the anticodon of the tRNA^{PyI}; and
(b) allowing for translation of the polynucleotide sequences by the cellular system in the presence of an UNAA or a salt thereof, thereby producing the PyIRS, tRNA^{PyI} and the POI.

The cellular system used for preparing a targeting agent, comprising one or more than one unnatural amino acid residue as described herein can be prepared by introducing polynucleotide sequences encoding the PyIRS, the tRNA^{PyI} and the targeting agent, into a (host) cell. Said polynucleotide sequences can be located on the same polynucleotide or on separate polynucleotides, and can be introduced into the cell by methods known in the art (such as, e.g., using virus-mediated gene delivery, electroporation, microinjection, lipofection, or others).

After translation, the targeting agent, prepared according to the present invention may optionally be recovered and purified, either partially or substantially to homogeneity, according to procedures generally known in the art. Unless the targeting agent, is secreted into the culture medium, recovery usually requires cell disruption. Methods of cell disruption are well known in the art and include physical disruption, e.g., by (ultrasound) sonication, liquid-sheer disruption (e.g., via French press), mechanical methods (such as those utilizing blenders or grinders) or freeze-thaw cycling, as well as chemical lysis using agents which disrupt lipid-lipid, protein-protein and/or protein-lipid interactions (such as detergents), and combinations of physical disruption techniques and chemical lysis. Standard procedures for purifying polypeptides from cell lysates or culture media are also well known in the art and include, e.g., ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in making correctly folded mature proteins. High performance liquid chromatography (HPLC), affinity chromatography or other suitable methods can be employed in final purification steps where high purity is desired. Antibodies made against the polypeptides of the invention can be used as purification reagents, i.e. for affinity-based purification of the polypeptides. A variety of purification/protein folding methods are well known in the art, including, e.g., those set forth in Scopes, Protein Purification, Springer, Berlin (1993); and Deutscher, Methods in Enzymology Vol. 182: Guide to Protein Purification, Academic Press (1990); and the references cited therein.

As noted, those of skill in the art will recognize that, after synthesis, expression and/or purification, polypeptides can possess a conformation different from the desired conformations of the relevant polypeptides. For example, polypeptides produced by prokaryotic systems often are optimized by exposure to chaotropic agents to achieve proper folding. During purification from, e.g., lysates derived from E. coli, the expressed polypeptide is optionally denatured and then renatured. This is accomplished, e.g., by solubilizing the proteins in a chaotropic agent such as guanidine HCl. In general, it is occasionally desirable to denature and reduce expressed polypeptides and then to cause the polypeptides to re-fold into the preferred conformation. For example, guanidine, urea, DTT, DTE, and/or a chaperonin can be added to a translation product of interest. Methods of reducing, denaturing and renaturing proteins are well known to those of skill in the art. Polypeptides can be refolded in a redox buffer containing, e.g., oxidized glutathione and L-arginine.

The targeting agent thus prepared may then be converted to a respective bioconjugate by reaction with a tetrazine compound of the above general formula I

### 5. Pharmaceutical Compositions

The conjugates or bioconjugates of the present invention, as for example APCs, in particular ADCs (i.e. the active agents or ingredients) of this invention are generally given as “pharmaceutical compositions” comprised of a therapeutically and/or prophylactically effective amount or a diagnostically effective amount of at least one such active ingredient or its pharmaceutically acceptable salt and optionally at least one pharmaceutically acceptable excipient.

Said pharmaceutical compositions may be delivered via suitable routes of administration such as via oral, rectal, transmucosal, topical, ophthalmic, otologic, or intestinal administration; parenteral delivery, including intramuscular, subcutaneous, intramedullary injections, as well as intrathecal, direct intraventricular, intravenous, intraperitoneal, intranasal, or intraocular injections, as the case may be.

Depending on the nature or the mode of administration and dosage form said composition said at least one additional pharmaceutical excipient may be different.

An “excipient” is a substance formulated alongside the active ingredient and is included for different purpose, as for example for long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as for example facilitating drug absorption, reducing viscosity, or enhancing solubility. Excipients can also be useful in the manufacturing process of the pharmaceutical composition, to aid in the handling of the active substance concerns such as by facilitating powder flowability or non-stick properties, in addition to aiding in vitro stability such as prevention of denaturation or aggregation over the expected shelf life. The selection of appropriate excipients not only depends upon the route of administration and the dosage form, but also on the particular active ingredient and other factors.

Excipients may be selected from the following classes: immunological adjuvants, antiadherents, binders, coatings, colours, disintegrant, flavours, glidants, lubricants, preservatives, sorbents, sweeteners, and vehicles.

Non limiting examples of excipients comprise diluents, preserving agents, stabilizers, emulsifying agents, like emulsifying polymers, such as polysorbates or poloxamers, antioxidants; anti-irritants, chelating agents and stabilizing salts, such as chlorides, sulfates, phosphates, diphosphates, hydrobromides and nitrates, suspending agents, antibacterial agents or antifungal agents. Further, buffering agents such as buffering systems of low molecular weight organic acids together with the respective salts, or inorganic buffering substances, such as phosphate buffers, can be used. Further suitable ingredients are also known from relevant pharmacological standard literature. Also the proportion of the various components will vary depending on the nature of the specific component used and is generally known to the person skilled in the art (Remington's Pharmaceutical science (“Handbook of Pharmaceutical Excipients”, 2nd Edition, (1994), Edited by A Wade and P J Weller or in Remington's Pharmaceutical Sciences, Mack Publishing Co. (A. R Gennaro edit. 1985).

A pharmaceutical composition as used herein may be presented in the form of a “dosage form” or “unit dose” and may comprise one or more APC, in particular ADCs as described herein. Thus, a pharmaceutical composition as used herein could, for example, provide two active agents admixed together in a unit dose or provide two active agents combined in a dosage form wherein the active agents are physically separated.

Furthermore, one may administer the pharmaceutical composition in a targeted drug delivery system, for example, in a liposome coated with endothelial cell-specific antibody.

The pharmaceutical compositions of the present invention may be manufactured in a manner that is itself known, e.g., by means of conventional mixing, dissolving, emulsifying, encapsulating, entrapping or combinations thereof. Proper formulation is dependent upon the route of administration chosen.

The phrase “pharmaceutically acceptable” is employed herein to refer to those compounds, materials, compositions, and/or dosage forms which are, within the scope of sound medical judgment, suitable for use in contact with the tissues of patients without excessive toxicity, irritation, allergic response, or other problem or complication commensurate with a reasonable risk/benefit ratio.

The invention includes all “pharmaceutically acceptable salt forms” of the active ingredient. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

A “therapeutically effective amount” and/or “prophylactically effective amount” means an amount effective, when administered to a human or non-human patient, to provide any therapeutic and/or prophylactic benefit. More particularly, a “therapeutically effective amount” is an amount of an active ingredient disclosed herein or a combination of two or more such active ingredients, which inhibits, totally or partially, the progression of the condition or alleviates, at least partially, one or more symptoms of the condition.

A “diagnostically effective amount” means an amount effective to allow obtaining from the patient a diagnostically valuable information on status or progression of a disease state.

A therapeutic benefit may be an amelioration of symptoms of a diseased patient, e.g., an amount effective to decrease the symptoms of a diseased patient. In certain circumstances a patient may not present symptoms of a condition for which the patient is being treated. Thus, a prophylactically effective amount of a compound is also an amount sufficient to provide a significant positive effect on any indicia of a disease, disorder or condition e.g. an amount sufficient to significantly reduce the frequency and severity of disease symptoms to occur.

A therapeutically effective amount can also be an amount, which is prophylactically effective.

A “patient” as used herein means human or non-human, in particular human, animals.

A “dosage form” is any unit of administration (“unit dose”) of one or more active agents as described herein.

The term “treating” or “treatment” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition. In particular it encompasses a prophylactic or therapeutic treatment or combinations thereof.

“Frequency” of dosage may vary depending on the compound used and the particular type of infection treated. A dosage regimen of once per day is possible. Dosage regimens in which the active agent is administered for several times daily, as for example 2 to 10 times, like 2, 3, 4, 5, 6, 7, 8, 9 or 10 times may occasionally be more helpful.

It will be understood, however, that the specific dose level and frequency for any particular patient will depend upon a variety of factors including the activity of the specific compound employed, the age, body weight, general health, sex, diet, time of administration, route of administration, rate of excretion, drug combination and the severity of the particular disease in the patient undergoing therapy. Patients may generally be monitored for therapeutic or prophylactic effectiveness using assays suitable for the condition being treated or prevented, which will be familiar to those of ordinary skill in the art.

Particular examples of pharmaceutical compositions according to the present invention are liquid form preparations such as solutions, suspensions, and emulsions and comprise, a therapeutically effective amount of at least one APC, in particular ADC component as defined above, optionally together with at least one further pharmaceutically acceptable excipient as defined above and may be administered through any suitable route.

Further examples of pharmaceutical compositions according to the present invention are solid form preparations such as powders, tablets, pills, capsules, cachets, suppositories, and dispersible granules.

The numerous possible variations that will become immediately evident to a person skilled in the art after heaving considered the disclosure provided herein also fall within the scope of the invention.

The following examples are illustrative only and are not intended to limit the scope of the embodiments described herein.

### Examples

### A) Materials and Methods

Reagents were purchased from commercial suppliers and used without further purification. All solvents, including anhydrous solvents, were used as obtained from the commercial sources. Air and water-sensitive reagents and reactions were generally handled under argon atmosphere.

The reaction progress was monitored by TLC on Merck silica gel plates 60 F254 or via UHPLC-MS. TLC-detection was executed either via UV-light at 254 nm or with potassium permanganate staining.

Flash chromatographic purification was performed on a Biotage Isolera One purification system using silica gel (0.060-0.200 mm), KP-Sil cartridges.

Preparative HPLC purification was performed on Agilent Infinity 1260 series equipment consisting of Agilent 1260 preparative pumps, a 1260 preparative autosampler, a 1260 fraction collector and a 1260 multiple wavelength detector VL. The preparative column used was a Waters X-Bridge Prep C18 column: 5 μm; 19×150 mm operated with a linear gradient of H₂O and acetonitrile, both containing 0.1% TFA as solvents.

Nuclear magnetic resonance spectra were recorded on a Bruker Avance (400 MHz) NMR System at room temperature. Chemical shifts (6) are given in parts per million (ppm), coupling constants (J) given in Hertz (Hz) and multiplicity is reported using standard abbreviations.

UHPLC-MS analyses were performed on Agilent Infinity 1290 series equipment consisting of an Agilent 1290 quaternary pump, a 1290 sampler, a 1290 thermostated column compartment and a 1290 Diode array detector VL+ equipped with a quadrupole LC/MS 6120 and an Infinity 1260 ELSD. The analytical column used was an Acquity UPLC BEH C18 column: 1.7 μm; 2.1×50 mm operated with a linear gradient of H₂O and acetonitrile, both containing 0.1% TFA as solvents.

### B) Synthesis of Compounds

### Example 1—Synthesis of diethyl ((6-(4-(aminomethyl)phenyl)-1,2,4,5-tetrazin-3-yl)methyl)phosphonate (1)

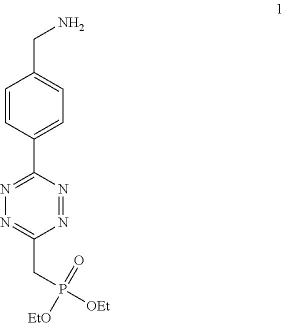 [SMILES: CCOP(=O)(Cc1nnc(-c2ccc(CN)cc2)nn1)OCC]

To a solution of 4-aminomethyl-benzonitrile (169 mg, 1.00 mmol, 1.00 eq) and diethyl cyanomethyl-phosphonate (651 μL, 709 mg, 4.00 mmol, 4.00 eq) in EtOH (0.5 mL) was added 3-mercaptopropionic acid (87.0 μL, 106 mg, 1.00 mmol, 1.00 eq) followed by hydrazine monohydrate (776 μL, 801 mg, 16.0 mmol, 16.0 eq) at 0° C. The mixture was stirred at room temperature over night. Afterwards NaNO₂ (1.38 g, 20.0 mmol, 20.0 eq) in H₂O was added and it was acidified to pH˜3 via dropwise addition of 1 M HCl_{aq}.

Purification via HPLC yielded 1 as pink oil (300 mg) which was directly employed for the next step.

### Example 2—Synthesis of ((6-(4-(aminomethyl)phenyl)-1,2,4,5-tetrazin-3-yl)methyl)phosphonic Acid (2)

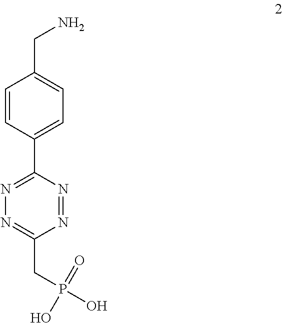 [SMILES: NCc1ccc(-c2nnc(CP(=O)(O)O)nn2)cc1]

To a solution of 1 (300 mg) in DMF was added trimethylsilyl bromide (530 μL, 614 mg, 4.01 mmol, 5.00 eq) at 0° C. The mixture was stirred at room temperature over night. Afterwards it was diluted with MeOH and H₂O and the solvents evaporated.

Purification via HPLC yielded 2 as a pink powder (30.0 mg, 8% over two steps).

### Example 3—Synthesis of 4-(6-((diethoxyphosphoryl)methyl)-1,2,4,5-tetrazin-3-yl)benzoic Acid (3)

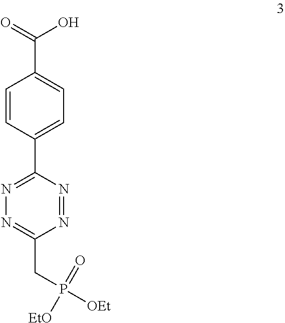 [SMILES: CCOP(=O)(Cc1nnc(-c2ccc(C(=O)O)cc2)nn1)OCC]

To a solution of 4-cyanobenzoic acid (588 mg, 4.00 mmol, 1.00 eq) and diethyl cyanomethyl-phosphonate (2.60 mL, 2.84 g, 16.0 mmol, 4.00 eq) in EtOH (2 mL) was added 3-mercaptopropionic acid (348 μL, 424 mg, 4.00 mmol, 1.00 eq) followed by hydrazine monohydrate (3.10 mL, 3.20 g, 64.0 mmol, 16.0 eq) at 0° C. The mixture was stirred at room temperature over night. Afterwards NaNO₂ (5.52 g, 80.0 mmol, 20.0 eq) in H₂O was added and it was acidified to pH˜3 via dropwise addition of 1 M HCl_{aq}. The pink precipitate was filtered and washed with 0.1 M HCl_{aq}.

Purification via flash chromatography (dichloromethane/MeOH 20:1) yielded 3 as pink powder (304 mg, 22%).

### Example 4—Synthesis of 2(6-(dimethylamino)-3-(dimethyliminio)-3H-xanthen-9-yl)-5-((4-(6-(phosphonomethyl)-1,2,4,5-tetrazin-3-yl)benzyl)carbamoyl)benzoate (4)

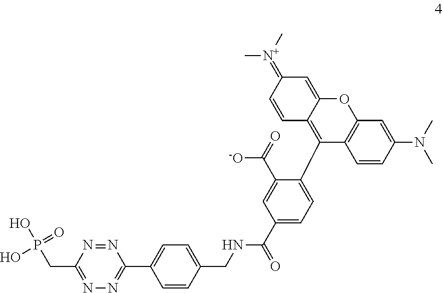

To a solution of 2 (5.00 mg, 17.8 μmol, 1.00 eq) in DMF (0.2 mL) was added DIPEA (12.4 μL, 9.20 mg, 71.2 μmol, 4.00 eq) and 5-TAMRA-OSu (10.3 mg, 19.6 μmol, 1.10 eq) and the mixture was stirred at room temperature over night. The crude reaction mixture was directly subjected to purification via HPLC.

Purification via HPLC yielded 4 as red-brown powder (4.0 mg, 32%).

### Example 5—Synthesis of ((6-(4-(((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamoyl)phenyl)-1,2,4,5-tetrazin-3-yl)methyl)phosphonic Acid (5)

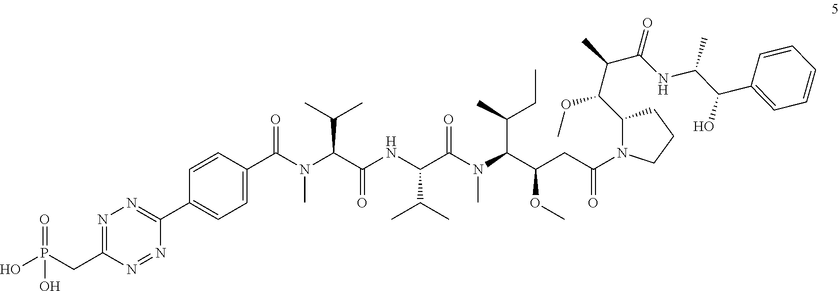 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)c1ccc(-c2nnc(CP(=O)(O)O)nn2)cc1)C(C)C]

To a solution of 3 (5.57 mg, 15.8 μmol, 1.00 eq) was added DIPEA (6.88 μL, 5.11 mg, 39.5 μmol, 2.50 eq) and HATU (9.01 mg, 23.7 μmol, 1.50 eq) and the mixture stirred at room temperature for 30 min. Afterwards MMAE (12.5 mg, 17.4 μmol, 1.10 eq) was added and the mixture stirred at room temperature over night. It was diluted with dichloromethane, washed with H₂O and the organic phase dried and the solvent evaporated under reduced pressure. The crude product was dissolved in DMF (0.5 mL) and it was added trimethylsilyl bromide (10.4 μL, 12.1 mg, 79.0 μmol, 5.00 eq) at 0° C. The mixture was stirred at room temperature over night and directly subjected to purification via HPLC.

Purification via HPLC yielded 5 as pink powder (5.0 mg, 32% over 2 steps).

### Example 6—Synthesis of 2,5-dioxopyrrolidin-1-yl 4-(6-((diethoxyphosphoryl)methyl)-1,2,4,5-tetrazin-3-yl)benzoate (6)

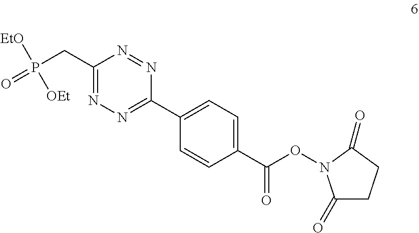 [SMILES: CCOP(=O)(Cc1nnc(-c2ccc(C(=O)ON3C(=O)CCC3=O)cc2)nn1)OCC]

To a solution of 3 (100 mg, 284 μmol, 1.00 eq) in dichloromethane (2.8 mL) was added N-hydroxysuccinimide (49.0 mg, 426 μmol, 1.50 eq) and EDC hydrochloride (81.6 mg, 426 μmol, 1.50 eq) and the mixture was stirred at room temperature for 1 h.

Purification via flash chromatography (dichloromethane/MeOH 20:1) yielded 6 as pink solid (118 mg, 93%).

### Example 7—Synthesis of (4-(6-((diethoxyphosphoryl)methyl)-1,2,4,5-tetrazin-3-yl)benzoyl)-L-valyl-L-alanine (7)

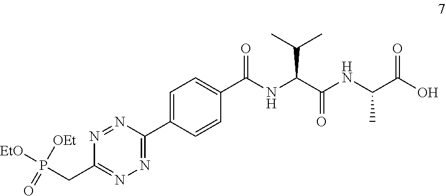 [SMILES: CCOP(=O)(Cc1nnc(-c2ccc(C(=O)N[C@H](C(=O)N[C@@H](C)C(=O)O)C(C)C)cc2)nn1)OCC]

To a solution of 6 (118 mg, 263 μmol, 1.00 eq) in DMF (2.6 mL) was added H-Val-Ala-OH (74.1 mg, 394 μmol, 1.50 eq) and NEt₃ (72.8 μL, 525 μmol, 2.00 eq) and the mixture was stirred at room temperature It was diluted with dichloromethane and washed with H₂O and 1 M HCl_{aq}. The organic phase was dried and the solvent evaporated under reduced pressure. Purification via flash chromatography (dichloromethane/MeOH 20:1→10:1) yielded 7 as pink solid (135 mg, 98%).

### Example 8—Synthesis of diethyl ((6-(4-(((S)-1-(((S)-1-(4-(hydroxymethyl)phenyl)amino)-1-oxopropan-2-yl)amino)-3-methyl-1-oxobutan-2-yl)carbamoyl)phenyl)-1,2,4,5-tetrazin-3-yl)methyl)phosphonate (8)

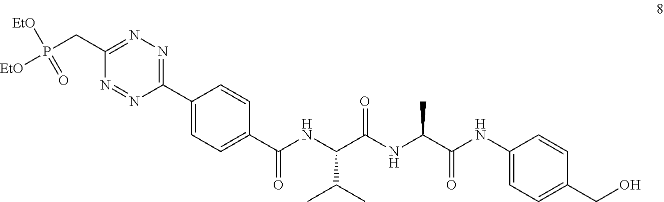 [SMILES: CCOP(=O)(Cc1nnc(-c2ccc(C(=O)N[C@H](C(=O)N[C@@H](C)C(=O)Nc3ccc(CO)cc3)C(C)C)cc2)nn1)OCC]

To a solution of 7 (116 mg, 222 μmol, 1.00 eq) in DMF (2.2 mL) was added p-aminobenzyl alcohol (41.0 mg, 333 μmol, 1.50 eq), HATU (127 mg, 333 μmol, 1.50 eq) and DIPEA (96.7 μL, 555 μmol, 2.50 eq) and the mixture was stirred at room temperature It was diluted with dichloromethane and washed with H₂O and 1 M HCl_{aq}. The organic phase was dried and the solvent evaporated under reduced pressure.

Purification via flash chromatography (dichloromethane/MeOH 20:1→10:1) yielded 8 as pink solid (131 mg, 94%).

### Example 9—Synthesis of 4-((S)-2-((S)-2-(4(6-((diethoxyphosphoryl)methyl)-1,2,4,5-tetrazin-3-yl)benzamido)-3-methylbutanamido)propanamido)benzyl (4-nitrophenyl) carbonate (9)

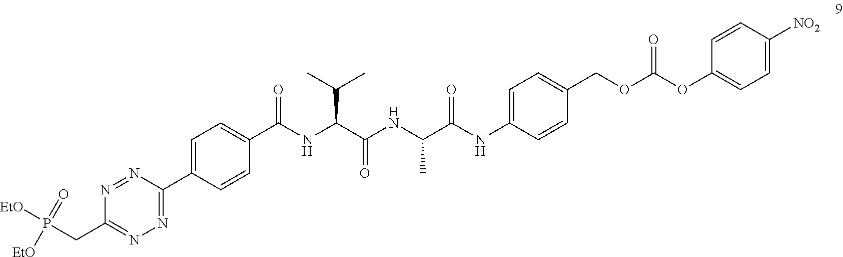 [SMILES: CCOP(=O)(Cc1nnc(-c2ccc(C(=O)N[C@H](C(=O)N[C@@H](C)C(=O)Nc3ccc(COC(=O)Oc4ccc([N+](=O)[O-])cc4)cc3)C(C)C)cc2)nn1)OCC]

To a solution of 8 (131 mg, 209 μmol, 1.00 eq) in dichloromethane (2.1 mL) was added PNP chloroformate (63.1 mg, 313 μmol, 1.50 eq) and DIPEA (54.5 μL, 313 μmol, 1.50 eq) and the mixture was stirred at room temperature After 3 h 20 mg of PNP chloroformate were added and the mixture stirred for another 1 h. It was diluted with dichloromethane and washed with saturated NaHCO_{3aq}. The organic phase was dried and the solvent evaporated under reduced pressure.

Purification via flash chromatography (dichloromethane/MeOH 20:1→10:1) yielded 9 as pink solid (101 mg, 61%).

### Example 10—Synthesis of 4-((S)-2-((S)-2-(4(6-((diethoxyphosphoryl)methyl)-1,2,4,5-tetrazin-3-yl)benzamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)(methyl)carbamate (10)

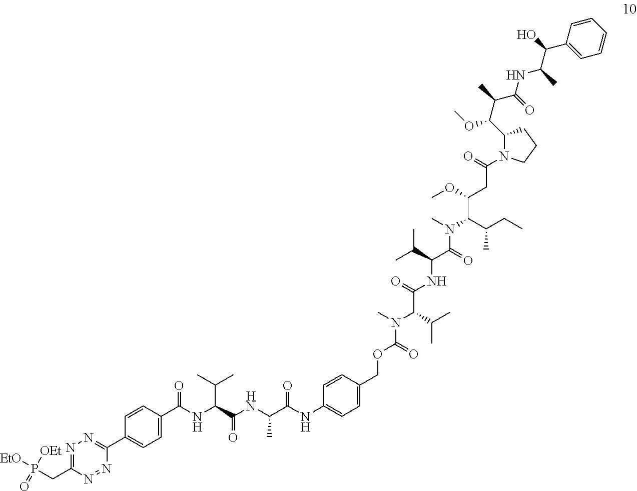 [SMILES: CCOP(=O)(Cc1nnc(-c2ccc(C(=O)N[C@H](C(=O)N[C@@H](C)C(=O)Nc3ccc(COC(=O)N(C)[C@H](C(=O)N[C@H](C(=O)N(C)[C@@H]([C@@H](C)CC)[C@@H](CC(=O)N4CCC[C@H]4[C@H](OC)[C@@H](C)C(=O)N[C@H](C)[C@@H](O)c4ccccc4)OC)C(C)C)C(C)C)cc3)C(C)C)cc2)nn1)OCC]

To a solution of 9 (20.0 mg, 25.2 μmol, 1.00 eq) and MMAE (18.1 mg, 25.2 μmol, 1.00 eq) in DMF (0.2 mL) was added pyridine (0.1 mL), HOBt monohydrate (0.773 mg, 5.05 μmol, 0.200 eq) and DIPEA (4.39 μL, 25.2 μmol, 1.00 eq) and the mixture stirred at room temperature over night. It was diluted with dichloromethane and washed with H₂O. The organic phase was dried and the solvent evaporated under reduced pressure.

Crude product 10 (34.0 mg, 98%) was directly used for the next step.

### Example 11—Synthesis of ((6-(4-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxopropan-2-yl)amino)-3-methyl-1-oxobutan-2-yl)carbamoyl)phenyl)-1,2,4,5-tetrazin-3-yl)methyl)phosphonic Acid (11)

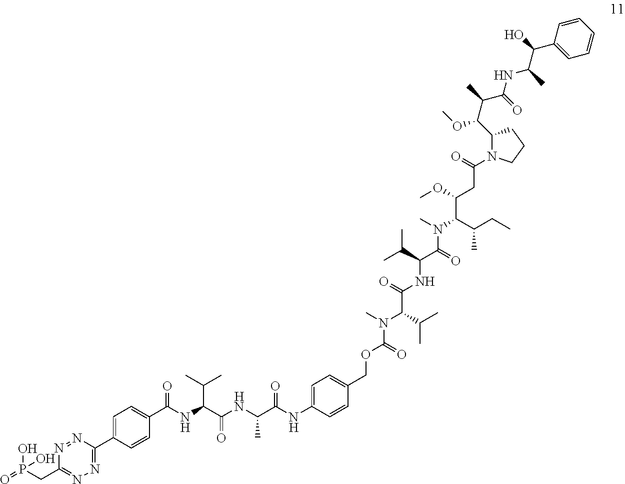 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)c2ccc(-c3nnc(CP(=O)(O)O)nn3)cc2)C(C)C)cc1)C(C)C]

To a solution of crude 10 (34.0 mg, 24.8 μmol, 1.00 eq) in DMF (0.5 mL) was added trimethylsilyl bromide (32.7 μL, 248 μmol, 10.0 eq) and the mixture stirred at room temperature over night. It was diluted with MeOH and H₂O and the mixture directly subjected to purification via HPLC.

Purification via HPLC yielded 11 (2.1 mg, 6%) as pink solid.

### Example 12—Synthesis of ((6-(4-(((2,5-dioxopyrrolidin-1-yl)oxy)carbonyl)phenyl)-1,2,4,5-tetrazin-3-yl)methyl)phosphonic Acid (12)

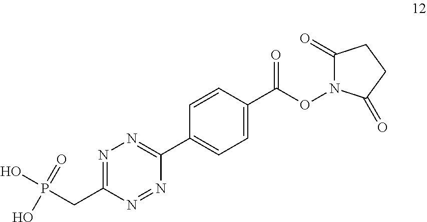 [SMILES: O=C(ON1C(=O)CCC1=O)c1ccc(-c2nnc(CP(=O)(O)O)nn2)cc1]

To a solution of 6 (30.8 mg, 68.5 μmol, 1.00 eq) in dichloromethane (0.7 mL) was added trimethylsilyl bromide (27.1 μL, 206 μmol, 3.00 eq) at 0° C. and the mixture stirred at this temperature for 3 h. DMF was added (0.7 mL) and the mixture stirred at room temperature for 3 d. It was diluted with MeOH and H₂O and the solvents evaporated under reduced pressure.

Purification via HPLC yielded 12 as pink powder (17.4 mg, 65%).

### Example 13—Synthesis of ((6-(4-(((2S)-1-(((2S)-1-(4-(((((2S)-1-(((14S,32S,33R,2S,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl)oxy)-1-oxopropan-2-yl)methyl)carbamoyl)oxy)methyl)phenyl)amino)-1-oxopropan-2-yl)amino)-3-methyl-1-oxobutan-2-yl)carbamoyl)phenyl)-1,2,4,5-tetrazin-3-yl)methyl)phosphonic Acid (13)

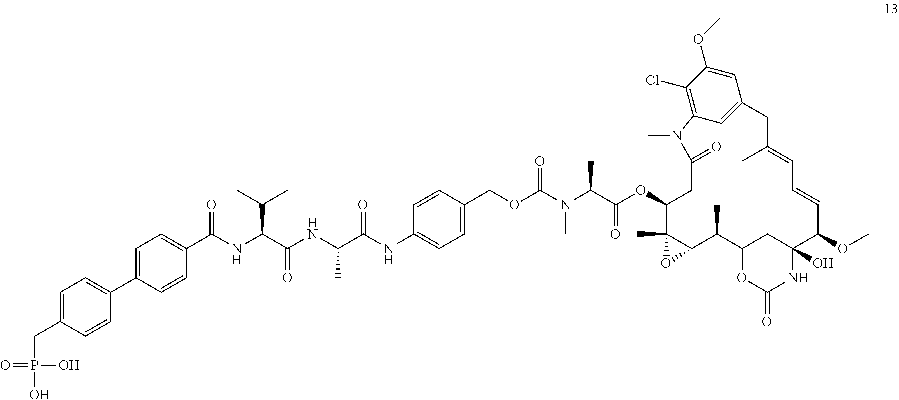 [SMILES: COc1cc2cc(c1Cl)N(C)C(=O)C[C@H](OC(=O)[C@H](C)N(C)C(=O)OCc1ccc(NC(=O)[C@H](C)NC(=O)[C@@H](NC(=O)c3ccc(-c4ccc(CP(=O)(O)O)cc4)cc3)C(C)C)cc1)[C@@]1(C)O[C@H]1[C@@H](C)C1C[C@@](O)(NC(=O)O1)[C@H](OC)/C=C/C=C(\C)C2]

To a solution of H-Val-Ala-PAB-Maytansinoid 28 (12.5 mg, 12.9 μmol, 1.00 eq) and 12 (11.2 mg, 28.4 μmol, 2.20 eq) in DMF (0.5 mL) was added NEt₃ (16.1 μL, 116 μmol, 9.00 eq) and the mixture stirred at room temperature over night. It was diluted with HCl, and the mixture directly subjected to purification via HPLC.

Purification via HPLC yielded 13 (7.8 mg, 48%) as pink powder.

### Example 14—Synthesis of 5-(6-((diethoxyphosphoryl)methyl)-1,2,4,5-tetrazin-3-yl)isophthalic Acid (14)

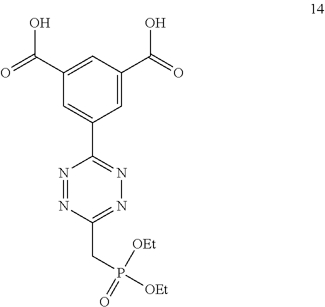 [SMILES: CCOP(=O)(Cc1nnc(-c2cc(C(=O)O)cc(C(=O)O)c2)nn1)OCC]

To a solution of 5-cyanoisophthalic acid (287 mg, 1.50 mmol, 1.00 eq) and diethyl cyanomethyl-phosphonate (972 μL, 6.00 mmol, 4.00 eq) in EtOH (1.5 mL) was added 3-mercaptopropionic acid (131 μL, 1.50 mmol, 1.00 eq) followed by hydrazine monohydrate (1.17 mL, 24.0 mmol, 16.0 eq) at 0° C. The mixture was stirred at room temperature over night. Afterwards NaNO₂ (2.07 g, 30.0 mmol, 20.0 eq) in H₂O was added and it was acidified to pH˜1 via dropwise addition of 1 M HCl_{aq}. The pink precipitate was filtered and washed with 0.1 M HCl_{aq}.

The crude product 14 (594 mg) was directly used for the next step without further purification.

### Example 15—Synthesis of bis(2,5-dioxopyrrolidin-1-yl) 5-(6-((diethoxyphosphoryl)methyl)-1,2,4,5-tetrazin-3-yl)isophthalate (15)

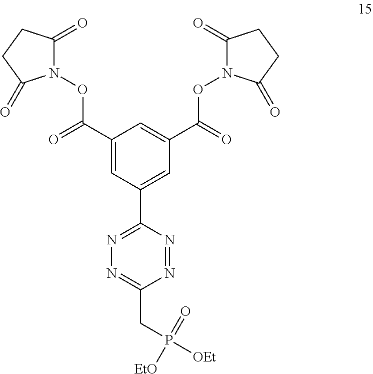 [SMILES: CCOP(=O)(Cc1nnc(-c2cc(C(=O)ON3C(=O)CCC3=O)cc(C(=O)ON3C(=O)CCC3=O)c2)nn1)OCC]

To a mixture of 14 (594 mg, 1.50 mmol, 1.00 eq) in dichloromethane (15 mL) was added N-hydroxysuccinimide (691 mg, 6.00 mmol, 4.00 eq) and EDC hydrochloride (1.15 g, 6.00 mmol, 4.00 eq) and the mixture was stirred at r.t.

Purification via flash chromatography (dichloromethane/MeOH 20:1) yielded 15 as pink solid (750 mg, 85% over 2 steps).

### Example 16—Synthesis of 1,1′-(5-(6-((diethoxyphosphoryl)methyl)-1,2,4,5-tetrazin-3-yl)-1,3-phenylene)bis(1-oxo-5,8,11,14,17,20,23,26-octaoxa-2-azanonacosan-29-oic Acid) (16)

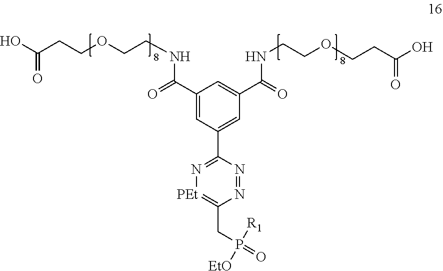

To a mixture of 15 (300 mg, 508 μmol, 1.00 eq) in DMF (5 mL) was added NEt₃ (140, μL, 103 mg, 1.02 mmol, 2.00 eq) and 1-amino-3,6,9,12,15,18,21,24-octaoxaheptacosan-27-oic acid (449 mg, 1.02 mmol, 2.00 eq) and the mixture was stirred at r.t. It was diluted with H₂O and the mixture directly subjected to purification via HPLC.

Purification via HPLC yielded 16 as pink oil (83 mg, 13%).

### Example 17—Synthesis of 2-(6-((diethoxyphosphoryl)methyl)-1,2,4,5-tetrazin-3-yl)acetic Acid (17)

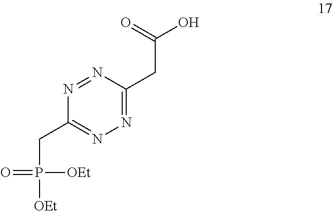 [SMILES: CCOP(=O)(Cc1nnc(CC(=O)O)nn1)OCC]

To a solution of cyanoacetic acid (3.40 g, 40.0 mmol, 4.00 eq) and diethyl cyanomethyl-phosphonate (1.62 mL, 10.0 mmol, 1.00 eq) in EtOH (10 mL) was added 3-mercaptopropionic acid (871 μL, 10.0 mmol, 1.00 eq) followed by hydrazine monohydrate (7.76 mL, 160 mmol, 16.0 eq) at 0° C. The mixture was stirred at room temperature over night. Afterwards NaNO₂ (13.8 g, 200 mmol, 20.0 eq) in H₂O was added and it was acidified to pH˜1 via dropwise addition of 1 M HCl_{aq}. It was extracted with EtOAc, the organic phase dries and the solvent evaporated under reduced pressure.

Purification via two times flash chromatography (dichloromethane/MeOH 50:1-10:1 and 20:1→10:1) yielded 17 as pink oil (260 mg, 9%).

### Example 18—Synthesis of diethyl ((6-(5-aminopyridin-2-yl)-1,2,4,5-tetrazin-3-yl)methyl)phosphonate (18)

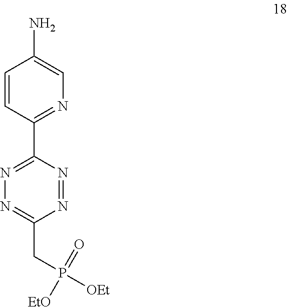 [SMILES: CCOP(=O)(Cc1nnc(-c2ccc(N)cn2)nn1)OCC]

To a solution of 5-amino-2-pyridinecarbonitrile (1.20 g, 10.1 mmol, 1.00 eq) and diethyl cyanomethyl-phosphonate (6.52 mL, 7.14 g, 40.3 mmol, 4.00 eq) in EtOH (10 mL) was added 3-mercaptopropionic acid (878 μL, 1.07 g, 10.1 mmol, 1.00 eq) followed by hydrazine monohydrate (7.82 mL, 8.07 g, 161 mmol, 16.0 eq) at 0° C. The mixture was stirred at room temperature over night.

The solvents were removed via rotary evaporation and the residue was purified via reverse phase flash chromatography. Oxidations of fractions containing dihydrotetrazine intermediate under air over night followed by evaporation of solvents yielded crude product 18.

Purification via flash chromatography (dichloromethane/MeOH 20:1) yielded 18 as dark red solid (555 mg, 17%).

### Example 19—Synthesis of ((6-(5-aminopyridin-2-yl)-1,2,4,5-tetrazin-3-yl)methyl)phosphonic Acid (19)

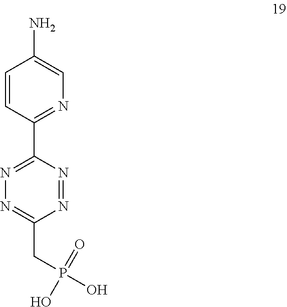 [SMILES: Nc1ccc(-c2nnc(CP(=O)(O)O)nn2)nc1]

To a solution of 18 (78.0 mg, 241 μmol, 1.00 eq) in dichloromethane/DMF (2:1, 3 mL) was added trimethylsilyl bromide (159 μL, 184 mg, 1.20 mmol, 5.00 eq) at 0° C. The mixture was stirred at room temperature over night. Afterwards it was diluted with MeOH and H₂O and the solvents evaporated.

Purification via HPLC yielded 19 as a red-pink powder (36.0 mg, 56%).

### Example 20—Synthesis of diethyl ((6-(4-(13-hydroxy-2,5,8,11-tetraoxatridecyl)phenyl)-1,2,4,5-tetrazin-3-yl)methyl)phosphonate (20)

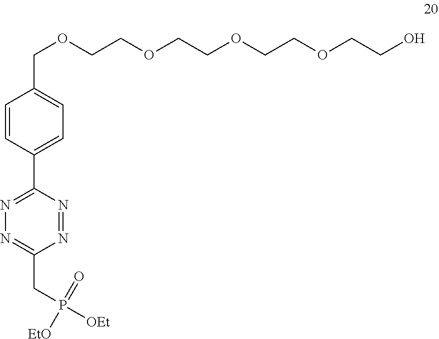 [SMILES: CCOP(=O)(Cc1nnc(-c2ccc(COCCOCCOCCOCCO)cc2)nn1)OCC]

A mixture of 4-(13-hydroxy-2,5,8,11-tetraoxatridecyl)benzonitrile (980 mg, 3.17 mmol, 5.00 eq), diethyl cyanomethyl-phosphonate (103 μL, 112 mg, 634 μmol, 1.00 eq), hydrazine monohydrate (1.54 mL, 1.59 g, 31.7 mmol, 50.0 eq) and Zn(OTf)₂ (11.5 mg, 31.7 μmol, 0.0500 eq) was stirred at 60° C. for 30 min. Afterwards NaNO₂ (875 mg, 12.7 mmol, 20.0 eq) in H₂O was added and it was acidified to pH˜3 via dropwise addition of 1 M HCl_{aq}. It was extracted with DCM and the solvent evaporated.

Purification via flash chromatography (dichloromethane/MeOH 40:1→10:1) yielded 20 as dark pink oil (9.0 mg, 3%).

### Example 21—Synthesis of ((6-(4-(13-hydroxy-2,5,8,11-tetraoxatridecyl)phenyl)-1,2,4,5-tetrazin-3-yl)methyl)phosphonic Acid (21)

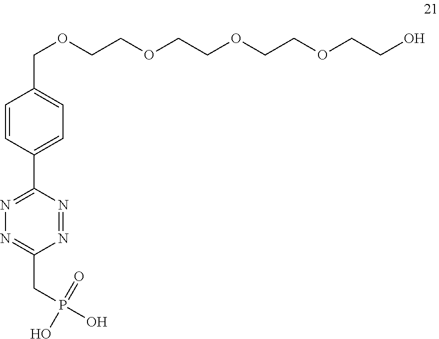 [SMILES: O=P(O)(O)Cc1nnc(-c2ccc(COCCOCCOCCOCCO)cc2)nn1]

To a solution of 20 (9.0 mg, 17.5 μmol, 1.00 eq) in DMF (0.2 mL) was added trimethylsilyl bromide (11.6 μL, 87.5 μmol, 5.00 eq) at 0° C. and the mixture stirred at room temperature for 90 min. The reaction was quenched with MeOH and H₂O and the mixture directly subjected to HPLC purification.

Purification via HPLC yielded 21 as dark pink oil (1.7 mg, 21%).

### Example 22—Synthesis of 4-(6-(2,5,8,11,14,17,20,23,26,29,32,35,38-tridecaoxanonatriacontyl)-1,2,4,5-tetrazin-3-yl)benzoic Acid (22)

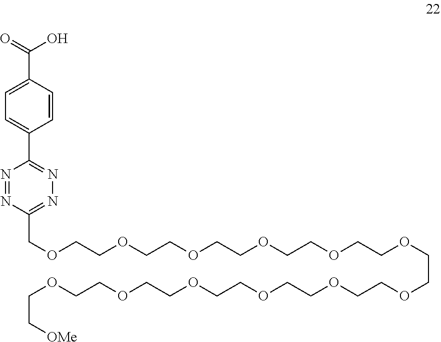 [SMILES: COCCOCCOCCOCCOCCOCCOCCOCCOCCOCCOCCOCCOCc1nnc(-c2ccc(C(=O)O)cc2)nn1]

To a mixture of 2,5,8,11,14,17,20,23,26,29,32,35,38-tridecaoxatetracontane-40-nitrile (1.20 g, 2.00 mmol, 1.00 eq), 4-cyanobenzoic acid (1.18 g, 8.00 mmol, 4.00 eq) in EtOH (2 mL) was added 3-mercaptopropionic acid (174 μL, 212 mg, 2.00 mmol, 1.00 eq) followed by hydrazine monohydrate (1.55 mL, 1.60 g, 32.0 mmol, 16.0 eq) at 0° C. The mixture was stirred at room temperature over night. Afterwards NaNO₂ (5.52 g, 80.0 mmol, 20.0 eq) in H₂O was added and it was acidified to pH˜3 via dropwise addition of 1 M HCl_{aq}. It was extracted with DCM and the solvents evaporated.

Purification via flash chromatography (dichloromethane/MeOH 20:1→10:1) yielded 22 as pink oil (352 mg, 23%).

### Example 23—Synthesis of 4-((S)-2-((S)-2-(4-(6-(2,5,8,11,14,17,20,23,26,29,32,35,38-tridecaoxanonatriacontyl)-1,2,4,5-tetrazin-3-yl)benzamido)-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate (23)

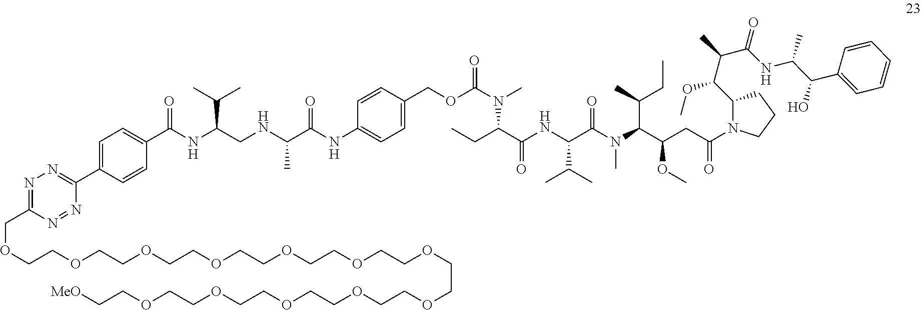 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](CC)N(C)C(=O)OCc1ccc(NC(=O)[C@H](C)NC[C@@H](NC(=O)c2ccc(-c3nnc(COCCOCCOCCOCCOCCOCCOCCOCCOCCOCCOCCOCCOC)nn3)cc2)C(C)C)cc1)C(C)C]

To a solution of H-Val-Ala-PAB-MMAE (27) (11.0 mg, 10.6 μmol, 1.00 eq) was added 22 (9.86 mg, 12.7 μmol, 1.20 eq), HATU (6.05 mg, 15.9 μmol, 1.50 eq) and DIPEA (4.62 μL, 3.42 mg, 26.5 μmol, 2.50 eq) and the mixture stirred at room temperature for 1 h. The crude reaction mixture was directly subjected to HPLC purification.

Purification via HPLC yielded 23 as pink powder (3.9 mg, 20%).

### Example 24—Synthesis of Ethyl hydrogen ((6-(4-(aminomethyl)phenyl)-1,2,4,5-tetrazin-3-yl)methyl)phosphonate (24)

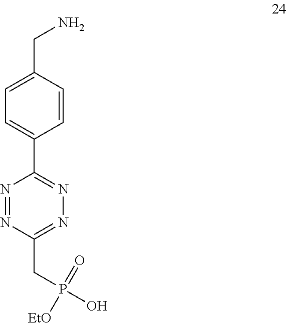 [SMILES: CCOP(=O)(O)Cc1nnc(-c2ccc(CN)cc2)nn1]

To a mixture of 4-aminomethyl-benzonitrile (169 mg, 1.00 mmol, 1.00 eq) and diethyl cyanomethyl-phosphonate (1.62 mL, 1.77 g, 10.0 mmol, 10.0 eq) was added hydrazine monohydrate (2.43 μm, 2.50 g, 50.0 mmol, 50.0 eq) and Zn(OTf)₂ (18.2 mg, 50.0 μmol, 0.0500 eq). The mixture was stirred at room temperature over night. Afterwards NaNO₂ (1.38 g, 20.0 mmol, 20.0 eq) in H₂O was added and it was acidified to pH˜3 via dropwise addition of 1 M HCl_{aq}.

Purification via HPLC yielded 24 as a pink powder (26.1 mg, 30%).

### Example 25—Synthesis of ((6-(4-(aminomethyl)phenyl)-1,2,4,5-tetrazin-3-yl)methyl)phosphonic Acid (25)

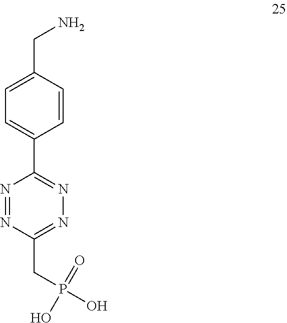 [SMILES: NCc1ccc(-c2nnc(CP(=O)(O)O)nn2)cc1]

To a solution of 24 (94.0 mg, 272 μmol, 1.00 eq) in DMF was added trimethylsilyl bromide (382 μL, 416 mg, 2.72 mmol, 10.0 eq) at 0° C. The mixture was stirred at room temperature for 5 h. Afterwards it was diluted with MeOH and H₂O and the solvents evaporated.

Purification via HPLC yielded 2 as a pink powder (26.1 mg, 30%).

### Example 26—Synthesis of 4-(6-(phosphonomethyl)-1,2,4,5-tetrazin-3-yl)benzoic Acid (26)

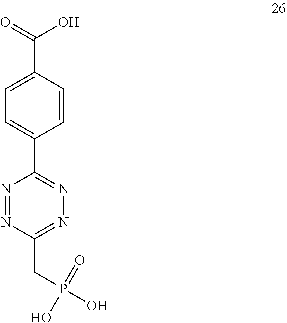 [SMILES: O=C(O)c1ccc(-c2nnc(CP(=O)(O)O)nn2)cc1]

To a solution of 4-cyanobenzoic acid (649 mg, 4.37 mmol, 1.00 eq) and diethyl cyanomethyl-phosphonate (3.15 mL, 3.45 g, 30.0 mmol, 6.86 eq) was added hydrazine monohydrate (7.28 mL, 7.51 g, 150 mmol, 34.3 eq) and Zn(OTf)₂ (54.5 mg, 150 μmol, 0.0500 eq). The mixture was stirred at room for 4 h. Afterwards NaNO₂ (5.52 g, 80.0 mmol, 20.0 eq) in H₂O was added and it was acidified to pH˜3 via dropwise addition of 1 M HCl_{aq}. The pink precipitate was filtered and washed with 0.1 M HCl, to provide a crude mixture of 3 and 4-(6-((ethoxy(hydroxy)phosphoryl)methyl)-1,2,4,5-tetrazin-3-yl)benzoic acid.

To a solution of the crude precipitate in DMF was added trimethylsilyl bromide (2.64 mL, 3.06 g, 20.0 mmol, 5.00 eq) at 0° C. The mixture was stirred at room temperature for 5 h. Afterwards it was diluted with MeOH and H₂O and the solvents evaporated.

Purification via HPLC yielded 26 as a pink powder (64.5 mg, 5%).

### Example 27—Synthesis of H-Val-Ala-PAB-MMAE (4-((S)-2-((S)-2-amino-3-methylbutanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate) (27)

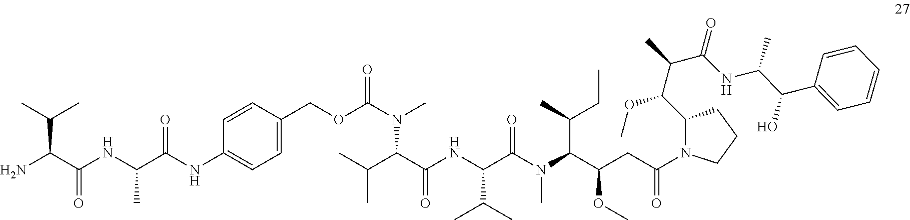 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](N)C(C)C)cc1)C(C)C]

To a solution of Fmoc-Val-Ala-PAB-PNP (34.1 mg, 50.1 μmol, 1.00 eq) and MMAE (38.0 mg, 50.1 μmol, 1.00 eq) in DMF (0.5 mL) was added pyridine (0.25 mL), HOBt (0.768 mg, 5.01 μmol, 0.100 eq) and DIPEA (8.73 NL, 6.48 mg, 50.1 μmol, 1.00 eq) and the mixture stirred 8 h at room temperature. After complete conversion piperidine (0.125 mL) was added and the mixture stirred for another 5 min and afterwards directly subjected to HPLC purification.

Purification via HPLC yielded 27 (44.3 mg, 85%) as white solid.

### Example 28—Synthesis of H-Val-Ala-PAB-Maytansinoid ((14S,32S,33R,2S,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl N-(((4-((S)-2-((S)-2-amino-3-methylbutanamido)propanamido)benzyl)oxy)carbonyl)-N-methyl-L-alaninate) (28)

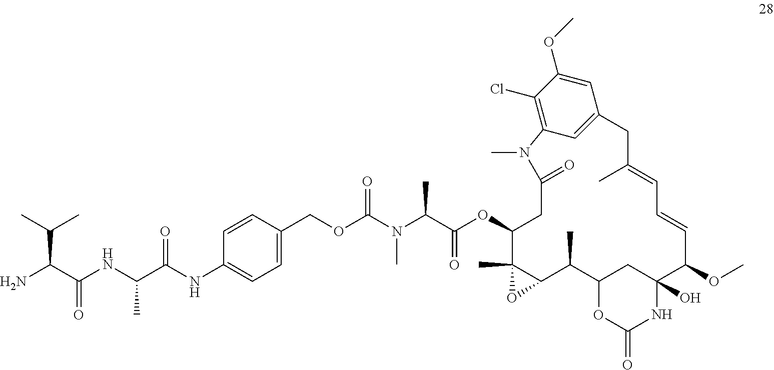 [SMILES: COc1cc2cc(c1Cl)N(C)C(=O)C[C@H](OC(=O)[C@H](C)N(C)C(=O)OCc1ccc(NC(=O)[C@H](C)NC(=O)[C@@H](N)C(C)C)cc1)[C@@]1(C)O[C@H]1[C@@H](C)C1C[C@@](O)(NC(=O)O1)[C@H](OC)/C=C/C=C(\C)C2]

To a solution of Fmoc-Val-Ala-PAB-PNP (41.0 mg, 63.0 μmol, 1.00 eq) and maytansinoid (42.9 mg, 63.0 μmol, 1.00 eq) in DMF (0.6 mL) was added pyridine (0.28 mL), HOBt (0.965 mg, 6.30 μmol, 0.100 eq) and DIPEA (10.9 μL, 8.14 mg, 63.0 μmol, 1.00 eq) and the mixture stirred 8 h at room temperature. After complete conversion, piperidine (0.15 mL) was added and the mixture stirred for another 5 min and afterwards directly subjected to HPLC purification.

Purification via HPLC yielded 28 (25.8 mg, 42%) as white solid.

### Example 29—Synthesis of diethyl (6-cyanopyridin-3-yl)phosphonate (29)

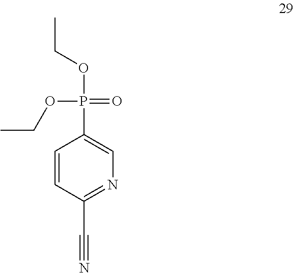 [SMILES: CCOP(=O)(OCC)c1ccc(C#N)nc1]

To a mixture of diethyl phosphite (567 μL, 608 mg, 4.40 mmol, 1.10 eq), NEt₃ (610 μL, 445 mg, 4.40 mmol, 1.10 eq) and Pd(PPh₃)₄(231 mg, 200 μmol, 0.0500 eq) was added 5-bromo-2-cyanopyridine (732 mg, 4.00 mmol, 1.00 eq) and 1 mL of toluene and the mixture stirred at 90° C. for 1 h.

Purification via flash chromatography (cyclohexane/EtOAc 1:1) yielded 29 as colorless oil (650 mg, 68%).

### Example 30—Synthesis of 3-(6-(4-(hydroxy(methoxy)phosphoryl)phenyl)-1,2,4,5-tetrazin-3-yl)propanoic Acid (30)

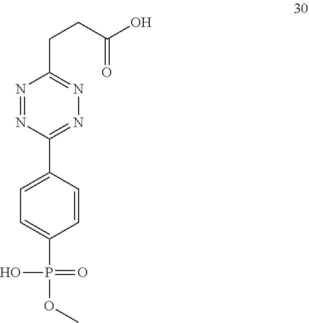 [SMILES: COP(=O)(O)c1ccc(-c2nnc(CCC(=O)O)nn2)cc1]

To a solution of dimethyl (4-cyanophenyl)phosphonate (54.0 mg, 256 μmol, 1.00 eq) and 3-cyanopropionic acid (87.0 mg, 878 μmol, 3.43 eq) in EtOH (0.26 mL) was added hydrazine monohydrate (170 μL, 176 mg, 3.51 mmol, 13.7 eq) followed by 3-mercaptopropionic acid (19.1 μL, 23.3 mg, 220 μmol, 0.859 eq). The mixture was stirred at room temperature overnight. Afterwards sodium nitrite (303 mg, 4.39 mmol, 20.0 eq) was added and the solution was acidified via dropwise addition of 1 M HCl_{aq}. It was extracted with EtOAc (3×) and the combined organic phases dried over sodium sulfate and evaporated under reduced pressure.

Purification via reverse phase flash chromatography (0-50% MeOH in H₂O) yielded 30 as a pink solid (40.4 mg, 49%).

### Example 31—Synthesis of 3-(6-(4-phosphonophenyl)-1,2,4,5-tetrazin-3-yl)propanoic Acid (31)

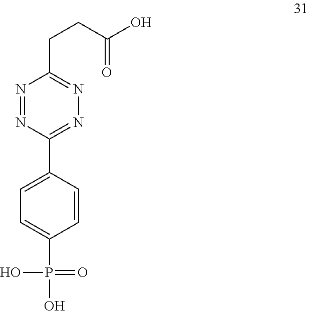 [SMILES: O=C(O)CCc1nnc(-c2ccc(P(=O)(O)O)cc2)nn1]

To a solution of 30 (15.4 mg, 47.5 μmol, 1.00 eq) in DMF (0.5 mL) was added trimethylsilyl bromide (31.3 μL, 36.4 mg, 237 μmol, 5.00 eq) at 0° C. The mixture was stirred at room temperature for 30 min. Water was added and the mixture directly subjected to purification.

Purification via reverse phase flash chromatography (0-50% MeOH in H₂O) yielded 31 as a pink solid (12.3 mg, 83%).

### Example 32—Synthesis of (4-(6-(3-((2,5-dioxopyrrolidin-1-yl)oxy)-3-oxopropyl)-1,2,4,5-tetrazin-3-yl)phenyl)phosphonic Acid (32)

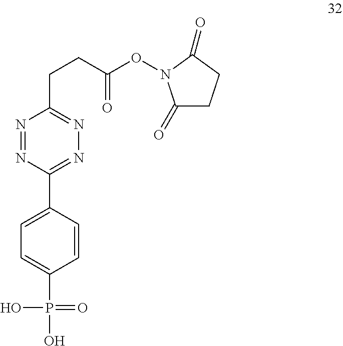 [SMILES: O=C(CCc1nnc(-c2ccc(P(=O)(O)O)cc2)nn1)ON1C(=O)CCC1=O]

To a solution of 31 (9.50 mg, 30.6 μmol, 1.00 eq) in DMF (0.3 mL) was added NEt₃ (5.09 μL, 3.72 mg, 36.8 μmol, 1.20 eq) and N,N,N′,N′-Tetramethyl-O—(N-succininidyl)uroniumtetrafluorborat (TSTU) (11.1 mg, 36.8 μmol, 1.20 eq). The mixture was stirred at room temperature. After 1.5 h another 10 mg of TSTU and 4 μL of NEtₐ was added and the mixture stirred for another 30 min. It was diluted with water and the mixture directly subjected to purification via HPLC.

Purification via HPLC yielded 32 as a pink solid (2.2 mg, 18%).

### Example 33—Synthesis of 2-(6-(phosphonomethyl)-1,2,4,5-tetrazin-3-yl)acetic Acid (33)

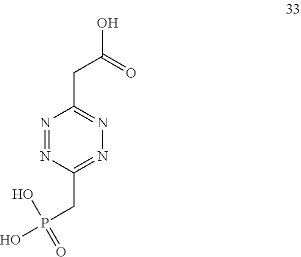 [SMILES: O=C(O)Cc1nnc(CP(=O)(O)O)nn1]

To a solution of 17 (30.0 mg, 103 μmol, 1.00 eq) in DMF (0.5 mL) was added trimethylsilyl bromide (136 μL, 158 mg, 1.03 mmol, 10.0 eq) at 0° C. The mixture was stirred at room temperature for 3.5 h. Water was added and the mixture directly subjected to purification via HPLC.

Purification via HPLC yielded 33 as a pink solid (2.8 mg, 12%).

### Example 34—Synthesis of diethyl (4-(6-(5-aminopyrimidin-2-yl)-1,2,4,5-tetrazin-3-yl)phenyl)phosphonate (34)

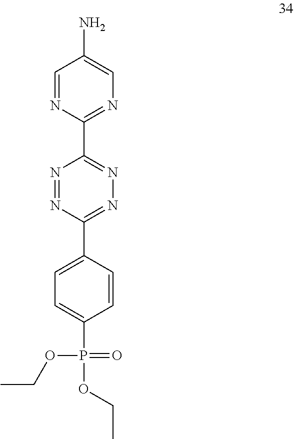 [SMILES: CCOP(=O)(OCC)c1ccc(-c2nnc(-c3ncc(N)cn3)nn2)cc1]

To a solution of diethyl (4-cyanophenyl)phosphonate (398 mg, 256 μmol, 2.00 eq) and 2-cyano-5-aminopyrimidine (100 mg, 833 μmol, 1.00 eq) in EtOH (0.8 mL) was added hydrazine monohydrate (646 μL, 667 mg, 13.3 mmol, 16.0 eq) followed by 3-mercaptopropionic acid (218 μL, 265 mg, 2.50 mmol, 3.00 eq). The mixture was stirred at room temperature overnight. The reaction was diluted with water and extracted with dichloromethane (2×). The combined organic phases were dried over sodium sulfate and evaporated under reduced pressure.

The residue was dissolved in 5 mL of dichloromethane and 150 mg p-benzoquinone was added, the mixture stirred for 5 min and directly subjected to column chromatography.

Purification via flash chromatography (dichloromethane/MeOH following a gradient from 20:1 to 10:1) yielded 34 as an orange-brown oil (139 mg, 43%).

### Example 35—Synthesis of (4-(6-(5-aminopyrimidin-2-yl)-1,2,4,5-tetrazin-3-yl)phenyl)phosphonic Acid (35)

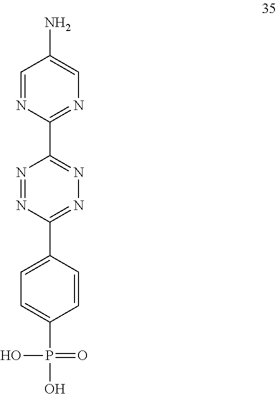 [SMILES: Nc1cnc(-c2nnc(-c3ccc(P(=O)(O)O)cc3)nn2)nc1]

To a solution of 34 (22.8 mg, 58.9 μmol, 1.00 eq) in DMF (0.6 mL) was added trimethylsilyl bromide (155 μL, 180 mg, 1.18 mmol, 20.0 eq) at 0° C. The mixture was stirred at room temperature for 2 h. Water was added and the mixture stirred for 5 min. The formed precipitate was filtered off and washed with water and acetone.

35 was isolated as a red solid (2.8 mg, 12%).

### Example 36—Synthesis of diethyl (6-(6-(5-aminopyridin-2-yl)-1,2,4,5-tetrazin-3-yl)pyridin-3-yl)phosphonate (36)

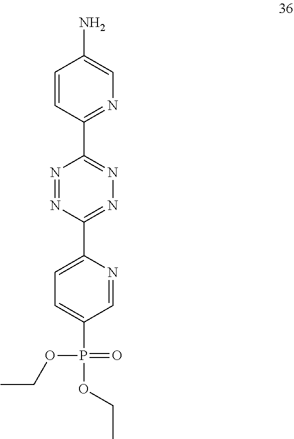 [SMILES: CCOP(=O)(OCC)c1ccc(-c2nnc(-c3ccc(N)cn3)nn2)nc1]

To a solution of 29 (240 mg, 999 μmol, 1.00 eq) and 2-cyano-5-aminopyridine (476 mg, 4.00 mmol, 4.00 eq) in EtOH (1 mL) was added hydrazine monohydrate (775 μL, 800 mg, 13.3 mmol, 16.0 eq) followed by 3-mercaptopropionic acid (87.1 μL, 106 mg, 999 μmol, 1.00 eq). The mixture was stirred at room temperature overnight. Then 1 mL of DMF was added to dissolve formed precipitate and the mixture was stirred at 60° C. for 4 h. The mixture was directly subjected to purification via reverse phase column chromatography.

The isolated dihydrotetrazine compound (9.9 mg) was dissolved in 0.5 mL DMF/MeOH, 2.7 mg p-benzoquinone was added and the mixture stirred for 5 min. The mixture was directly subjected to purification via HPLC.

Purification via HPLC yielded 36 as an orange solid (7.3 mg, 2%).

### Example 37—Synthesis of (6-(6-(5-aminopyridin-2-yl)-1,2,4,5-tetrazin-3-yl)pyridin-3-yl)phosphonic Acid (37)

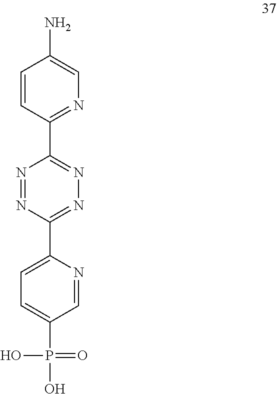 [SMILES: Nc1ccc(-c2nnc(-c3ccc(P(=O)(O)O)cn3)nn2)nc1]

To a solution of 36 (3.8 mg, 9.81 μmol, 1.00 eq) in DMF (0.1 mL) was added trimethylsilyl bromide (12.9 μL, 15.0 mg, 98.1 μmol, 10.0 eq) at 0° C. The mixture was stirred at room temperature overnight. Afterwards 12 μL of TMS-Br were added and the mixture stirred for 5 h at rt. Afterwards 20 μL of TMS-Br were added and the mixture stirred for 6 h at rt. Water was added and the mixture directly subjected to purification via HPLC.

Purification via HPLC yielded 37 as an orange solid (1.8 mg, 55%).

### Example 38—Synthesis of (6-(6-((diethoxyphosphoryl)methyl)-1,2,4,5-tetrazin-3-yl)pyridin-3-yl)glycine (38)

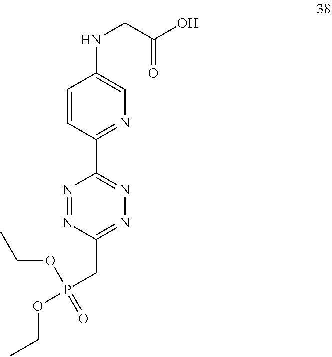 [SMILES: CCOP(=O)(Cc1nnc(-c2ccc(NCC(=O)O)cn2)nn1)OCC]

To a solution of (6-cyanopyridin-3-yl)glycine (340 mg, 1.92 mmol, 1.00 eq) and diethyl cyanomethylphosphonate (621 μL, 680 mg, 3.84 mmol, 2.00 eq) in EtOH (2 mL) was added hydrazine monohydrate (1.49 mL, 1.54 g, 30.7 mmol, 16.0 eq) followed by 3-mercaptopropionic acid (502 μL, 611 mg, 5.76 mmol, 3.00 eq). The mixture was stirred at room temperature overnight. Afterwards volatile components were removed under reduced pressure and the residue purified via reverse phase column chromatography.

The isolated dihydrotetrazine compound (320 mg) was dissolved in 10 mL DMF/MeOH, 108 mg p-benzoquinone was added and the mixture stirred for 5 min.

The mixture was directly subjected to purification via column chromatography to yield 35 mg of product 38. Additionally isolated mixed fractions were subjected to purification via HPLC.

Purification via HPLC yielded 38 as a red solid (130 mg, 18%).

### Example 39—Synthesis of (6-(6-(phosphonomethyl)-1,2,4,5-tetrazin-3-yl)pyridin-3-yl)glycine (39)

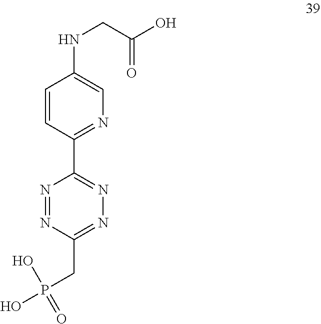 [SMILES: O=C(O)CNc1ccc(-c2nnc(CP(=O)(O)O)nn2)nc1]

To a solution of 38 (35.0 mg, 91.5 μmol, 1.00 eq) in DMF (1 mL) was added trimethylsilyl bromide (109 μL, 126 mg, 824 μmol, 9.00 eq) at 0° C. The mixture was stirred at room temperature for 4 h. Water was added and the mixture stirred for 30 min. It was extracted with EtOAc and the aqueous phase lyophilized. The resulting oil was precipitated from acetone and filtered off. 39 was isolated as a red solid (30.0 mg, 90%).

### Example 40—Synthesis of Boc-Val-Cit-PAB-MMAE 4-((S)-2-((S)-2-((tert-butoxycarbonyl)amino)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)(methyl)carbamate (40)

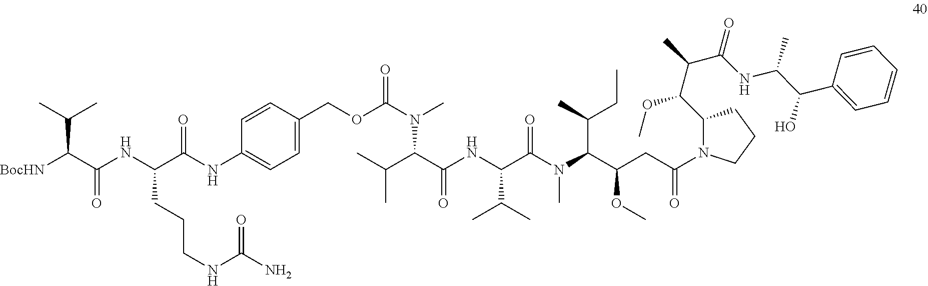 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OC(C)(C)C)C(C)C)cc1)C(C)C]

To a solution of Boc-Val-Cit-PAB-PNP (18.0 mg, 27.9 μmol, 1.00 eq) and MMAE (20.0 mg, 27.9 μmol, 1.00 eq) in DMF (0.25 mL) was added pyridine (0.125 mL), HOBt (0.428 mg, 2.79 μmol, 0.100 eq) and DIPEA (4.86 μL, 3.61 mg, 27.9 μmol, 1.00 eq) and the mixture stirred 2 d at room temperature. Afterwards the mixture was directly subjected to HPLC purification.

Purification via HPLC yielded 40 (25.2 mg, 74%) as white solid.

### Example 41—Synthesis of H-Val-Cit-PAB-MMAE 4-((S)-2-((S)-2-amino-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate (41)

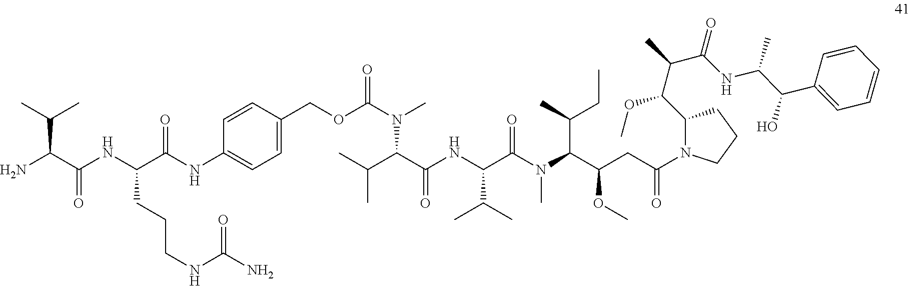 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of 40 (25.2 mg, 20.6 μmol, 1.00 eq) in dichloromethane (0.8 mL) was added trifluoroacetic acid (0.2 mL) and the mixture stirred 15 min at room temperature. Afterwards the solvents were removed under reduced pressure.

Purification via HPLC yielded 41 (17.0 mg, 73%) as white solid.

### Example 42—Synthesis of 4-((S)-2-((S)-2-(2-((6-(6-((diethoxyphosphoryl)methyl)-1,2,4,5-tetrazin-3-yl)pyridin-3-yl)amino)acetamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate (42)

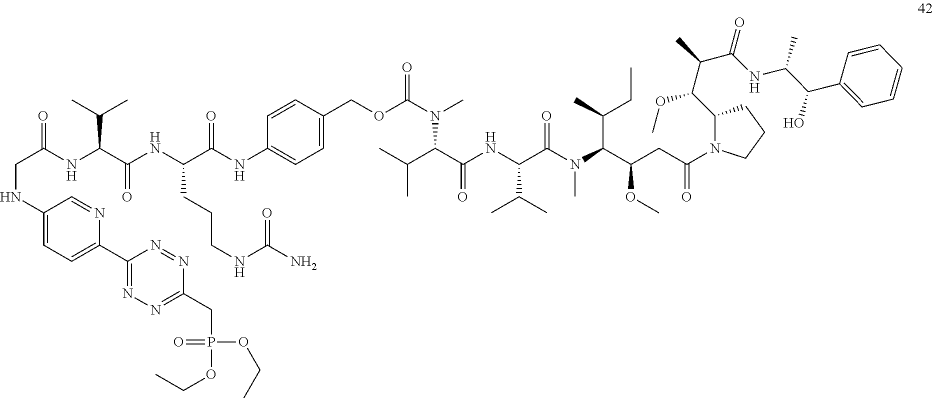 [SMILES: CCOP(=O)(Cc1nnc(-c2ccc(NCC(=O)N[C@H](C(=O)N[C@@H](CCCNC(N)=O)C(=O)Nc3ccc(COC(=O)N(C)[C@H](C(=O)N[C@H](C(=O)N(C)[C@@H]([C@@H](C)CC)[C@@H](CC(=O)N4CCC[C@H]4[C@H](OC)[C@@H](C)C(=O)N[C@H](C)[C@@H](O)c4ccccc4)OC)C(C)C)C(C)C)cc3)C(C)C)cn2)nn1)OCC]

To a solution of 41 (17.0 mg, 15.1 μmol, 1.00 eq) in DMF (0.3 mL) was added 38 (5.79 mg, 15.1 μmol, 1.00 eq), EDC (3.48 mg, 18.2 μmol, 1.20 eq) and NEt₃ (3.15 μL, 2.30 mg, 22.7 μmol, 1.50 eq) and the mixture stirred for 4 h at room temperature. Afterwards additional 10 mg of 38, 8 mg EDC and 8 μL NEt3 were added and the mixture stirred for another 4 h. The mixture was directly subjected to HPLC purification.

Purification via HPLC yielded 42 (2.4 mg, 11%) as light red solid.

### Example 43—Synthesis of ((6-(5-((2-(((S)-1-(((S)-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-2-oxoethyl)amino)pyridin-2-yl)-1,2,4,5-tetrazin-3-yl)methyl)phosphonic Acid (43)

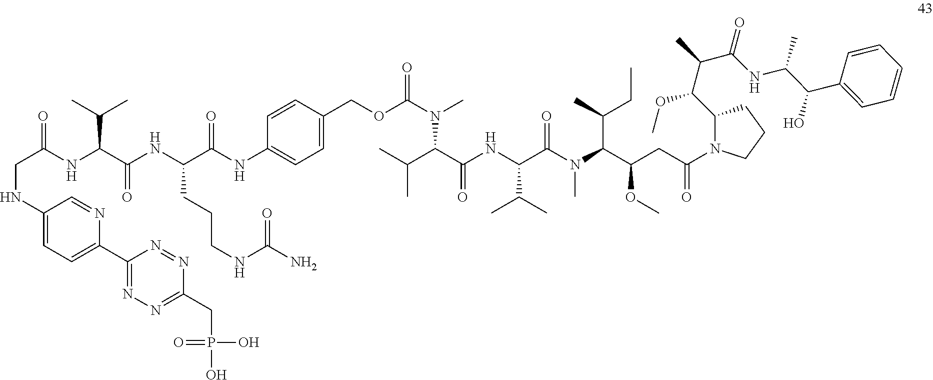 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CNc2ccc(-c3nnc(CP(=O)(O)O)nn3)nc2)C(C)C)cc1)C(C)C]

To a solution of 42 (2.4 mg, 1.61 μmol, 1.00 eq) in DMF (0.1 mL) was added TMS-Br (10.6 μL, 12.3 mg, 80.7 μmol, 50.0 eq) and the mixture stirred for 2 d at room temperature. Water was added and the mixture extracted with EtOAc. The aqueous phase was lyophilized.

43 was isolated as light red solid (1 mg, 43%).

### Example 44—Synthesis of (2S,3S,4S,5R,6S)-6-(2-(3-aminopropanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic Acid (44)

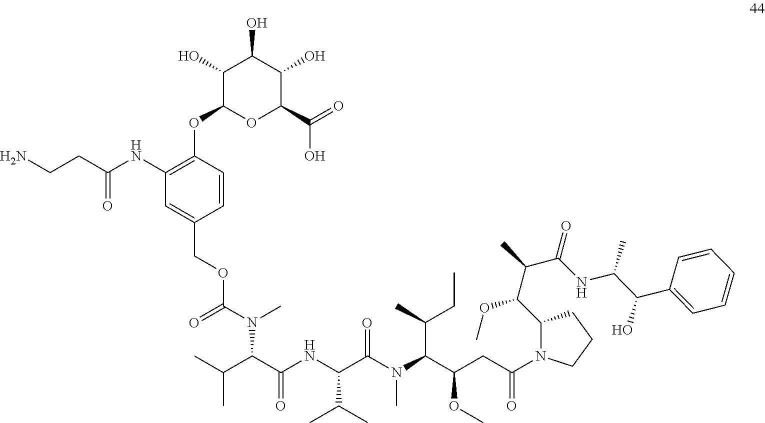 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCN)c1)C(C)C]

To a solution of MMAE (19.6 mg, 27.4 μmol, 1.00 eq) and A-D-glucuronide-PNP-carbonate (25.0 mg, 27.4 μmol, 1.00 eq) in DMF (0.5 mL) was added pyridine (0.25 mL), HOBt (0.419 mg, 2.74 μmol, 0.100 eq) and DIPEA (4.77 μL, 27.4 μmol, 1.00 eq). The mixture was stirred at RT overnight. Afterwards 1 M NaOH_{aq} (0.274 mL, 274 μmol, 10.0 eq) was added and the mixture stirred for 1 h at RT. The reaction mixture was directly subjected to purification via HPLC.

Purification via HPLC yielded 44 (17.0 mg, 55%)

### Example 45—Synthesis of (2S,3S,4S,5R,6S)-6-(2-(3-aminopropanamido)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic Acid (45)

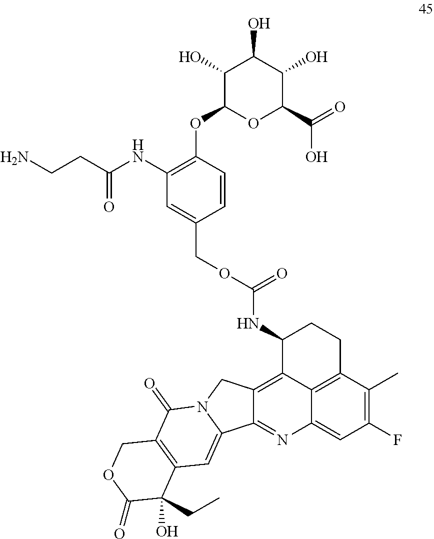 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCN)c1)CC3]

To a solution of exatecan mesylate (14.5 mg, 27.4 μmol, 1.00 eq) and A-D-glucuronide-PNP-carbonate (25.0 mg, 27.4 μmol, 1.00 eq) in DMF (0.5 mL) was added pyridine (0.25 mL), HOBt (0.419 mg, 2.74 μmol, 0.100 eq) and DIPEA (4.77 μL, 27.4 μmol, 1.00 eq). The mixture was stirred at RT overnight. Afterwards 1 M NaOH_{aq} (0.274 mL, 274 μmol, 10.0 eq) was added and the mixture stirred for 1 h at RT. The reaction mixture was directly subjected to purification via HPLC.

Purification via HPLC yielded 45 (3.0 mg, 13%)

### Example 46—Synthesis of 4-((S)-4-amino-2-((S)-2-((S)-2-aminopropanamido)propanamido)-4-oxobutanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl(methyl)amino)-3-methyl-1-oxobutan-2-yl)amino)-3-methyl-1-oxobutan-2-yl)(methyl)carbamate (46)

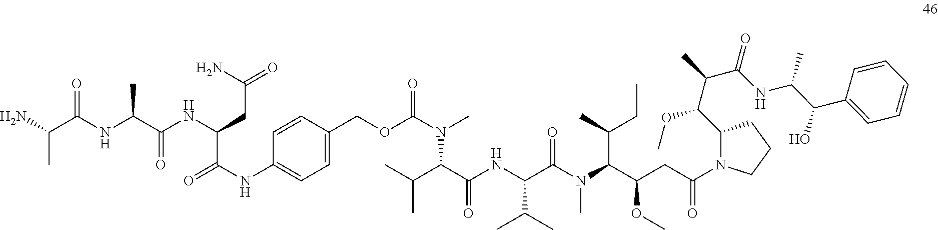 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(N)=O)NC(=O)[C@H](C)NC(=O)[C@H](C)N)cc1)C(C)C]

To a solution of MMAE (28.1 mg, 39.1 μmol, 1.00 eq) and Fmoc-Ala-Ala-Asn-PAB-PNP (30.0 mg, 39.1 μmol, 1.00 eq) in DMF (0.5 mL) was added pyridine (0.25 mL), HOBt (0.598 mg, 3.91 μmol, 0.100 eq) and DIPEA (6.81 μL, 39.1 μmol, 1.00 eq). The mixture was stirred at RT overnight. Afterwards 1 M NaOH_{aq} (0.391 mL, 391 μmol, 10.0 eq) was added and the mixture stirred for 1 h at RT. The reaction mixture was directly subjected to purification via HPLC.

Purification via HPLC yielded 46 (16.6 mg, 38%)

### Example 47—Synthesis of (3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2-oxa-15,16-dithia-5,8,11-triazanonadecane-19-oic Acid (47)

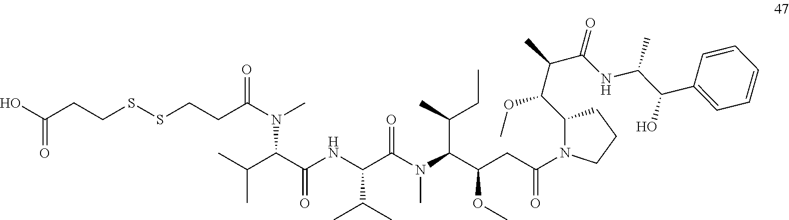 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCSSCCC(=O)O)C(C)C]

To a solution of 3,3′-dithiodipropionic Acid (29.3 mg, 139.3 μmol, 2.00 eq) and HATU (26.5 mg, 69.6 μmol, 1.00 eq) in DMF (0.5 mL) was added DIPEA (12.1 μL, 69.6 μmol, 1.00 eq) and the mixture stirred for 1 h at RT. Afterwards MMAE (50.0 mg, 69.6 μmol, 1.00 eq) and DIPEA (12.1 μL, 69.6 μmol, 1.00 eq) was added, the reaction mixture stirred at RT overnight and directly subjected to HPLC purification.

Purification via HPLC yielded 47 (43.0 mg, 68%)

### Example 48—Synthesis of tert-butyl (2-(6-((diethoxyphosphoryl)methyl)-1,2,4,5-tetrazin-3-yl)ethyl)carbamate (48)

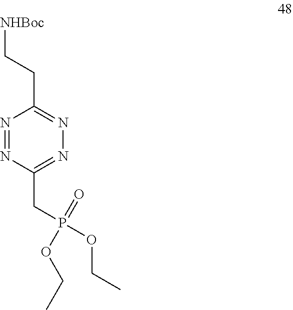 [SMILES: CCOP(=O)(Cc1nnc(CCNC(=O)OC(C)(C)C)nn1)OCC]

A solution of tert-butyl N-(2-cyanoethyl) carbamate (500 mg, 2.94 mmol, 1.00 eq), diethyl cyanomethylphosphonate (950 μL, 5.88 mmol, 2.00 eq) and 3-mercaptopropionic acid (768 μL, 8.813 mmol, 3.00 eq) in EtOH/DMF (1:1, 10 mL) was added to hydrazine monohydrate (2.28 mL, 47.0 mmol, 16.0 eq) and the mixture stirred at RT overnight Afterwards NaNO₂ (4.99 g, 58.8 mmol, 20.0 eq) in H₂O was added and it was acidified to pH-3 via dropwise addition of 1 M HCl_{aq}. The formed precipitate was filtered off and the aqueous phase was extracted with ethyl acetate. The combined organic layers were dried over Na₂SO₄ and evaporated under reduced pressure. Purification via reversed phase column chromatography yielded 48 (360 mg, 33%).

### Example 49—Synthesis of diethyl ((6-(2-aminoethyl)-1,2,4,5-tetrazin-3-yl)methyl)phosphonate (49)

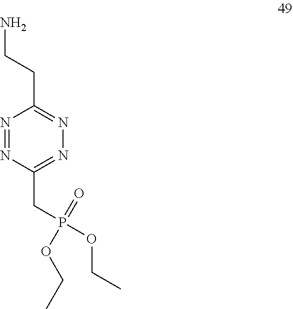 [SMILES: CCOP(=O)(Cc1nnc(CCN)nn1)OCC]

To a solution of 48 (160 mg, 0.426 mmol, 1.00 eq) in DCM (2 ml) was added TFA (328 μl, 4.26 mmol, 10.0 eq) and the mixture stirred at RT overnight. The solvents were removed under reduced pressure.

Evaporation yielded crude 49 (192 mg) which was used as crude product for subsequent reactions.

### Example 50—Synthesis of ((6-(2-aminoethyl)-1,2,4,5-tetrazin-3-yl)methyl)phosphonic Acid (50)

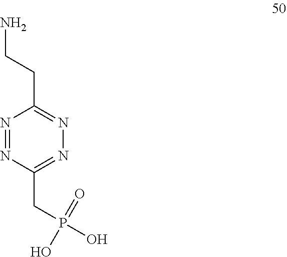 [SMILES: NCCc1nnc(CP(=O)(O)O)nn1]

To a solution of crude 49 (100 mg, 257 μmol, 1.00 eq) in DMF (5.1 mL) was added trimethylsilyl bromide (170 μL, 1.28 mmol, 5.00 eq) at 0° C. and the mixture stirred at room temperature overnight. Water was added and the solvents were evaporated under reduced pressure. Purification via reversed Phase chromatography (H₂O/MeOH, 0-50%, 10 g Sfaer C18)

Purification via reversed phase chromatography yielded 50 (12.5 mg, 22%)

### Example 51—Synthesis of diethyl ((6-(5-aminopyrimidin-2-yl)-1,4-dihydro-1,2,4,5-tetrazin-3-yl)methyl)phosphonate (51)

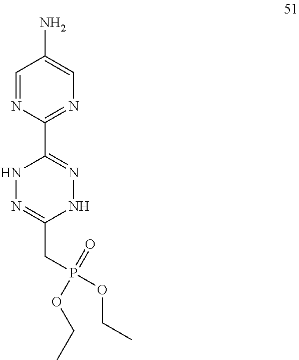 [SMILES: CCOP(=O)(CC1=NNC(c2ncc(N)cn2)=NN1)OCC]

A solution of 5-amino-2-pyrimidinecarbonitrile (200 mg, 1.67 mmol, 1.00 eq), diethyl cyanomethylphosphonate (0.539 mL, 3.33 mmol, 2.00 eq) and 3-mercaptopropionic acid (435 μL, 5.00 mmol, 3.00 eq) in EtOH/DMF (1:1, 5 mL) was added to hydrazine monohydrate (1.29 mL, 26.6 mmol, 16.0 eq) and the mixture stirred at RT overnight. The solvents were evaporated under reduced pressure. Purification via reversed phase column chromatography yielded mixed fractions. Combined fractions containing product were purified via flash chromatography.

Purification via flash chromatography (MeOH in DCM, following a gradient from 0% to 15%) yielded 51 (97.0 mg, 18%).

### Example 52—Synthesis of diethyl ((6-(5-aminopyrimidin-2-yl)-1,2,4,5-tetrazin-3-yl)methyl)phosphonate (52)

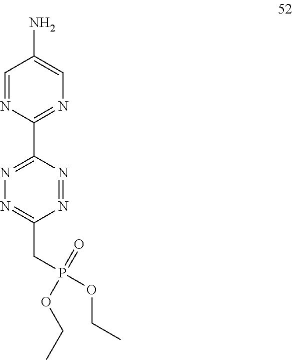 [SMILES: CCOP(=O)(Cc1nnc(-c2ncc(N)cn2)nn1)OCC]

To a solution of 51 (97.0 mg, 296 μmol, 1.00 eq) in THF/MeOH (9:1, 3 mL) was added p-benzoquinone (39.4 mg, 365 μmol, 1.23 eq) and the mixture stirred at RT for 5 min. It was diluted with DCM and the reaction quenched by addition of H₂O and a saturated aqueous solution of NaHCO₃. It was extracted with EtOAc (3 x), the combined organic phases dried over sodium sulfate and the solvents evaporated under reduced pressure.

Purification via flash chromatography (MeOH in DCM, following a gradient from 0% to 30%) yielded 52 (33.0 mg, 34%).

### Example 53—Synthesis of ((6-(5-aminopyrimidin-2-yl)-1,2,4,5-tetrazin-3-yl)methyl)phosphonic Acid (53)

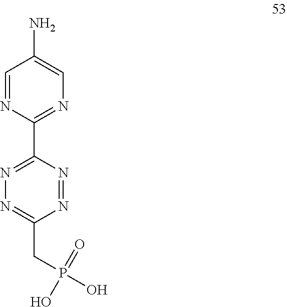 [SMILES: Nc1cnc(-c2nnc(CP(=O)(O)O)nn2)nc1]

To a solution of 52 (100 mg, 307 μmol, 1.00 eq) in DMF (1 mL) was added trimethylsilyl bromide (203 μL, 1.54 mmol, 5.00 eq) at 0° C. and the mixture stirred at RT overnight. Water was added and the solvents were evaporated under reduced pressure. Purification via reversed phase column chromatography yielded 53 (36.0 mg, 44%).

### Example 54—Synthesis of ((6-(5-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2-oxa-15,16-dithia-5,8,11-triazanonadecane-19-amido)pyrimidin-2-y)-1,2,4,5-tetrazin-3-yl)methyl)phosphonic Acid (54)

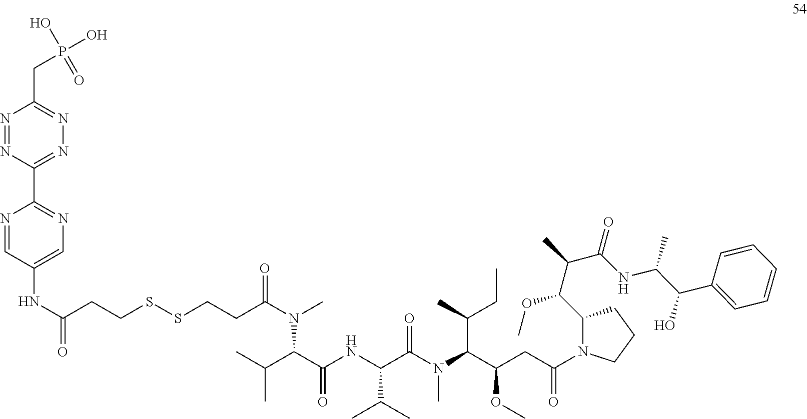 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCSSCCC(=O)Nc1cnc(-c2nnc(CP(=O)(O)O)nn2)nc1)C(C)C]

To a solution of 47 (10.0 mg, 13.2 μmol, 1.00 eq) and HATU (4.17 mg, 11.0 μmol, 1.00 eq) in DMF (0.5 mL) was added triethylamine (1.5 μL, 11.0. μmol, 1.00 eq) and the mixture stirred for 1 h at RT. Afterwards 53 (4.60 mg, 13.2 μmol, 1.20 eq) and triethylamine (1.53 μL, 11.0 μmol, 1.00 eq) was added and the mixture stirred at RT overnight. The reaction mixture was directly subjected to HPLC purification.

Purification via HPLC yielded 54 (5.0 mg, 39%).

### Example 55—Synthesis of 2-(2-((6-(6-((diethoxyphosphoryl)methyl)-1,2,4,5-tetrazin-3-yl)pyridin-3-yl)amino)-2-oxoethoxy)acetic Acid (55)

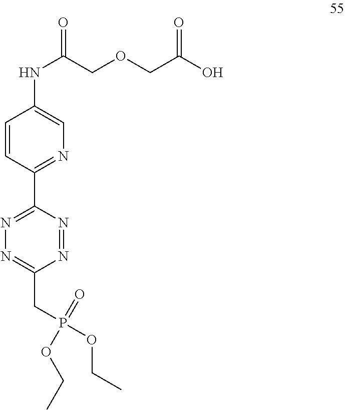 [SMILES: CCOP(=O)(Cc1nnc(-c2ccc(NC(=O)COCC(=O)O)cn2)nn1)OCC]

To a solution of 18 (64.8 mg, 200 μmol, 1.00 eq) and diglycolic anhydride (27.8 mg, 240 μmol, 1.20 eq) in DMF (2 mL) was added 4-(dimethylamino)pyridine (2.44 mg, 20.0 μmol, 0.100 eq) and the mixture stirred at RT overnight. The solvents were evaporated under reduced pressure.

Purification via reverse phase column chromatography yielded 55 (72.0 mg, 82%) as pink oil.

### Example 56—Synthesis of 4-((6-(6-((diethoxyphosphoryl)methyl)-1,2,4,5-tetrazin-3-yl)pyridin-3-yl)amino)-4-oxobutanoic Acid (56)

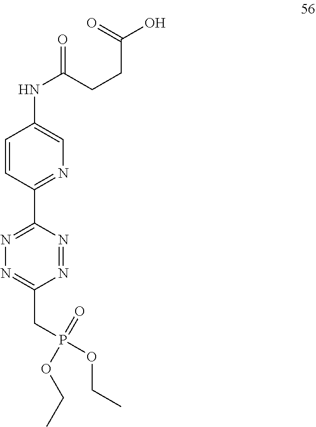 [SMILES: CCOP(=O)(Cc1nnc(-c2ccc(NC(=O)CCC(=O)O)cn2)nn1)OCC]

A solution of 18 (60.0 mg, 185 μmol, 1.00 eq) and succinic anhydride (22.2 mg, 222 μmol, 1.20 eq) in CHCl₃ (0.4 mL) was stirred at 60° C. overnight. The reaction mixture was diluted with DCM. The pink precipitate was filtered off and washed with DCM. 56 (69.0 mg, 88%) was isolated as pink solid.

### Example 57—Synthesis of tert-butyl (2-((6-(6-((diethoxyphosphoryl)methyl)-1,2,4,5-tetrazin-3-yl)pyridin-3-yl)amino)-2-oxoethyl)carbamate (57)

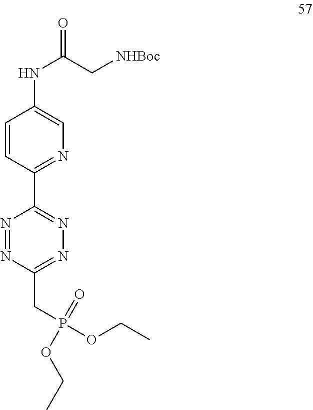 [SMILES: CCOP(=O)(Cc1nnc(-c2ccc(NC(=O)CNC(=O)OC(C)(C)C)cn2)nn1)OCC]

To a solution of N-Boc-glycine (486 mg, 2.78 mmol, 2.00 eq) in THF (5 mL) was added N-Methylmorpholine (763 μL, 6.94 mmol, 5.00 eq) and isobutyl chloroformate (360 μL, 2.78 mmol, 2.00 eq) at 0° C. and the mixture was stirred for 5 min. Then 18 (450 mg, 1.39 mmol, 1.00 eq) was added at 0° C. and the mixture was stirred at RT overnight Water and EtOAc were added, the phases were separated and the aqueous phase was extracted with EtOAc (2×). The combined organic phases were washed with a saturated aqueous solution of NaHCO₃, dried over Na₂SO₄ and the solvents evaporated under reduced pressure.

Purification via reversed phase chromatography yielded 57 (300 mg, 45%).

### Example 58—Synthesis of ((6-(5-(2-aminoacetamido)pyridin-2-yl)-1,2,4,5-tetrazin-3-yl)methyl)phosphonic Acid (58)

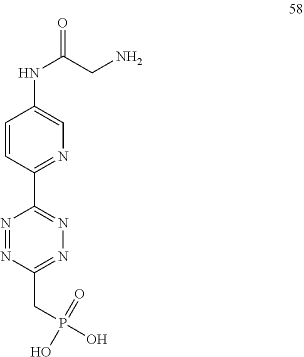 [SMILES: NCC(=O)Nc1ccc(-c2nnc(CP(=O)(O)O)nn2)nc1]

To a solution of 57 (200 mg, 415 μmol, 1.00 eq) in DMF (1 mL) was added trimethylsilyl bromide (274 μL, 2.08 mmol, 5.00 eq) at 0° C. and the mixture stirred at RT overnight. MeOH and water were added and the solvents were evaporated under reduced pressure.

Purification via reversed phase column chromatography yielded 58 (66.0 mg, 49%).

### Example 59—Synthesis of ((6-(5-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12,19-tetraoxo-2-oxa-15,16-dithia-5,8,11,20-tetraazadocosan-22-amido)pyridin-2-yl)-1,2,4,5-tetrazin-3-yl)methyl)phosphonic Acid (59)

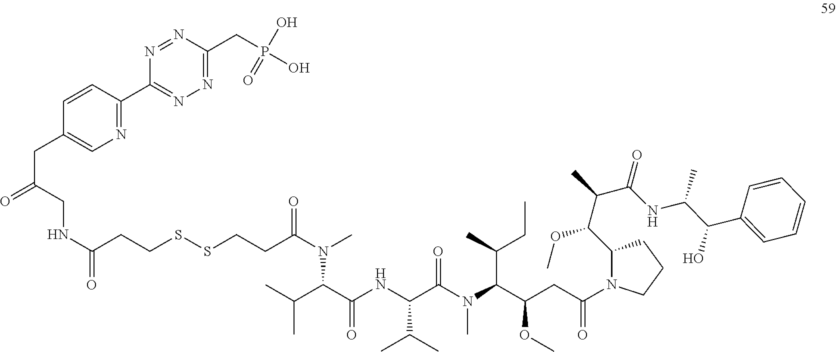 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCSSCCC(=O)NCC(=O)Cc1ccc(-c2nnc(CP(=O)(O)O)nn2)nc1)C(C)C]

To a solution of 47 (10.0 mg, 11.0 μmol, 1.00 eq) and HATU (4.17 mg, 11.0 μmol, 1.00 eq) in DMF (0.5 mL) was added triethylamine (1.53 μL, 11.0 μmol, 1.00 eq) and the mixture stirred for 1 h at RT. Afterwards 58 (5.35 mg, 13.2 μmol, 1.20 eq) and triethylamine (1.53 μL, 11.0 μmol, 1.00 eq) was added, the mixture was stirred overnight at RT and directly subjected to HPLC purification.

Purification via HPLC yielded 59 (1.7 mg, 13%).

### Example 60—Synthesis of tert-butyl (2-((2-(6-((diethoxyphosphoryl)methyl)-1,2,4,5-tetrazin-3-yl)pyrimidin-5-yl)amino)-2-oxoethyl)carbamate (60)

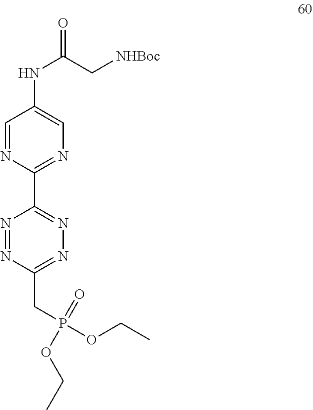 [SMILES: CCOP(=O)(Cc1nnc(-c2ncc(NC(=O)CNC(=O)OC(C)(C)C)cn2)nn1)OCC]

To a solution of N-Boc-glycine (255 mg, 1.46 mmol, 2.00 eq) in THF (5 mL) was added N-methylmorpholine (0.401 mL, 3.64 mmol, 5.00 eq) and isobutyl chloroformate (0.189 mL, 1.46 mmol, 2.00 eq) at 0° C. and the mixture was stirred for 5 min. Then 52 (237 mg, 0.729 mmol, 1.00 eq) was added and the mixture was stirred at RT overnight. Water and EtOAc were added, the phases were separated and the aqueous phase was extracted with EtOAc (2×). The combined organic phases were washed with a saturated aqueous solution of NaHCO₃, dried over Na₂SO₄ and the solvents evaporated under reduced pressure.

Purification via reversed phase column chromatography yielded 60 (152 mg, 43%).

### Example 61—Synthesis of diethyl ((6-(5-(2-aminoacetamido)pyrimidin-2-y)-1,2,4,5-tetrazin-3-yl)methyl)phosphonate (61)

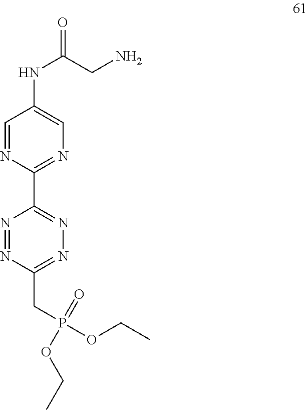 [SMILES: CCOP(=O)(Cc1nnc(-c2ncc(NC(=O)CN)cn2)nn1)OCC]

To a solution of 60 (130 mg, 0.269 mmol, 1.00 eq) in DCM (2 mL) was added TFA (208 μl, 2.70 mmol, 10.0 eq) at RT and the mixture stirred overnight. The solvents were removed under reduced pressure.

Evaporation yielded 61 (79 mg, 59%).

### Example 62—Synthesis of ((6-(5-(2-aminoacetamido)pyrimidin-2-yl)-1,2,4,5-tetrazin-3-yl)methyl)phosphonic Acid (62)

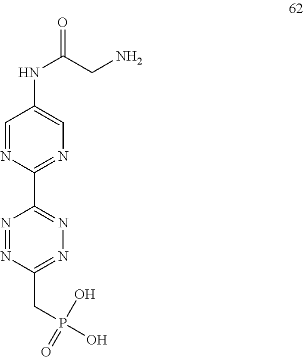 [SMILES: NCC(=O)Nc1cnc(-c2nnc(CP(=O)(O)O)nn2)nc1]

To a solution of 61 (67.0 mg, 135 μmol, 1.00 eq) in DMF (0.5 mL) was added TMS-Br (178 μL, 1.35 mmol, 10.0 eq) at 0° C. and the mixture stirred at RT overnight MeOH and water were added and the solvents were evaporated under reduced pressure.

Purification via reversed phase column chromatography yielded 62 (92 mg) as a mixture with DMF. The product was used like this for subsequent reactions.

### Example 63—Synthesis of ((6-(5-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidine-1-yl)-2-oxoethyl)-7,10-diisopropyl-5,11-dimethyl-6,9,12,19-tetraoxo-2-oxa-15,16-dithia-5,8,11,20-tetraazadocosan-22-amido)pyrimidin-2-yl)-1,2,4,5-tetrazin-3-yl)methyl)phosphonic Acid (63)

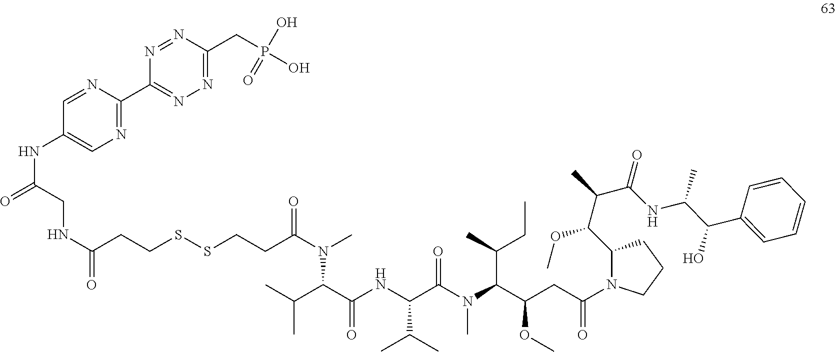 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCSSCCC(=O)NCC(=O)Nc1cnc(-c2nnc(CP(=O)(O)O)nn2)nc1)C(C)C]

To a solution 47 (10.0 mg, 11.0 μmol, 1.00 eq) and HATU (4.17 mg, 11.0 μmol, 1.00 eq) in DMF (0.5 mL) was added triethylamine (1.53 μL, 11.0 μmol, 1.00 eq) and the mixture stirred for 1 h at RT. Afterwards 62 (5.37 mg, 13.18 μmol, 1.20 eq) Triethylamine (1.53 μL, 11.0 μmol, 1.00 eq) was added, the mixture stirred at RT overnight and directly subjected to HPLC purification.

Purification via HPLC yielded 63 (1.2 mg, 9%).

### Example 64—Synthesis of (6-(6-(5-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2-oxa-15,16-dithia-5,8,11-triazanonadecane-19-amido)pyridin-2-y)-1,2,4,5-tetrazin-3-yl)pyridin-3-yl)phosphonic Acid (64)

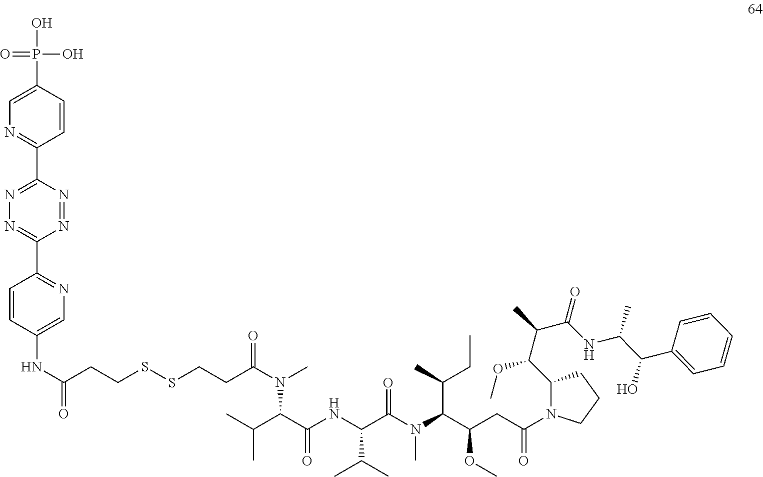 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCSSCCC(=O)Nc1ccc(-c2nnc(-c3ccc(P(=O)(O)O)cn3)nn2)nc1)C(C)C]

To a solution 47 (2.75 mg, 3.02 μmol, 1.00 eq) and HATU (1.15 mg, 3.02 μmol, 1.00 eq) in DMF (0.5 mL) was added triethylamine (0.42 μL, 3.02 μmol, 1.00 eq) and the mixture stirred for 1 h at RT. Afterwards 37 (1.00 mg, 3.02 μmol, 1.00 eq) and triethylamine (0.42 μL, 3.02 μmol, 1.00 eq) were added, the mixture stirred at RT overnight and directly subjected to HPLC purification.

Purification via HPLC yielded 64 (1 mg, 27%).

### Example 65—Synthesis of (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(3-(3-(6-(4-phosphonophenyl)-1,2,4,5-tetrazin-3-yl)propanamido)propanamido)phenoxy)-3,4,5-trihydroxytetrahydro-2H-pyran-2-carboxylic Acid (65)

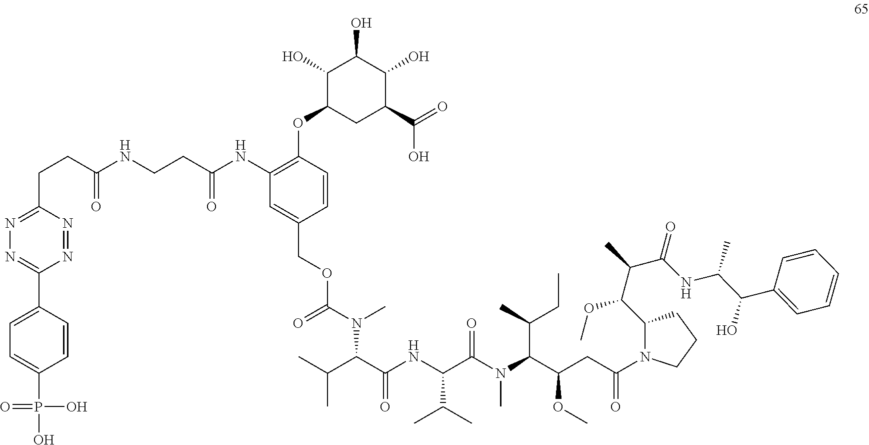 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2C[C@H](C(=O)O)[C@@H](O)[C@H](O)[C@H]2O)c(NC(=O)CCNC(=O)CCc2nnc(-c3ccc(P(=O)(O)O)cc3)nn2)c1)C(C)C]

To a solution of 32 (2.00 mg, 4.91 μmol, 1.00 eq) and 44 (4.44 mg, 3.93 μmol, 0.800 eq) in DMF (0.3 mL) was added triethylamine (0.753 μL, 5.40 μmol, 1.10 eq), the mixture stirred for 3 h at RT and directly subjected to HPLC purification.

Purification via HPLC yielded 65 (4.4 mg, 63%).

### Example 66—Synthesis of (2S,3S,4S,5R,6S)-6-(4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)-2-(3-(4-(6-(phosphonomethyl)-1,2,4,5-tetrazin-3-yl)benzamido)propanamido)phenoxy)-3,4,5-trihydroxytetrahydro-2H-pyran-2-carboxylic Acid (66)

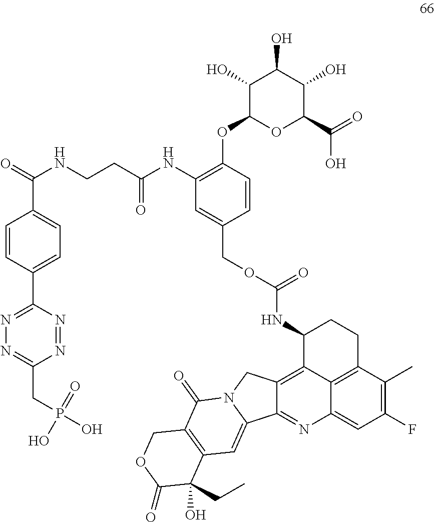 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)c2ccc(-c4nnc(CP(=O)(O)O)nn4)cc2)c1)CC3]

A solution of 12 (0.928 mg, 2.36 μmol, 1.00 eq) and 45 (2.00 mg, 2.36 μmol, 1.00 eq) was added triethylamine (0.362 μL, 2.60 μmol, 1.10 eq), stirred for 3 h at RT and directly subjected to HPLC purification.

Purification via HPLC yielded 66 (0.6 mg, 23%).

### Example 67—Synthesis of ((6-(4-(((S)-1-(((S)-1-(((S)-4-amino-1-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1,4-dioxobutan-2-yl)amino)-1-oxopropan-2-yl)amino)-1-oxopropan-2-yl)carbamoyl)phenyl)-1,2,4,5-tetrazin-3-yl)methyl)phosphonic Acid (67)

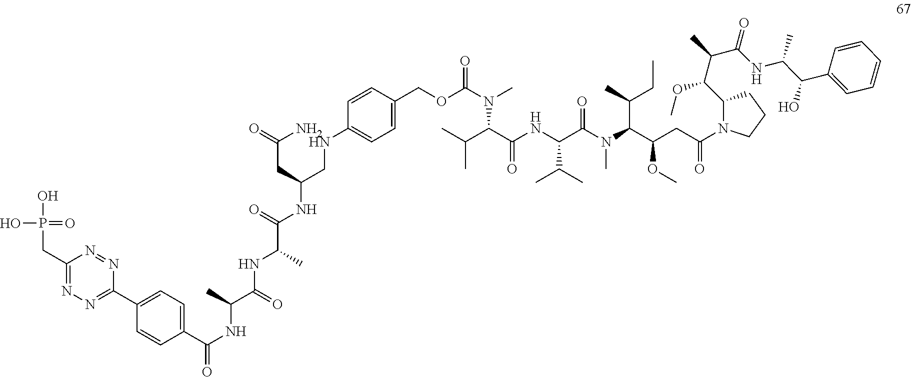 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC[C@H](CC(N)=O)NC(=O)[C@H](C)NC(=O)[C@H](C)NC(=O)c2ccc(-c3nnc(CP(=O)(O)O)nn3)cc2)cc1)C(C)C]

To a solution of 12 (1.40 mg, 3.56 μmol, 1.00 eq) and 46 (4.00 mg, 3.56 μmol, 1.00 eq) in DMF (0.5 mL) was added triethylamine (0.545 μL, 3.91 μmol, 1.10 eq), stirred for 3 h at RT and directly subjected to HPLC purification.

Purification via HPLC yielded 67 (1.0 mg, 20%).

### Example 68—Synthesis of 3-(6-((diethoxyphosphoryl)methyl)-1,2,4,5-tetrazin-3-yl)propanoic Acid (68)

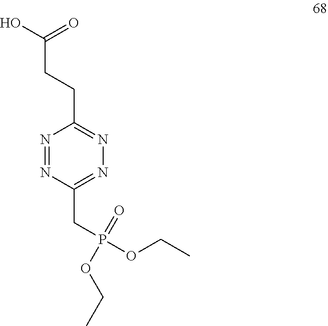 [SMILES: CCOP(=O)(Cc1nnc(CCC(=O)O)nn1)OCC]

A solution of 3-cyanopropionic acid (400 mg, 4.04 mmol, 1.00 eq), diethyl cyanomethylphosphonate (2.61 mL, 16.1 mmol, 4.00 eq) and 3-mercaptopropionic acid (1.06 mL, 12.1 mmol, 3.00 eq) in EtOH (1 mL) was added to hydrazine monohydrate (3.13 mL, 64.6 mmol, 16.0 eq) at 50° C. and the mixture was stirred at this temperature for 5 h. Afterwards the reaction mixture was cooled to 0° C., NaNO₂ (6.86 g, 80.7 mmol, 20.0 eq) in H₂O was added and it was acidified to pH-3 via dropwise addition of 1 M HCl_{aq}, The formed precipitate was filtered off and the aqueous phase was extracted with EtOAc (2×). The combined organic phases were dried over Na₂SO₄ and the solvents evaporated under reduced pressure.

Purification via reversed phase column chromatography yielded mixed fractions. Fractions containing product were combined, the solvents evaporated under reduced pressure and the residue dissolved in water (5 mL). The formed precipitate was filtered off and the filtrate lyophilized.

68 (140 mg, 11%) was isolated as pink oil.

### Example 69—Synthesis of 2,5-dioxopyrrolidin-1-yl 3-(6-((diethoxyphosphoryl)methyl)-1,2,4,5-tetrazin-3-yl)propanoate (69)

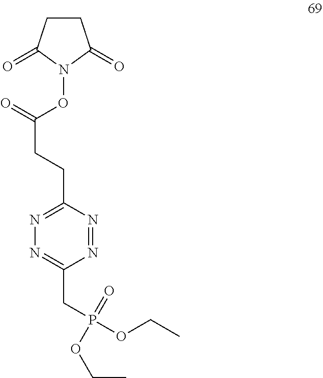 [SMILES: CCOP(=O)(Cc1nnc(CCC(=O)ON2C(=O)CCC2=O)nn1)OCC]

To a solution of 68 (120 mg, 0.394 mmol, 1.00 eq) and TSTU (142 mg, 0.473 mmol, 1.20 eq) in DCM (0.5 mL) was added DIPEA (82.4 μL, 0.473 mmol, 1.20 eq) and the mixture was stirred at RT overnight. The solvents were evaporated under reduced pressure.

Purification via reversed phase column chromatography yielded 69 (88.0 mg, 56%).

### Example 70—Synthesis of 4(S)-4-amino-2-((S)-2-((S)-2-(3-(6-((diethoxyphosphoryl)methyl)-1,2,4,5-tetrazin-3-yl)propanamido)propanamido)propanamido)-4-oxobutanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate (70)

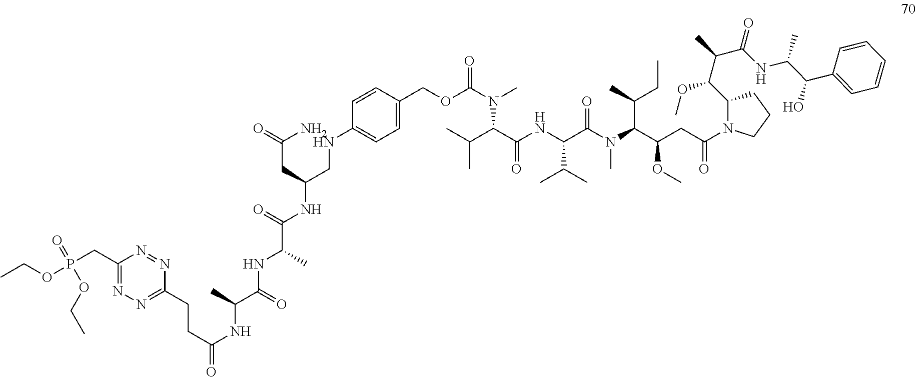 [SMILES: CCOP(=O)(Cc1nnc(CCC(=O)N[C@@H](C)C(=O)N[C@@H](C)C(=O)N[C@H](CNc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)CC(N)=O)nn1)OCC]

To a solution of 69 (1.43 mg, 3.56 μmol, 1.00 eq) and 46 (4.00 mg, 3.56 μmol, 1.00 eq) in DMF (0.3 mL) was added triethylamine (0.992 μL, 7.12 μmol, 2.00 eq), the mixture stirred for 3 h at room temperature and directly subjected to HPLC purification.

Purification via HPLC yielded 70 (1.8 mg, 36%).

### Example 71—Synthesis of (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(3-(3-(6-((diethoxyphosphoryl)methyl)-1,2,4,5-tetrazin-3-yl)propanamido)propanamido)phenoxy)-3,4,5-trihydroxytetrahydro-2H-pyran-2-carboxylic Acid (71)

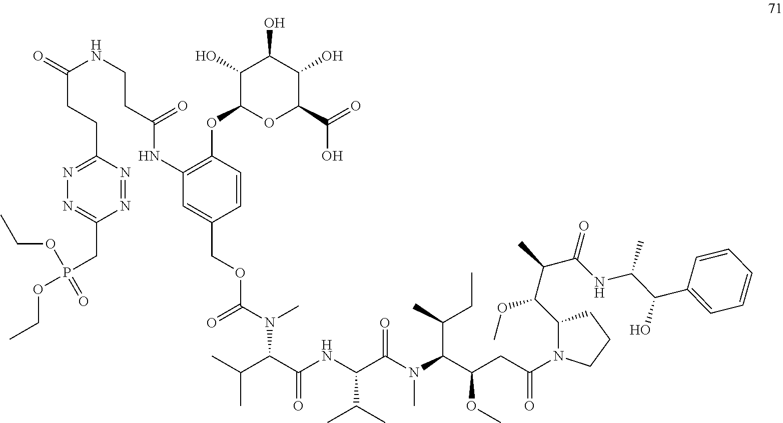 [SMILES: CCOP(=O)(Cc1nnc(CCC(=O)NCCC(=O)Nc2cc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)ccc2O[C@@H]2O[C@H](C(=O)O)[C@@H](O)[C@H](O)[C@H]2O)nn1)OCC]

To a solution of 69 (1.56 mg, 3.89 μmol, 1.10 eq) and 45 (4.00 mg, 3.54 μmol, 1.00 eq) in DMF (0.3 mL) was added triethylamine (0.986 μL, 7.08 μmol, 2.00 eq), the mixture stirred for 3 h at room temperature and directly subjected to HPLC purification.

Purification via HPLC yielded 71 (1.2 mg, 24%).

### Example 72—Synthesis of ((6-(3,5-bis(((2,5-dioxopyrrolidin-1-yl)oxy)carbonyl)phenyl)-1,2,4,5-tetrazin-3-yl)methyl)phosphonic Acid (72)

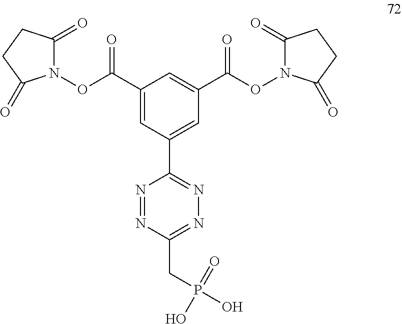 [SMILES: O=C(ON1C(=O)CCC1=O)c1cc(C(=O)ON2C(=O)CCC2=O)cc(-c2nnc(CP(=O)(O)O)nn2)c1]

To a solution of 15 (135 mg, 229 μmol, 1.00 eq) in DMF (2.2 mL) was added TMS-Br (151 μL, 1.14 mmol, 5.00 eq) at 0° C. and the mixture stirred at RT overnight. Water and MeCN were added and the mixture was directly subjected to purification via reverse phase column chromatography.

Purification via reverse phase column chromatography yielded 72 (11.2 mg, 9%) as pink solid.

### Example 73—Synthesis of diethyl ((6-(3,5-bis(((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,15,18,21,24,27,30,33,36-nonaoxa-5,8,11-triazaoctatriacontan-38-yl)carbamoyl)phenyl)-1,2,4,5-tetrazin-3-yl)methyl)phosphonate (73)

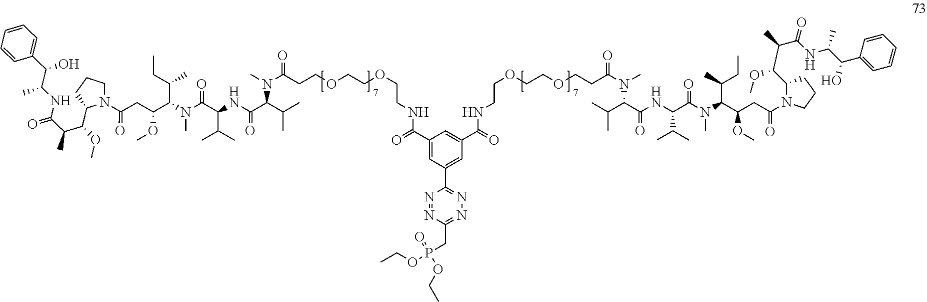

To a solution of 16 (23.3 mg, 18.7 μmol, 1.00 eq) and MMAE (32.3 mg, 45.0 μmol, 2.40 eq) in DMF (0.2 mL) was added HATU (21.4 mg, 56.2 μmol, 3.00 eq) and triethylamine (13.0 μL, 93.7 μmol, 5.00 eq) and the mixture stirred for 2 h at RT. Afterwards water was added and the mixture directly subjected to HPLC purification.

Purification via HPLC yielded 73 (22.5 mg, 45%) as pink oil.

### Example 74—Synthesis of ((6-(3,5-bis(((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,15,18,21,24,27,30,33,36-nonaoxa-5,8,11-triazaoctatriacontan-38-yl)carbamoyl)phenyl)-1,2,4,5-tetrazin-3-yl)methyl)phosphonic Acid (74)

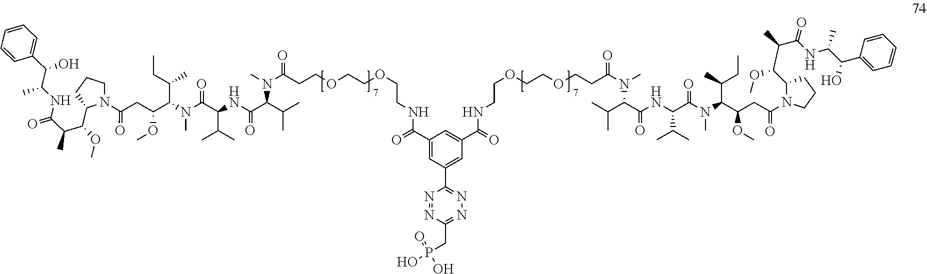

To a solution of 73 (22.0 mg, 8.32 μmol, 1.00 eq) in DMF (0.2 mL) was added TMS-Br (11.0 μL, 83.2 μmol, 10.0 eq) at 0° C. and the mixture stirred at RT for 6 h. Afterwards 20 μL of TMS-Br were added and the mixture stirred overnight. Afterwards 40 μL of TMS-Br were added and the mixture stirred for 8 d. Water was added and the mixture was directly subjected to purification via HPLC.

Purification via HPLC yielded 74 (3.5 mg, 16%) as pink powder.

### Example 75—Synthesis of diethyl ((6-(3,5-bis((2-aminoethyl)carbamoyl)phenyl)-1,2,4,5-tetrazin-3-yl)methyl)phosphonate (75)

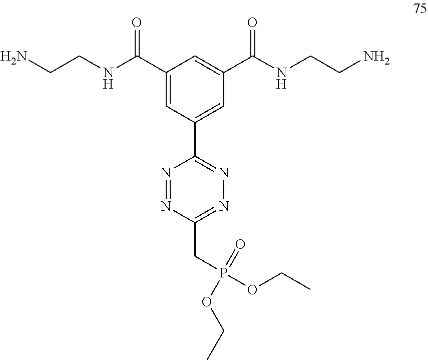 [SMILES: CCOP(=O)(Cc1nnc(-c2cc(C(=O)NCCN)cc(C(=O)NCCN)c2)nn1)OCC]

To a solution of 1,2-diaminoethane (44.8 μL, 671 μmol, 6.00 eq) in DMF (0.6 mL) was added a solution of 15 (66.0 mg, 112 μmol, 1.00 eq) in DMF (0.6 mL) and the mixture stirred at RT for 15 min. Afterwards 0.5 mL 1 M HCl_{aq} was added and the mixture directly subjected to purification via HPLC.

Purification via HPLC yielded 75 (8.0 mg, 15%) as pink oil.

### Example 76—Synthesis of ((6-(3,5-bis((2-aminoethyl)carbamoyl)phenyl)-1,2,4,5-tetrazin-3-yl)methyl)phosphonic Acid (76)

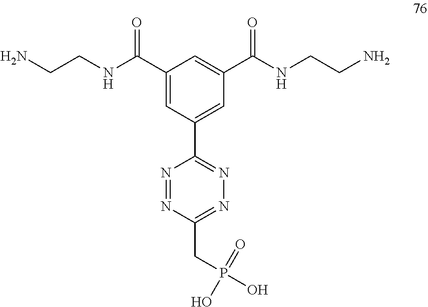 [SMILES: NCCNC(=O)c1cc(C(=O)NCCN)cc(-c2nnc(CP(=O)(O)O)nn2)c1]

To a solution of 75 (8.00 mg, 16.7 μmol, 1.00 eq) in DMF (0.2 mL) was added TMS-Br (44.0 μL, 333 μmol, 20.0 eq) at 0° C. and the mixture stirred at RT for 2 h. Water was added and the mixture was directly subjected to purification via HPLC.

Purification via HPLC yielded 76 (4.0 mg, 57%) as pink solid.

### Example 77—Synthesis of 3-((3-(((S)-1-(((14S,16S,32R,33S,2S,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl)oxy)-1-oxopropan-2-yl)methyl)amino)-3-oxopropyl)disulfanyl)propanoic Acid (77)

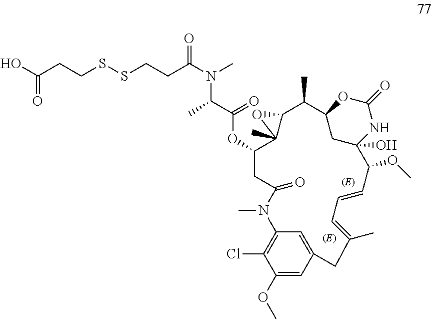 [SMILES: COc1cc2cc(c1Cl)N(C)C(=O)C[C@H](OC(=O)[C@H](C)N(C)C(=O)CCSSCCC(=O)O)[C@]1(C)O[C@@H]1[C@@H](C)[C@@H]1C[C@@](O)(NC(=O)O1)[C@H](OC)/C=C/C=C(\C)C2]

To a solution of 3,3′-dithiodipropionic Acid (19.4 mg, 92.3 μmol, 2.00 eq) and HATU (17.5 mg, 46.1 μmol, 1.00 eq) in DMF (0.5 mL) was added DIPEA (8.04 μL, 46.1 μmol, 1.00 eq) and the mixture stirred for 1 h at RT. Afterwards Maytansinol-Ala (30.0 mg, 46.1 μmol, 1.00 eq) and DIPEA (8.04 μL, 46.1 μmol, 1.00 eq) was added, the reaction mixture stirred at RT overnight and directly subjected to HPLC purification.

Purification via HPLC yielded 77 (15.2 mg, 68%)

### Example 78—Synthesis of ((6-(5-(3-((3-(((S)-1-(((14S,16S,32R,33S,2S,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl)oxy)-1-oxopropan-2-yl)methyl)amino)-3-oxopropyl)disulfanyl)propanamido)pyrimidin-2-yl)-1,2,4,5-tetrazin-3-yl)methyl)phosphonic Acid (78)

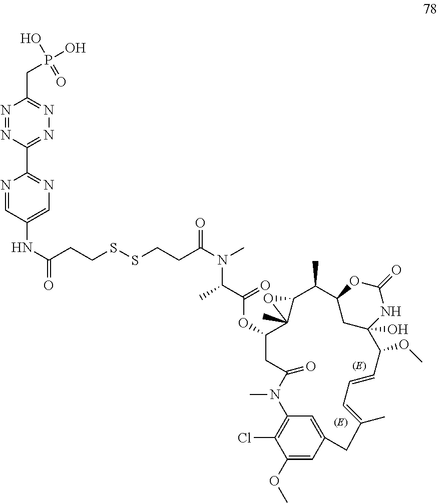 [SMILES: COc1cc2cc(c1Cl)N(C)C(=O)C[C@H](OC(=O)[C@H](C)N(C)C(=O)CCSSCCC(=O)Nc1cnc(-c3nnc(CP(=O)(O)O)nn3)nc1)[C@]1(C)O[C@@H]1[C@@H](C)[C@@H]1C[C@@](O)(NC(=O)O1)[C@H](OC)/C=C/C=C(\C)C2]

To a solution of 77 (8.00 mg, 9.50 μmol, 1.00 eq) and HATU (3.61 mg, 9.50 μmol, 1.00 eq) in DMF (0.50 mL) was added triethylamine (1.32 μL, 9.50 μmol, 1.00 eq) and the mixture stirred for 1 h at RT. Afterwards 53 (3.82 mg, 14.25 μmol, 1.50 eq) and triethylamine (1.32 μL, 9.50 μmol, 1.00 eq) was added and the mixture stirred at RT for 3 h. The reaction mixture was directly subjected to HPLC purification.

Purification via HPLC yielded 78 (mixture with free 77) (3.20 mg (75% purity), 22%).

### Example 79—Synthesis of ((6-(2(3-((3-(((S)-1-(((14S,16S,32R,33S,2S,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl)oxy)-1-oxopropan-2-yl)methyl)amino)-3-oxopropyl)disulfanyl)propanamido)ethyl)-1,2,4,5-tetrazin-3-yl)methyl)phosphonic Acid (79)

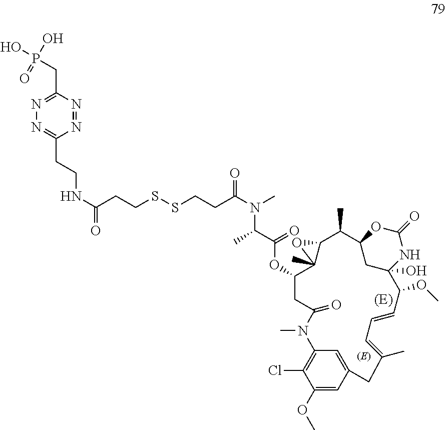 [SMILES: COc1cc2cc(c1Cl)N(C)C(=O)C[C@H](OC(=O)[C@H](C)N(C)C(=O)CCSSCCC(=O)NCCc1nnc(CP(=O)(O)O)nn1)[C@]1(C)O[C@@H]1[C@@H](C)[C@@H]1C[C@@](O)(NC(=O)O1)[C@H](OC)/C=C/C=C(\C)C2]

To a solution of 77 (7.20 mg, 8.55 μmol, 1.00 eq) and HATU (3.25 mg, 8.55 μmol, 1.00 eq) in DMF (0.50 mL) was added triethylamine (1.19 μL, 8.55 μmol, 1.00 eq) and the mixture stirred for 1 h at RT. Afterwards 50 (3.82 mg, 14.25 μmol, 1.50 eq) and triethylamine (1.19 μL, 8.55 μmol, 1.00 eq) was added and the mixture stirred at RT for 3 h. The reaction mixture was directly subjected to HPLC purification.

Purification via HPLC yielded 79 (0.90 mg, 10%).

### C) Preparation of ADC and Cytotoxicity Assay

### Preparation Example 1—Preparation of Trastuzumab A132TCO*A

Trastuzumab A132TCO*A was expressed in insect cells (Spodoptera frugiperda cells, Sf21) utilizing the baculovirus based transduction system. Therefore, the gene of the heavy chain of Trastuzumab containing an amber stop codon at position A132 and a C-terminal 6-His tag, was cloned into pACEBac-DUAL plasmid (as for example described in WO 2017/093254) into the first multicloning site. The gene of the light chain of Trastuzumab was cloned without further modifications into the second multicloning site of the plasmid.

The resulting plasmid, pACEBacDUAL-Trastuzumab heavy A132TAG-6His-light was transformed into DH10MultiBac-TAG cells harboring a Bacmid with the expression cassette for NES-PyIRS^{AF} (as for example described in WO2018/069481) as well U6(Sf21)-tRNA^{PyI} (as for example described in WO 2017/093254) in the backbone of the Bacmid. The transformation results in the integration of the plasmid into the Bacmid-DNA. After preparation of Bacmid-DNA, insect cells (Sf21 cells) were transfected with the Bacmid-DNA. After three days, the V₀-Virus was harvested and used to transduce a fresh batch of Sf21 cells, resulting in the production of V₁-Virus. This Virus was used to transduce a large expression culture (liter scale). After adding TCO*A-Lys the expression was carried out for 4 days. The cells were harvested at 500 rcf using a Beckman rotor (JLA 8.1000) for 1 hour at 4° C.

The cells were resuspended in lysis buffer (4×PBS, 0.2 mM TCEP, 1 mM PMSF, 5 mM Imidazole, pH 8) and sonicated three times for 30 seconds on ice. After a centrifugation step at 27143.1 RCF for 1 hour at 4° C. in a fixed angle rotor (JA 25.50, Beckman), the cleared lysate was incubated on nickel beads for 1 hour at 4° C. on a rocker. The nickel beads were collected in a polypropylene (PP)-column (Qiagen, Cat. No.: 34964) and washed with lysis buffer, containing 10 nM Imidazole. Trastuzumab was eluted from the nickel beads using 500 mM Imidazole in the lysis buffer. The elution fraction was loaded on a MabSelect PrismA column equilibrated in Buffer (0.02 M Na₂PO₄, 0.15 M NaCl, pH 7.2). After a washing step with Buffer A, Trastuzumab was eluted from the column using a gradient up to 100% Buffer B (0.1 M sodium citrate, pH 3.2). Fractions were collected, which contained 1 M Tris pH 10 to neutralize the eluting sample. After analyzing the fractions on SDS-PAGE, the fractions containing Trastuzumab were pooled and concentrated using an amicon filter device (30 kDa cutoff). The sample was further purified using a Superdex S200 (10/30) column equilibrated in 1×PBS buffer. Fractions were collected and analyzed on SDS-PAGE. After concentrating the corresponding fractions, the sample was used for labeling with the cytotoxic payload.

### Preparation Example 2—Preparation of Trastuzumab-TCO*A-5

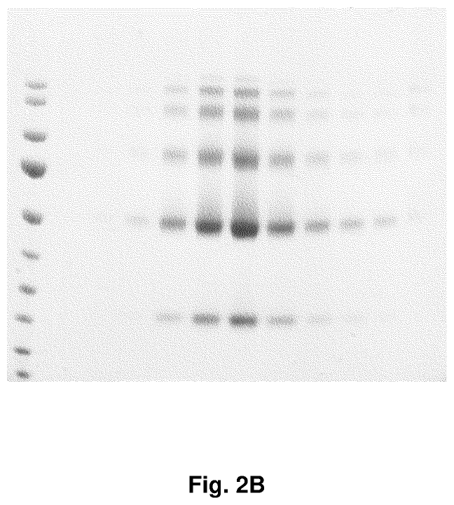

10 nmol Trastuzumab A132TCO*A were incubated in 1×PBS with 40 nmol of phosphonate-tetrazine-MMAE (5) at 37° C. shaking at 600 rpm for 1 hour. After washing the reaction mix in a filter device (Amicon Filter device, 30 kDa cutoff) with 1×PBS to remove any unreacted drug. The ADC was further purified by size exclusion chromatography using a Superdex Increase S200 column, equilibrated in 1×PBS (FIG. 2 A). The collected fractions were analyzed by SDS-PAGE, stained with Coomassie blue (FIG. 2 B). The fractions containing the ADC were pooled and concentrated in an Amicon filter device.

### Preparation Example 3—Preparation of Trastuzumab-TCO*A-11

10 nmol Trastuzumab A132TCO*A were incubated in 1×PBS with 40 nmol phosphonate-tetrazine-Val-Ala-PAB-MMAE (11) at 37*C ° shaking at 600 rpm for 1 hour.

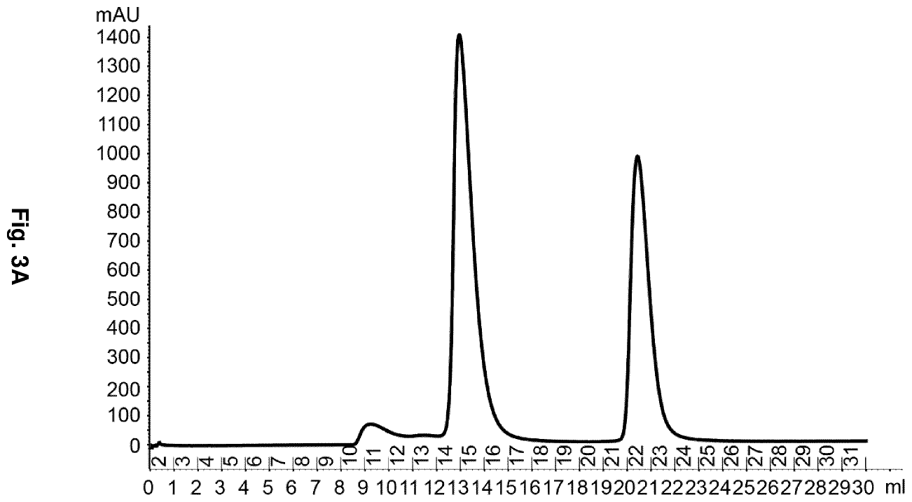

After washing the reaction mix in a filter device (Amicon Filter device, 30 kDa cutoff) with 1x PBS to remove any unreacted drug. The ADC was further purified by size exclusion chromatography using a Superdex Increase S200 column, equilibrated in 1×PBS (FIG. 3 A). The collected fractions were analyzed by SDS-PAGE, stained with Coomassie blue (FIG. 3 B). The fractions containing the ADC were pooled and concentrated in an Amicon filter device.

### D) Cytotoxicity Assays

### Assay Example 1—Cell Cytotoxicity Assay

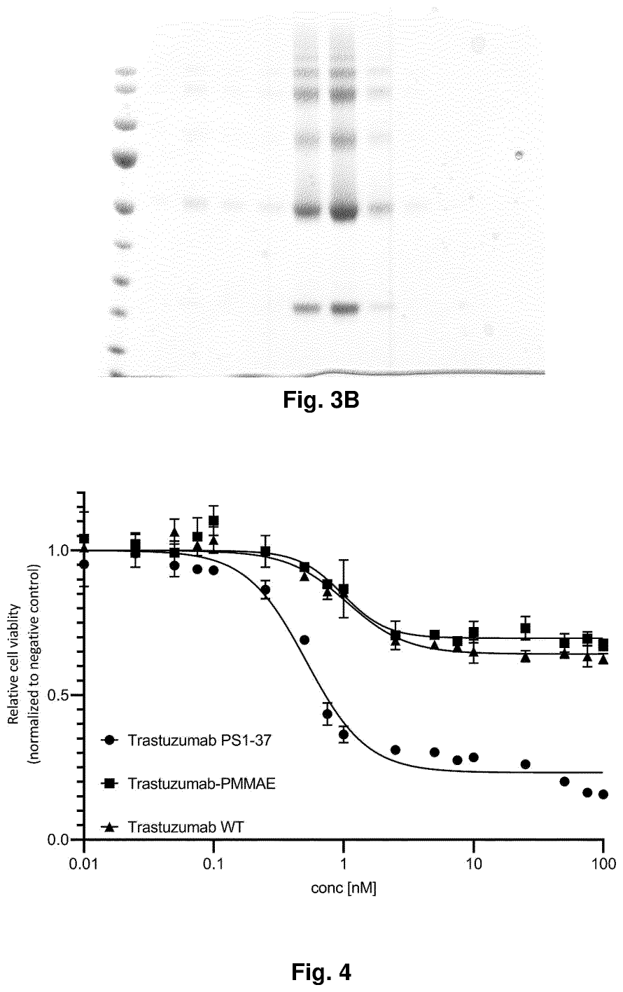

Cells of the breast cancer cell line SK-BR-3 were seeded at 5000 cell/well in a black 96-well plate two days prior ADC application. The concentration of the different ADCs or Abs (as negative control) were adjusted to 5 pM. Serial dilutions were prepared in the range from 0-100 nM. The medium from the 96-well plate was aspirated and replaced by the dilutions of the ADCs or Abs. As ADCs, Trastuzumab A132TCO*A-5 and Trastuzumab A132TCO*A-11 were tested as well as Trastuzumab WT, which did not have any modifications. After 5 days incubation, the plates were taken from the incubator, warmed up for 30 minutes at RT and 100 μl CellTiter-Glo® 2.0 Cell (Promega) was added to each well. The plates were shaken at 50 rpm on a rocker for 2 minutes and 10 minutes incubated at RT. Then the luminescence signal was read out using a plate reader. The luminescence signal was normalized to the measurement at time point 0 nM (negative control). The plot in FIG. 4 shows the normalized luminescence signal of the two ADC and Trastuzumab WT at different concentrations (in M).

The content of any document cross-referenced in this description is incorporated by reference.

1. A tetrazine compound of the general formula I 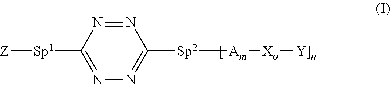
wherein
m is 0 or 1
n represents an integer selected from 1 and 2
is 0 or represents an integer selected from 1 or 2
A represents a cleavable linker moiety
Sp¹ and Sp² independently of each other represent a spacer moiety
X represents a self-immolative moiety
Y represents a payload residue (cargo) and
Z represents a phosphor and/or sulfur containing hydrophilic group;
in particular selected from (R¹O)₂P(O)—, (R^{1a}O)₂P(O)—O—, (R²O)₃P—O—, R³S(O)₂—, (R⁴O)S(O)₂O—, and (R^{4a}O)S(O)₂—,
wherein
residues R¹ to R⁴, R^{1a} and R^{4a} are same or different and independently of each other represent H or lower alkyl, in particular methyl or ethyl; and even more particularly H;
or a salt form of said phosphor and/or sulfur containing hydrophilic moieties.

2. The compound of claim 1, wherein Z is selected from one of the following hydrophilic groups:
(R¹O)₂P(O)—,(R^{1a}O)₂P(O)—O—, and (R²O)₃P—O—,
wherein
residues R¹, R² and R^{1a} are same or different and independently of each other represent H or lower alkyl, in particular methyl or ethyl;
and even more particularly H;
or a salt form of said phosphor containing hydrophilic moieties.

3. The compound of anyone of the preceding claims, wherein the spacer Sₚ¹ is absent or, more particularly, selected from
a) mono- or polycyclic optionally mono- or poly-substituted aromatic moieties having 6 to 14 ring carbon atoms, in particular 1,4-phenylene,
wherein
said one or more optional substituents are independently of each other selected from -Hal, —CHal₃, —OH, —SH, —NR′₂, NO₂, —CN, —C(═O)R″, —C(═O)OR′″, alkyl, alkenyl, alkynyl, and alkoxy;
wherein
R′, R″ and R′″ independently of each other are selected from H and C₁- to C₄-alkyl
(Moiety M1);
b) heterocyclic residues of the general formula X 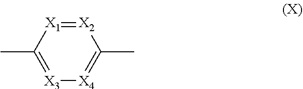
wherein
one, two or three, more particularly one or two of the ring moieties X₁ to X₄ represents N and the other represent >CH;
(Moiety M2);
c) linear or branched lower-alkylene, in particular —(CH₂)ₙ₁—,
wherein
n1 is an integer from 1 to 4; more particularly methylene;
(Moiety M3); and
d) combinations of at least two identical or, more particularly, different moieties, selected from M1, M2 and M3;
and/or
wherein the spacer Sₚ² is selected from
a) mono- or polycyclic optionally mono- or poly-substituted aromatic moieties having 6 to 14 ring carbon atoms, in particular 1,2-phenylene 1,3-phenylene or 1,4-phenylene; or a monocyclic moiety of formula 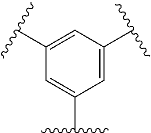 [SMILES: *c1cc(*)cc(*)c1]
more particularly 1,4-phenylene,
wherein
said one or more optional substituents are independently of each other selected from -Hal, —CHal₃, —OH, —SH, —NR′₂, NO₂, —CN, —C(═O)R″, —C(═O)OR′″, alkyl, alkenyl, alkynyl, and alkoxy;
wherein
R′, R″ and R′″ independently of each other are selected from H and C₁- to C₄-alkyl
(Moiety M1);
b) heterocyclic residues of the general formula X 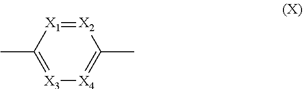
wherein
one, two or three, more particularly one or two of the ring moieties X₁ to X₄ represents N and the other represent >CH;
(Moiety M2);
c) linear or branched lower-alkylene, in particular —(CH₂)ₙ₁—, wherein n1 is an integer from 1 to 4; more particularly methylene;
(Moiety M3);
d) linear or branched polyalkylene oxide moieties, in particular selected from linear the moieties —((CH₂)ₓ₁-0)_{y1}— or —(O—(CH₂)ₓ₁)_{y1}— and the branched analogues thereof;
wherein
x1 independently of each other represent an integer selected from 1, 2, 3 or 4; in particular 1 or 2; and
y1 independently of each other represent an integer from 1 to 20, in particular 1 to 4;
(Moiety M4);
e) a heteroatom containing moiety selected from
—N(R″″)—,
—(CH₂)ₓ₂—N(R″″)—;
—N(R″″)—(CH₂)ₓ₃—C(O)O—;
—N(R″″)—(CH₂)ₓ₃—C(O)—;
—N(R″″)—C(O)O—(CH₂)ₓ₄—N(R″″)—
—N(R″″)—C(O)—(CH₂)ₓ₄—N(R″″)—;
—(CH₂)ₓ₄—C(O)O— and
—(CH₂)ₓ₄—C(O)—
wherein
R″″ are independently of each other selected from H and C₁-C₄-alkyl
x2 represents an integer selected from 1, 2, 3 or 4; in particular 1 or 2;
x3 represents an integer selected from 1, 2, 3 or 4; in particular 1 or 2; and
x4 represents an integer selected from 1, 2, 3 or 4; in particular 1 or 2.
(Moiety M5); or
f) combinations of at least two identical or, more particularly, different moieties selected from M1, M2, M3, M4 and M5.

4. The compound of anyone of the preceding claims, wherein said linker group A is an enzymatically or chemically cleavable linker group selected from
a) a peptidyl group, in particular di-, tri- or tetra-peptidyl group;
b) a disulfide group of the formula —(CR⁷R⁸)ₙ₂—S—S—(CR⁷R⁸)ₙ₂—X₅— or X_{5′}—(CR⁷R⁸)ₙ₂—S—S—(CR⁷R⁸)ₙ₂—X₅—
wherein
n2 represents an integer from 1 to 4,
residues R⁷ and R⁸ independently of each other are selected from H or lower alkyl, in particular methyl; or two residues R⁷ and R⁸ together with the carbon atom which they are attached to form a cyclic C₄- to C₈-alkyl group; and
moiety X₅ is selected from —C(O)— and —O—;
moiety X_{5′} is selected from —C(O)— and —(O)C—(CH₂)—NH—;
c) hydrazone groups selected from >C═N—N(R⁹)— and —N(R⁹)—N═C<
wherein
R⁹ is H or lower alkyl; and
d) beta-glucuronidase-sensitive cleavable linker groups, in particular carrying a beta-glucuronic acid derived trigger residue

5. The compound of anyone of the preceding claims, wherein said self-immolative group X is selected from
a) p-amino-benzyl alcohol derived groups of the formula —NH-p-phenylene-CH₂—O— or —O—CH₂-p-phenylene-NH— or —NH-p-phenylene-CH₂—N⁺(R²⁰)₂—
b) —O—C(O)—O—;
c) —O—C(O)—NR¹⁰—(CR¹²R¹³)_{z}—NR¹¹—C(O)—O— or —X¹—C(O)—NR¹⁰—(CR¹²R¹³)_{z}—NR¹¹—C(O)—X²—
wherein
z represents an integer selected from 1 to 6, in particular 1 to 4;
R²⁰ independently of each other, represent H or a lower alkyl group R¹⁰ and R¹¹, independently of each other, represent H or lower alkyl group
R¹² and R¹³, independently of each other, represent H, methyl or ethyl, in particular H or methyl, especially H; and
X¹ and X² independently of each other represent O, S or NR¹⁰
d) methylene alkoxy carbamates (MAC) type linkages of the formula
—OC(O)—NR¹³—C(R¹⁴R¹⁵)—(O)—
—OC(O)—NR¹³—C(R¹⁴R¹⁵)—(S)—
—OC(O)—NR¹³—C(R¹⁴R¹⁵)—(NR¹⁶)— or
—OC(O)—NR¹³—C(R¹⁴R¹⁵)—(NR¹⁶—C(O)O)—
wherein
R¹³, R¹⁴, R¹⁵, and R¹⁶, independently of each other represent H or lower alkyl, in particular, C₁ to C₄-alkyl.

6. The compound of anyone of the preceding claims, wherein said payload residue Y is selected from bioactive compounds, labeling agents, such as in particular dyes, radiolabels and fluorophores, protein degraders, in particular payloads applicable in proteolysis targeting chimeras (PROTACs), photosensitizers, and chelators.

7. The compound of anyone of the preceding claims, wherein Sp¹ is selected from one of the following combinations of Moieties
-M1-M3-
-M2-M3-
-M3-M1-
-M3-M2-
wherein
the linkages between said moieties M1, M2, M3 are independently selected from a chemical bond, an ether, thioether, ester, amide, carbamate, dicarbamate, carbonate, hydrazine or urea, and alkylene oxide or a linear or branched polyalkylene oxide linkage;
and/or
wherein Sp² is selected from one of the following combinations of Moieties
-M1-M3-
-M1-M4-
-M2-M3-
-M2-M4-
-M2-M5-
-M3-M1-
-M3-M2-
-M3-M4-
-M1-M3-M4-
-M1-M4-M3-
-M2-M3-M4-
-M2-M4-M3-
-M3-M2-M4-
-M3-M4-M2-
-M2-M5-M4-
wherein
the linkages between said moieties M1, M2, M3, M4 and M5 are independently selected from a chemical bond, an ether, thioether, ester, amide, carbamate, dicarbamate, carbonate, hydrazine or urea, and alkylene oxide or a linear or branched polyalkylene oxide linkage.

8. A conjugate, obtainable by reacting a functionalized targeting agent, with a tetrazine compound of anyone of the preceding claims in order to form a covalent linkage between said functionalized targeting agent, and said tetrazine compound of formula I; in particular wherein said functionalized targeting agent is selected from viruses, whole cells, phages, liposomes, biomolecules and low-or-high-molecular weight chemical compounds, in particular antibodies, antibody derivatives, antibody fragments, antibody (fragment) fusions, enzymes, proteins, peptides, peptide mimetics, carbohydrates, monosaccharides, polysaccharides, oligo- or polynucleotides, in particular DNA, RNA, PNA and LNA molecules, aptamers, drugs, glycoproteins, glycans, lipids, polymers, chemotherapeutic agents, receptor agonists and antagonists, cytokines, hormones, steroids, toxins and derivatives thereof.

9. The conjugate of claim 8, wherein said functionalized targeting agent comprises as functional group at least one dienophilic moiety reactive with said tetrazine moiety of said compound of formula I.

10. The conjugate of anyone of the claims 8 or 9, wherein said functionalized targeting agent comprises at least one polypeptide sequence, having at least one non-natural amino acid residue within its amino acid sequence, which non-natural amino acid residue comprises at least one dienophile moiety reactive with said tetrazine moiety of said compound of formula I; in particular, wherein said functionalized biomolecule is a polyclonal or monoclonal immunoglobulin molecule, in particular a monoclonal antibody or fragment thereof.

11. The conjugate of anyone of the claims 8 to 10, which is formed by biorthogonal bioconjugation of a tetrazine-compound of formula I and a functionalized biomolecule carrying a functional group capable of reaction via a Diels-Alder-type cycloaddition reaction.

12. The conjugate of claim 11, wherein said functional group capable of reaction via a Diels-Alder-type cycloaddition reaction is selected from
(i) a trans-cyclooctenyl dienophile group of the formula: 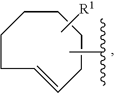
wherein
R¹ is hydrogen, halogen, C₁-C₄-alkyl, (R^{a}O)₂P(O)O—C₁-C₄-alkyl, (R^{b}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, Di-(C₁-C₄-alkyl)amino, C₂-C₅-alkenylamino, C₂-C₅-alkenyl-C₁-C₄-alkyl-amino or Di-(C₂-C₅-alkenyl)amino; and
R^{a}, R^{b} independently are hydrogen or C₂-C₅-alkanoyloxymethyl; or
(ii) a cyclooctynyl dienophile group of the formula: 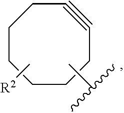
wherein
R² is hydrogen, halogen, C₁-C₄-alkyl, (R^{c}O)₂P(O)O—C₁-C₄-alkyl, (R^{d}O)₂P(O)—C₁-C₄-alkyl, CF₃, CN, hydroxyl, C₁-C₄-alkoxy, —O—CF₃, C₂-C₅-alkenoxy, C₂-C₅-alkanoyloxy, C₁-C₄-alkylaminocarbonyloxy or C₁-C₄-alkylthio, C₁-C₄-alkylamino, Di-(C₁-C₄-alkyl)amino, C₂-C₅-alkenylamino, C₂-C₅-alkenyl-C₁-C₄-alkyl-amino or Di-(C₂-C₅-alkenyl)amino; and
R^{c}, R^{d} independently are hydrogen or C₂-C₅-alkanoyloxymethyl.

13. A method of preparing a bio-conjugate of anyone of the claims 8 to 12, which method comprises reaction in an aqueous, optionally buffered reaction medium a tetrazine compound as defined in anyone of the claims 1 to 7 with a functionalized biomolecule carrying a functional dienophilic group and performing a Diels-Alder-type cycloaddition reaction between said molecules.

14. A tetrazine intermediate of the general formula II 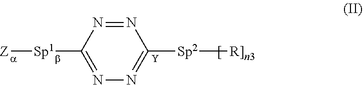
wherein
n3 represent an integer selected from 1 or 2;
Sp¹ and Sp² are as defined above,
linkages α, β, and γ are independently from each other selected from a chemical bond, or an ether, thioether, ester, amide, carbonyl, in particular keto, carbamate, dicarbamate, carbonate, hydrazine, urea, alkylene oxide or linear or branched polyalkylene oxide linkage;
Z represents a phosphor containing hydrophilic group, in particular (R¹O)₂P(O)—, (R^{1a}O)₂P(O)—O—, and (R²O)₃P—O—;
wherein
R¹, R^{1a} and R² are same or different and independently of each other represent H or lower alkyl, in particular methyl or ethyl; and even more particularly H;
and
R represents H or a chemical group capable of forming a chemical bond, or capable of forming an ether, thioether, ester, such as active esters like succinimidyl- or pentafluorophenyl-ester, amide, carbamate, dicarbamate, carbonate, hydrazine, urea, alkylene oxide or linear or branched polyalkylene oxide linkage; and more particularly R represents an amino or carboxyl group; and optionally with the proviso that R does not represent a chemical protecting group, in particular does not represents a cleavable protecting group, and more particularly not a N—, O—, or S-protecting group.

15. A method of preparing a tetrazine intermediate of general formula II which method comprising the steps of:
a) reacting (i) a first cyano compound of the general formula III
Z—Sp¹-CN (III)
wherein
Z and Sp¹ are as defined above, wherein optionally any hydroxyl group of residue Z is provided in protected, i.p. alkoxy, form;
with (ii) a second cyano compound of the general formula IV
NC—Sp²-[R]ₙ₃ (IV)
wherein
R and Sp² and n3 are as defined above;
in the presence of (iii) a hydrazine hydrate;
b) subsequent oxidation;
c) optionally isolating the obtained tetrazine compound;
and
d) optionally deprotecting the hydroxyl groups of residue Z.

16. A conjugate as defined in anyone of the claims 8 to 12, for use in medicine, in particular for use in diagnosis and/or therapy.

17. A pharmaceutical composition, comprising in a pharmaceutically acceptable carrier at least one conjugate as defined in anyone of the claims 8 to 12.

18. A diagnostic or analytical kit comprising at least one tetrazine compound as defined in anyone of the claims 1 to 7.
